SEQUENCE LISTING (18 sequences)

SEQ ID NO: 1 - DNA, 79 bp, synthetic construct
  source 1..79: mol_type = other DNA
gtgactggagttcagacgtgtgctcttccgatctggtagtcatcgtggtaaaaaaaaaaaaaaaaaaaaaaaaaaaaaa

SEQ ID NO: 2 - DNA, 79 bp, synthetic construct
  source 1..79: mol_type = other DNA
gtgactggagttcagacgtgtgctcttccgatctgtccgtattctgtcaaaaaaaaaaaaaaaaaaaaaaaaaaaaaaa

SEQ ID NO: 3 - DNA, 79 bp, synthetic construct
  source 1..79: mol_type = other DNA
gtgactggagttcagacgtgtgctcttccgatcttccctttatgcgaggaaaaaaaaaaaaaaaaaaaaaaaaaaaaaa

SEQ ID NO: 4 - DNA, 79 bp, synthetic construct
  source 1..79: mol_type = other DNA
gtgactggagttcagacgtgtgctcttccgatctaaagcatgaccagtaaaaaaaaaaaaaaaaaaaaaaaaaaaaaaa

SEQ ID NO: 5 - DNA, 79 bp, synthetic construct
  source 1..79: mol_type = other DNA
gtgactggagttcagacgtgtgctcttccgatcttacagtcgcttggcaaaaaaaaaaaaaaaaaaaaaaaaaaaaaaa

SEQ ID NO: 6 - DNA, 15 bp, synthetic construct
  source 1..15: mol_type = other DNA
gtcaactctttagcg

SEQ ID NO: 7 - DNA, 15 bp, synthetic construct
  source 1..15: mol_type = other DNA
tgatggcctattggg

SEQ ID NO: 8 - DNA, 15 bp, synthetic construct
  source 1..15: mol_type = other DNA
ttccgcctctctttg

SEQ ID NO: 9 - DNA, 15 bp, synthetic construct
  source 1..15: mol_type = other DNA
acccaccagtaagac

SEQ ID NO: 10 - DNA, 15 bp, synthetic construct
  source 1..15: mol_type = other DNA
ggtcgagagcattca

SEQ ID NO: 11 - DNA, 15 bp, synthetic construct
  source 1..15: mol_type = other DNA
aaagcattcttcacg

SEQ ID NO: 12 - DNA, 15 bp, synthetic construct
  source 1..15: mol_type = other DNA
cttgccgcatgtcat

SEQ ID NO: 13 - DNA, 15 bp, synthetic construct
  source 1..15: mol_type = other DNA
ctttgtctttgtgag

SEQ ID NO: 14 - DNA, 79 bp, synthetic construct
  source 1..79: mol_type = other DNA
gtgactggagttcagacgtgtgctcttccgatctacccacttcagttcgaaaaaaaaaaaaaaaaaaaaaaaaaaaaaa

SEQ ID NO: 15 - DNA, 79 bp, synthetic construct
  source 1..79: mol_type = other DNA
gtgactggagttcagacgtgtgctcttccgatctacgccggtagtatctaaaaaaaaaaaaaaaaaaaaaaaaaaaaaa

SEQ ID NO: 16 - DNA, 79 bp, synthetic construct
  source 1..79: mol_type = other DNA
gtgactggagttcagacgtgtgctcttccgatcttatcgtctccgactaaaaaaaaaaaaaaaaaaaaaaaaaaaaaaa

SEQ ID NO: 17 - DNA, 79 bp, synthetic construct
  source 1..79: mol_type = other DNA
gtgactggagttcagacgtgtgctcttccgatctaggtcctcctgtattaaaaaaaaaaaaaaaaaaaaaaaaaaaaaa

SEQ ID NO: 18 - DNA, 79 bp, synthetic construct
  source 1..79: mol_type = other DNA
gtgactggagttcagacgtgtgctcttccgatctatttcgccttgccagaaaaaaaaaaaaaaaaaaaaaaaaaaaaaa

(19) United States
(12) Patent Application Publication
(10) Pub. No.: US 2025/0093339 A1
(43) Pub. Date: Mar. 20, 2025

(54) COMPOSITIONS AND METHODS FOR ENHANCED SAMPLE MULTIPLEXING

(71) Applicant: BioLegend, Inc., San Diego, CA (US)

(72) Inventors: Andre VIEIRA ZORZETTO FERNANDES, San Diego, CA (US); Ze SHI, San Diego, CA (US); Zeinab MOUSSA, San Diego, CA (US); Fernando GRIGERA, San Diego, CA (US)

(21) Appl. No.: 18/718,336

(22) PCT Filed: Dec. 14, 2022
(86) PCT No.: PCT/US2022/081508

§ 371 Date: Jun. 10, 2024

Related U.S. Application Data
(60) Provisional application No. 63/426,457, filed on Nov. 18, 2022.
(60) Provisional application No. 63/290,060, filed on Dec. 15, 2021.

(51) Int. Cl. G01N 33/53; C12Q 1/6804; C12Q 1/6869

(52) U.S. Cl. CPC G01N 33/5308; C12Q 1/6804; C12Q 1/6869; G01N 2400/10; G01N 2458/10

(57) ABSTRACT

Provided herein are compositions, methods and systems that allow for detection of samples via compositions that bind ubiquitous ligands or targets, to improve the capability of multi-sample utilization in single cell sequencing.

### CROSS-REFERENCE

This application is a 35 U.S.C. 371 national phase application of International Patent Cooperation Treaty (PCT) Application No. PCT/US2022/081508, filed on Dec. 14, 2022, entitled COMPOSITIONS AND METHODS FOR ENHANCED SAMPLE MULTIPLEXING, naming Andre VIERA ZOEZETTO FERNANDES et al. as inventors, which claims the benefit of U.S. Provisional Application No. 63/290,060 filed on Dec. 15, 2021, and U.S. Provisional Application No. 63/426,457 filed on Nov. 18, 2022, each of which is incorporated in its entirety herein by reference.

### FIELD OF THE INVENTION

The present disclosure relates, in some aspects, to molecular biology methods. The disclosure further relates to identification of a sample thorough measurement of labeled cells. The disclosure further relates to simultaneous identification of a single cell and a sample of origin.

### BACKGROUND

The ability to characterize individual cells in a heterogeneous population is rapidly becoming a pivotal focus of biological research and clinical diagnostics, yet the massively parallel capability of single cell analysis in current technology has yet to maximize its potential to efficiently scale up the number of samples being processed per run. Current technologies aimed at increasing single cell sample size for analysis by including multiple cell samples have focused on antibody based reagents combined with oligonucleotides, which rely on expression and binding of specific antigenic epitopes. Although the combinatorial potential of oligonucleotides could theoretically allow for the identification of an almost unlimited number of samples, antibody based methods are still limited in their availability of antigen-specific antibodies. Moreover, the varied epitope expression between cell types, and changes in epitope expression under distinct culture conditions limit the potential of antibody based approaches. Therefore, there remains a need for compositions, methods and systems that allow for detection of samples via compositions that bind ubiquitous ligands or targets, to improve the capability of multi-sample utilization in single cell sequencing.

### BRIEF SUMMARY

Provided herein are compositions for sample identification, which include a first plurality of complexes each comprising a substantially identical glycan-binding agent conjugated to a universal anchor. In some embodiments, the universal anchor of the first plurality comprises an amine-modified oligonucleotide sequence. In some embodiments, the universal anchor of the first plurality comprises a locked nucleic acid (LNA).

In some embodiments, the universal anchor of the first plurality comprises at least 3 or more nucleotide bases which hybridize to a complementary sequence on a labeling oligonucleotide.

In some embodiments, the labeling oligonucleotide further comprises a binding site for a primer, a sample barcode sequence, and a capture sequence. In some embodiments, the binding site for the primer comprises the complementary sequence on the labeling oligonucleotide. In some embodiments, each sample barcode sequence within the first plurality comprises a substantially identical barcode sequence.

In some embodiments, one or more additional pluralities of complexes, wherein each complex within the one or more additional pluralities comprises a substantially identical glycan-binding agent conjugated to a universal anchor. In some embodiments, the glycan-binding agent from each of the one or more additional pluralities is substantially identical to a) the glycan-binding agent of the first plurality, and b) the glycan-binding agent of the one or more additional pluralities. In some embodiments, the glycan-binding agent from each of the one or more additional pluralities is substantially distinct from a) the glycan-binding agent of the first plurality, and b) any other glycan-binding agent of the one or more additional pluralities. In some embodiments, the universal anchor from the one or more additional pluralities comprises an amine-modified oligonucleotide sequence.

In some embodiments, the universal anchor from the one or more additional pluralities of complexes comprises a locked nucleic acid (LNA). In some embodiments, the universal anchor of the one or more additional pluralities comprises at least 3 or more nucleotide bases which hybridize to a complementary sequence on a labeling oligonucleotide. In some embodiments, the labeling oligonucleotide comprises a binding site for a primer, a sample barcode sequence, and a capture sequence. In some embodiments, the binding site for the primer comprises the complementary sequence on the labeling oligonucleotide. In some embodiments, the sample barcode sequence within each of the one or more additional pluralities comprises a substantially identical barcode sequence, and wherein the sample barcode sequence between the first plurality and the one or more additional pluralities comprises a distinct sample barcode sequence.

Provided herein are compositions for sample identification, comprising a first plurality of complexes, each comprising a glycan-binding agent conjugated to a universal anchor, and one or more additional pluralities of complexes each comprising a glycan-binding agent conjugated to a universal anchor. In some embodiments, the universal anchor is hybridized to a labeling oligonucleotide comprising a binding site for a primer, a sample barcode sequence and a capture sequence; and wherein the sample barcode sequence within each of the one or more additional pluralities comprises a substantially identical barcode sequence; and wherein the sample barcode sequence between the first plurality and the one or more additional pluralities comprises a distinct sample barcode sequence.

In some embodiments, the glycan-binding agent of the first plurality and the glycan binding agent of the one or more additional pluralities comprise a substantially identical glycan binding agent. In some embodiments, the glycan-binding agent of the first plurality and the glycan binding agent of the one or more additional pluralities comprise a distinct glycan binding agent. In some embodiments, the glycan-binding agent does not comprise an antibody. In some embodiments, the capture sequence hybridizes to a complementary sequence on a capture polymer. In some embodiments, the capture polymer is immobilized to a solid substrate. In some embodiments, the substrate is a bead, a microfluidics bead, a slide, a multi-well plate or a chip.

In some embodiments, the binding site for a primer comprises a polynucleotide sequence of at or about 10 nucleotide bases that provides an annealing site for amplification of the universal anchor. In some embodiments, the sample barcode sequence comprises a polynucleotide sequence of at least at or about 2 nucleotide bases. In some embodiments, the sample barcode comprises a spatial barcode sequence.

In some embodiments, the glycan-binding agent comprises a carbohydrate binding agent. In some embodiments, the glycan-binding agent comprises a glycan-binding protein (GBP).

In some embodiments, the glycan-binding agent comprises a lectin. In some embodiments, the glycan-binding agent is derived from a plant, an animal, a fungus, a microbe, or a parasite. In some embodiments, the glycan-binding agent is selected from concanavalin A (ConA), a wheat germ agglutinin (WGA), a pea lectin, a soybean lectin, a lentil lectin, a ricin, a bacterial toxin, an influenza virus hemagglutinin, a C-type lectin, an S-type lectin, a P-type lectin, or an I-type lectin. In some embodiments, the glycan-binding agent of the first plurality and the one or more additional pluralities comprises concanavalin A (ConA).

In some embodiments, the glycan-binding agent of the first plurality and the one or more additional pluralities comprises wheat germ agglutinin (WGA). In some embodiments, the glycan-binding agent of the first plurality comprises concanavalin A (ConA) and the glycan-binding agent of the one or more additional pluralities comprises wheat germ agglutinin (WGA). In some embodiments, any of the compositions provided herein further contains a detectable label. In some embodiments, the detectable label is a tag or a fluorescent label.

In some embodiments, the glycan-binding agent binds to all or a portion of a ligand on the one or more target cells. In some embodiments, the ligand comprises all or a portion of a glycan on one or more target cells. In some embodiments, the ligand comprises a common core structure of a glycan. In some embodiments, the common core structure is a pentacaccharide. In some embodiments, the pentasaccharide is Man₃GlcNAc₂. In some embodiments, the common core structure comprises an N-linked glycan. In some embodiments, the common core structure comprises an O-linked glycan.

In some embodiments, the one or more target cells comprise a first cell type and a second cell type. In some embodiments, the first cell type and the second cell type comprise human cells.

In some embodiments, the first cell type and the second cell type comprise non-human cells.

In some embodiments, the first cell type comprises human cells and the second cell type comprises non-human cells. In some embodiments, the one or more target cells comprise an immune cell, a non-immune cell, a hematopoietic cell, a blood cell, a stem cell, an epithelial cell, an endothelial cell, a nerve cell, a muscle cell, a fat cell, a bone cell, a reproductive cell, a lung cell, a cardiac cell, a tumor cell or a cancer cell. In some embodiments, the one or more target cells comprise cancer cells.

In some embodiments, the one or more target cells comprise immune cells. In some embodiments, the immune cells comprise lymphocytes or a myeloid cells. In some embodiments, the immune cells comprise T cells, B cells, monocytes, basophils, eosinophils, neutrophils, or natural killer cells. In some embodiments, the glycan is on the surface of a target cell. In some embodiments, the glycan is on a membrane of a target cell. In some embodiments, the glycan is in the intracellular space of a target cell. In some embodiments, the glycan comprises a glycoconjugate, a glycoprotein, glycolipid, a proteoglycan, a galectin or a SIGLEC. In some embodiments, the glycan comprises a differentiation marker.

In some embodiments, glycan comprises an antigenic determinant. In some embodiments, the glycan binding agent of the first plurality binds to all or a portion of a target on a first cell type, and the glycan binding agent of each of the one or more additional pluralities binds to all or a portion of a target on a second cell type. In some embodiments, the first cell type and the second cell type comprise human cells. In some embodiments, the first cell type and the second cell type comprise non-human cells. In some embodiments, the first cell type comprises human cells and the second cell type comprises non-human cells. In some embodiments, the first plurality comprises 2 or more glycan-binding agents conjugated to a universal anchor.

In some embodiments, the first plurality comprises between 1 to 100 cell glycan-binding agents conjugated to a universal anchor. In some embodiments, the first plurality comprises 100 or more glycan-binding agents conjugated to a universal anchor. In some embodiments, the first plurality of comprises 1000 or more glycan-binding agents conjugated to a universal anchor. In some embodiments, each of the one or more additional pluralities comprises 2 or more glycan-binding agents conjugated to a universal anchor. In some embodiments, each of the one or more additional pluralities comprises between 1 to 100 glycan-binding agents conjugated to a universal anchor. In some embodiments, each of the one or more additional pluralities comprises 100 or more glycan-binding agents conjugated to a universal anchor.

In some embodiments, each of the one or more additional pluralities comprises 1000 or more glycan-binding agents conjugated to a universal anchor. In some embodiments, the one or more additional pluralities of comprises 1 plurality of complexes. In some embodiments, the one or more additional pluralities comprises between 1 to 100 pluralities of complexes.

In some embodiments, the one or more additional pluralities comprises 10 or more pluralities of complexes. In some embodiments, the one or more additional pluralities comprises 20 or more pluralities of complexes. In some embodiments, the one or more additional pluralities comprises 30 or more pluralities of complexes. In some embodiments, the one or more additional pluralities comprises 40 or more pluralities of complexes. In some embodiments, the one or more additional pluralities comprises 50 or more pluralities of complexes.

In some embodiments, the one or more additional pluralities comprises 100 or more pluralities of complexes. In some embodiments, the one or more additional pluralities comprises 1000 or more pluralities of complexes.

Provided herein is a kit containing any of the compositions described herein. In some embodiments, the first plurality and the one or more additional pluralities are packaged separately.

Provided herein is a kit comprising a) a plurality of substantially identical complexes comprising a glycan-binding agent and a universal anchor; b) a first plurality of labeling oligonucleotides comprising a binding site for a primer, a substantially identical sample barcode sequence and a capture sequence; and c) one or more additional pluralities of labeling oligonucleotides, wherein each labeling oligonucleotide comprises a binding site for a primer, a substantially identical sample barcode sequence and a capture sequence; wherein the sample barcode sequence between each of the first and one or more additional pluralities of labeling oligonucleotides comprises a distinct sample barcode sequence from the first plurality of labeling oligonucleotides or any other additional pluralities of labeling oligonucleotides.

Provided herein is a kit comprising, a) a first plurality of complexes comprising substantially identical glycan-binding agents conjugated to a universal anchor; b) one or more additional pluralities of complexes, each plurality comprising a substantially identical glycan-binding agent conjugated to a universal anchor; wherein the glycan binding agent between the first plurality and the one or more additional pluralities comprises a distinct glycan binding agent; c) a first plurality of labeling oligonucleotides comprising a binding site for a primer, a substantially identical sample barcode sequence and a capture sequence; and d) one or more additional pluralities of labeling oligonucleotides comprising a binding site for a primer, a substantially identical sample barcode sequence and a capture sequence; wherein the sample barcode sequence between each of the first and one or more additional pluralities of labeling oligonucleotides comprises a distinct sample barcode sequence. In some embodiments, each component is packaged separately.

Provided herein is a method of generating a pool of samples for sequencing, comprising generating a first sample by contacting the first plurality of complexes of any of the embodiments described herein with a first plurality of target cells comprising one or more glycans, wherein the first plurality of complexes bind to the one or more glycans; generating one or more additional samples by contacting the one or more additional pluralities of complexes of any of the embodiments described herein with one or more additional pluralities of target cells each comprising one or more glycans, wherein the one or more additional pluralities of complexes bind to the one or more glycans, and pooling the first and the one or more additional samples.

Provided herein is a method of identifying a sample, comprising generating a first plurality of samples by contacting the first plurality of complexes of any of the embodiments described herein with a first plurality of target cells comprising one or more glycans, wherein the first plurality of complexes bind to the one or more glycans, generating one or more additional pluralities of samples by contacting the one or more additional pluralities of complexes of any of the embodiments described herein with one or more additional pluralities of target cells each comprising one or more glycans, wherein the one or more additional pluralities of complexes bind to the one or more glycans, pooling the first and the one or more additional pluralities of samples, partitioning each sample from the pool of first and one or more additional pluralities of samples into a plurality of partitions, wherein each partition of the plurality of partitions comprises a single cell associated with a complex, within the partition, hybridizing the capture sequence on the complex associated with a capture polymer on a solid substrate, obtaining sequence information from the pool of first and one or more additional pluralities of samples to identify the first and one or more additional pluralities of target cells from the pool generated as described above.

Provided herein is a method of identifying a first cell type and one or more additional cell types from a mixture of cells, comprising generating a first plurality of samples by contacting the first plurality of complexes of any of the embodiments described herein with a first plurality of target cells comprising one or more glycans, wherein the first plurality of complexes bind to the one or more glycans, generating one or more additional pluralities of samples by contacting the one or more additional pluralities of complexes of any of the embodiments described herein with one or more additional pluralities of target cells each comprising one or more glycans, wherein the one or more additional pluralities of complexes bind to the one or more glycans, pooling the first and the one or more additional pluralities of samples, partitioning each sample from the pool of first and one or more additional pluralities of samples into a plurality of partitions, wherein each partition of the plurality of partitions comprises a single cell associated with a complex, within the partition, hybridizing the capture sequence on the complex with a capture polymer on a solid substrate obtaining sequence information from the pool of first and one or more additional pluralities of samples to identify the first and one or more additional pluralities of target cells from the pool generated as described above.

In some embodiments, the first plurality of target cells and the one or more additional pluralities of target cells are selected from the group comprising human cells, non-human cells, healthy cells, cells associated with a disease or disorder, cancer cells, non-cancer cells, cells from the same subject, cells from a different subject, activated cells, non-activated cells, fresh cells, fixed cells, permeabilized cells, cells from a human subject or cells from a cell line.

In some embodiments, the cancer is selected from leukemia, polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia, myelodysplasia, sarcoma, carcinoma, fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancer, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyngioma, ependymoma, pinealoma, hemangioblastoma, acoustic neuroma, oligodendroglioma, meningioma, melanoma, neuroblastoma and retinoblastoma). In some embodiments, any of the methods described herein further includes lysing the cell after hybridizing the capture sequence to the capture polymer.

Provided herein is a method of identifying a sample through sequential hybridization, comprising contacting a first plurality of complexes each comprising a substantially identical glycan-binding agent conjugated to a universal anchor with a first plurality of target cells comprising one or more glycans, wherein the first plurality of complexes bind to the one or more glycans, reversibly hybridizing the first plurality of complexes to a complementary sequence on a first labeling oligonucleotide comprising a binding site for a primer, a sample barcode sequence, a capture sequence, and a detectable label, detecting the detectable label, de-hybridizing the first plurality of complexes, reversibly hybridizing the first plurality of complexes to a complementary sequence on a second labeling oligonucleotide comprising a binding site for a primer, a sample barcode sequence, a capture sequence, and a detectable label, detecting the detectable label identifying the sample based on the detectable labels.

Provided herein is a method of identifying a sample through sequential hybridization, comprising contacting a first plurality of complexes each comprising a substantially identical glycan-binding agent conjugated to a universal anchor with a first plurality of target cells comprising one or more glycans, wherein the first plurality of complexes bind to the one or more glycans, contacting one or more additional pluralities of complexes, each plurality comprising a distinct glycan-binding agent conjugated to a universal anchor, with the first plurality of target cells comprising one or more glycans, wherein the one or more additional pluralities of complexes bind to the one or more glycans, incubating the first plurality of complexes and the one or more additional pluralities of complexes with a first labeling oligonucleotide comprising a binding site for a primer, a sample barcode sequence, a capture sequence, and a detectable label, under conditions to reversibly hybridize the first labeling oligonucleotide to the universal anchor, detecting the detectable label, de-hybridizing the first plurality of complexes and the one or more additional pluralities of complexes, incubating the first plurality of complexes and the one or more additional pluralities of complexes with a second labeling oligonucleotide comprising a binding site for a primer, a sample barcode sequence, a capture sequence, and a detectable label, under conditions to reversibly hybridize the second labeling oligonucleotide to the universal anchor, detecting the detectable label, and identifying the sample based on the detectable labels.

In some embodiments, the sample barcode sequence on the second labeling oligonucleotide is different than the sample barcode sequence from the first labeling oligonucleotide. In some embodiments, the detectable label on the second labeling oligonucleotide is different than the detectable label from the first labeling oligonucleotide.

Provided herein is a method of associating presence or abundance of a glycan with a location of a tissue sample, comprising delivering the first plurality of complexes of any of the embodiments described herein to a tissue sample affixed to a support, under conditions to specifically bind the first plurality of complexes to the glycan at a location of the tissue sample, and sequencing all or a portion of the first plurality of complexes, and using the determined sequence to associate presence or abundance of the target biological molecule with the location of the tissue sample.

In some embodiments, the method further includes delivering the one or more additional pluralities of complexes of any of the embodiments described herein to a tissue sample affixed to a support, under conditions to specifically bind the one or more additional pluralities of complexes to the glycan at a location of the tissue sample, and sequencing all or a portion of the one or more additional pluralities of complexes, and using the determined sequence to associate presence or abundance of the target biological molecule with the location of the tissue sample.

In some embodiments, the sample comprises a sub-cellular component. In some embodiments, the sub-cellular component is isolated. In some embodiments, the sub-cellular component is selected from the group consisting of a nucleus, ribosome, an endoplasmic reticulum, a Golgi apparatus, a cytoskeleton, a mitochondria, a vacuole and a vesicle. In some embodiments, the sub-cellular component is a nucleus.

### BRIEF DESCRIPTION OF THE DRAWINGS

FIGS. 1A-1D depict uniform manifold approximation and projection (UMAP) plots of fresh and fixed cell populations of mouse bone marrow (mBM), mouse thymocytes (mThy), hPBMC_PHA, hPBMC_CAC, hPBMC_rest, labeled with exemplary HTOs, exemplary lectin-biotin conjugates and an oligonucleotide conjugated anti-biotin antibody. Fresh cells were demultiplexed by HTOs (FIG. 1A), fixed cells demultiplexed by with HTOs (FIG. 1B), fresh cells demultiplexed by exemplary lectin-biotin conjugates (FIG. 1C) and fixed demultiplexed by exemplary lectin-biotin conjugates (FIG. 1D).

FIGS. 2A and 2B depict uniform manifold approximation and projection (UMAP) plots of cell populations of B16-F0, Ba/F3, C2C12, 1929 and NIH/3T3 cells labeled with HTOs (FIG. 2A), or with exemplary lectin-biotin conjugates (FIG. 2B).

FIGS. 3A-3F show uniform manifold approximation and projection (UMAP) plots of cell populations either CAC stimulated or non-stimulated and treated or not treated with exemplary lectins. Cells were demultiplexed as doublets (FIG. 3A), negative (FIG. 3D), unstim_with_lectin (FIG. 3B), unstim_no_lectin (FIG. 3E), CAC_6 h_with_lectin (FIG. 3C) and CAC_6 h_no_lectin (FIG. 3F).

FIGS. 4A and 4B depict staining of isolated nuclei in the absence of biotinylated ConA (−ConA; FIG. 4A) or the presence of biotinylated ConA (+ConA; FIG. 4B). Biotin detection was performed using anti-biotin PE (BioLegend cat #409003), and identification was performed using flow cytometric analysis.

FIGS. 5A-5H depict histograms of different cell populations stained with exemplary tested lectins in human and murine cells. FIG. 5A and FIG. 5B depict human and murine cells stained with fucose dependent lectins, respectively. FIG. 5C depicts human cells stained with mannose dependent lectins. FIG. 5D and FIG. 5E depict human and murine cell populations stained with GlcNAc dependent lectins, respectively. FIG. 5F depicts human cell populations stained with sialic acid containing glycans. FIG. 5G depicts human cell populations stained with galactose binding lectins. FIG. 5H depicts human cell populations stained with GalNAc binding lectins.

FIGS. 6A-6C depict western blots demonstrating the effect of various conjugation methods on lectins and antibodies. FIG. 6A depicts conditions for glycan conjugation on both antibodies and lectins. FIG. 6B depicts conditions to conjugate an oligonucleotide to periodate-treated IgG. FIG. 6C depicts conditions to conjugate a plant lectin to periodate-treated IgG.

FIGS. 7A and 7B depict results for exemplary lectin-direct conjugates purified under three conditions, SEC only, AEX only, or AEX and SEC. FIG. 7A depicts an results of an agarose gel with staining of DNA for samples purified under each condition. FIG. 7B depicts results of exemplary lectin-direct conjugates hybridized with a fluorescent label, and analyzed by flow cytometry.

FIGS. 8A and 8B depict uniform manifold approximation and projection (UMAP) plots of fresh human PBMC's labeled with HTOs (FIG. 8A) or lectin-direct conjugates (FIG. 8B).

FIGS. 9A-9G depict immunohistochemistry images of FFPE samples from a first donor (D1) or second donor (D2) co-stained with antibodies and lectins. FIG. 9A depicts melanoma cells and non-cancerous cells (NAT) stained with Melan-A, Jacalin and DAPI. FIG. 9B, depicts melanoma cells and non-cancerous cells (NAT) stained with Melan-A, LEL, TL and DAPI. FIG. 9C, depicts lymph node cells stained with Melan-A, Jacalin, LEL, TL and DAPI. FIG. 9D, depicts melanoma cells and non-cancerous cells (NAT) stained with nuclear Sox10, Jacalin and DAPI. FIG. 9E, depicts colorectal carcinoma cells and non-cancerous cells (NAT) stained with EpCAM, SNL, EBL, and DAPI. FIG. 9F, depicts colorectal carcinoma cells and non-cancerous cells (NAT) stained with EpCAM, UEA-I, and DAPI. FIG. 9G, depicts melanoma cells and non-cancerous cells (NAT) stained with nuclear Sox10, SNL, EBL and DAPI.

### DETAILED DESCRIPTION

Provided herein are compositions for identifying a sample from a heterogenous mixture of cells. Also provided herein are uses and methods of use thereof, articles of manufacture and kits comprising such compositions. In some embodiments, the compositions provided herein can be used for augmenting sample multiplexing for single cell analysis. In some embodiments, a composition may have a first, and optionally one or more additional pluralities of complexes each comprising a glycan-binding agent conjugated to a universal anchor. In some embodiments, the universal anchor is reversibly hybridized to complementary sequence on a labeling oligonucleotide. In some embodiments, the first plurality of complexes may have the same sample barcode sequence. In some embodiments, each plurality of the one or more additional pluralities of complexes may each may have the same sample barcode sequence. In some embodiments, each complex the first plurality of complexes may have a different sample barcode sequence. In some embodiments, the first and one or more additional pluralities of complexes between each plurality of complexes may each have a different sample barcode sequence. In some embodiments, each of the first and/or one or more additional pluralities of complexes may have the same or substantially the same glycan binding agent. In some embodiments, each of the first and/or one or more additional pluralities of complexes may have a different or distinct glycan binding agent. In some embodiments, the glycan binding agent binds to all or a portion of a ligand on a target cell. In some embodiments, the glycan binding agent binds to all or a portion of a glycan on a target cell. In some embodiments, the glycan binding agent binds to a common core structure of a glycan on a target cell.

In some embodiments, provided herein are uses and methods of use thereof, and articles of manufacture and kits including such compositions, for use in identifying a sample of origin from a heterogenous cell population and/or a mixture of samples. In some embodiments, the compositions provided herein can be used for identifying a sample of origin from a mixture of cells. In some embodiments, the mixture of cells includes different samples. In some embodiments, the mixture of cells includes aliquots from the same sample. In some embodiments, the mixture of cells includes human samples. In some embodiments, the mixture of cells includes human samples. In some embodiments, the mixture of cells includes non-human samples. In some embodiments, the mixture of cells includes human and non-human samples. In some embodiments, the mixture of cells includes cancer and non-cancer cells. In some embodiments, the mixture of cells includes cancer cells. In some embodiments, the mixture of cells includes activated cells. In some embodiments, the mixture of cells includes non-activated cells. In some embodiments, the mixture of cells includes activated and non-activated cells. In some of any embodiments, the compositions provided herein can be used alone or in combination with single cell analysis. In some of any embodiments, the compositions provided herein can be used to identify a sample of origin from a mixture of cells from different samples being analyzed using high throughput single cell detection methods.

In some aspects, the provided compositions offer advantages compared to conventional agents for identifying a sample of origin from a mixture of cells. In certain available methods designed to identify a sample of origin, detection is carried out using agents that recognize extracellular protein markers and/or single-cell transcriptomes in a high-throughput fashion, converting protein detection and/or RNA detection into a quantitative readout (US20180251825, Krutzik et al. Nat Meth. 2006; Lai et al. Cytometry 87, 2015). Although technologies such as SCITO-seq (Hwang et al. Nature Methods, 2021) have enabled highly scalable profiling of cells through the use of single cell combinatorial indexing, they are generally limited by the availability of cell surface markers, especially during disease and/or cellular activation states. Such available reagents may not be entirely satisfactory to properly identify distinct samples from a mixture of cells from either non-diseased or diseased populations.

Glycans are oligosaccharide/carbohydrate-based polymers present on all living organisms and can be found on a highly diverse set of structures, including proteins (glycoproteins), lipids (glycolipids) and polymers (glycopolymers). The majority of glycan structures are found on the cell surface, but can also be detected intracellularly in the cytoplasm and nucleus. In contrast to nucleic acids and antibodies, glycans are more resistant against denaturation, and due to their small size, contain higher densities of per unit surface area, allowing for multivalent interactions, which enhance binding affinity (Houseman et al. Chem Biol. 2002). Glycan binding-agents, including glycan-binding proteins (GBPs) are capable of binding defined carbohydrate epitopes and glycosidic linkages, and GBP-glycan binding pairs are capable of providing broad-spectrum interactions. A specific class of GBPs, termed lectins, exhibit relatively high affinity and specificity for oligosaccharides of glycoproteins and glycolipids. Certain lectins are capable of binding to a common core structure of a glycan, irrespective of additional glycosylation. Although current fluorescence based lectin array assays have enabled multiple binding interactions to be observed simultaneously (Hu et al. Proteomics Clin Appl; 2010), and recent updates to glycan based profiling methods have allowed for their application into single-cell sequencing methods (Minoshima et al. iScience, 2021), there is currently no glycan-based technology that allows for enhancing sample multiplexing for either single cell or spatial analysis.

Thus, the provided compositions in some embodiments overcome one or more of these challenges, including challenges related to cellular heterogeneity within or between distinct cellular samples. The provided compositions include pluralities of complexes capable of binding multiple cell surface molecules simultaneously through recognition of a common core structure. In some embodiments, the compositions provided herein are capable of indiscriminate binding of multiple distinct cell types. In some embodiments, the provided compositions are capable of increasing single cell library sequencing counts without the need to rely on PCR amplification. In some embodiments, the provided compositions are capable of reducing the signal to noise ratios and/or background noise associated with single cell sequencing methods.

All references cited herein, including patent applications, patent publications, and scientific literature and databases, are herein incorporated by reference in their entirety for all purposes to the same extent as if each individual reference were specifically and individually indicated to be incorporated by reference.

For clarity of disclosure, and not by way of limitation, the detailed description is divided into the subsections that follow. The section headings used herein are for organizational purposes only and are not to be construed as limiting the subject matter described.

### I. Glycans and Glycan-Binding Agents

Provided herein in some aspects are glycan-binding agents that bind to or recognize all or a portion of a ligand on a target cell. In some embodiments, the ligand on the target cell is all or a portion of a glycan. In some embodiments, the ligand on the target cell is a common core structure of a glycan. In some embodiments, the provided glycan-binding agents bind to or recognize more than one ligand and/or glycan on a target cell. In some embodiments, the provided glycan-binding agents bind to or recognize the same ligand and/or glycan on multiple target cells. In some embodiments, the provided glycan-binding agents bind to or recognize a different ligand and/or glycan on multiple target cells. In some embodiments, the provided glycan-binding agents bind to or recognize a common core structure of a glycan on a target cell. In some embodiments, the provided glycan-binding agents bind to or recognize a common core structure of a glycan on multiple target cells. In some embodiments, the ligand and/or glycan can be any of the glycans described herein. In some embodiments, the glycan-binding agent can be any of the glycan-binding agents described herein.

a. Glycans and Ligands

In some embodiments, the ligand described herein comprises all or a portion of a glycan. In some embodiments, the glycan is present on the surface of a target cell. In some embodiments, the glycan is present in the intracellular (IC) space of a target cell. In some embodiments, the glycan is on the membrane of a target cell. In some embodiments, the glycan on the target cell comprises a glycoconjugate, a glycoprotein, glycolipid, a proteoglycan, a galectin or a SIGLEC. In some embodiments, the glycan is a glycoconjugate, a glycoprotein, glycolipid, a proteoglycan, a galectin or a SIGLEC that is covalently bound to a carbohydrate.

In some embodiments, the glycan comprises a core structure. In some embodiments, the core structure is expanded through galactosylation, further GlcNAclyation, sialylation or fucosylation. In some embodiments, the glycan is the result of glycosylation of a protein or a lipid. In some embodiments, the glycan is an N-linked glycan, and O-linked glycan or a GPI-linked glycan. In some embodiments, the glycan is an N-linked glycan. In some embodiments, the glycan is an O-linked glycan. In some embodiments, the glycan is a GPI-linked glycan. In some embodiments, the N-linked glycan contains a common core pentasaccharide. In some embodiments, the common core pentasaccharide is Man3GlcNAc2. In some embodiments, the common core is fucosylated. In some embodiments, the O-linked glycan core is defined on the basis of the glycans attached to the initial N-acetylgalactosamine (GalNAc); for example, core 1 is GalNAc with β1,3-linked galactose.

In some embodiments, the glycan has at least at or about 5 residues. In some embodiments, the glycan has at least 5 residues. In some embodiments, the glycan has 5 residues.

In some embodiments, the glycan is a homopolymer. In some embodiments, the glycan is a heteropolymer. In some embodiments, the glycan is a linear glycan. In some embodiments, the glycan is branched glycan. In some embodiments, the glycan contains a structure used as a marker of cell differentiation (differentiation marker). In some embodiments, the differentiation marker is used to identify an activated cell from a non-activated cell. In some embodiments, the glycan contains a structure used as a marker of antigenic determination (antigenic determinant).

In some embodiments, any of the glycans described herein is a sugar, a monosaccharide, an oligosaccharide, a polysaccharide, a disaccharide, a polyol, a malto-oligosaccharide, a non-malto-oligosaccharide, a starch, a non-starch polysaccharide, a mucin, a collagen, a transferrin, a ceruloplasmin, a calnexin, a calreticulin, a glucose, a galactose, a fructose, a xylose, a sucrose, a lactose, a maltose, a trehalose, a sorbitol, a mannitol, a maltodextrin, a raffinose, a stachyose, a fructo-oligosaccharid, an amylose, an amylopectin, a modified starch, a glycogen, a cellulose, a hemicellulose, a pectin, a hydrocolloid, a α-D-mannosyl residue, a α-D-glucosyl residue, a branched α-mannosidic structure of high α-mannose type, a branched α-mannosidic structure of hybrid type and biantennary complex type N-Glycan, a fucosylated core region of bi- and triantennary complex type N-Glycan, a α1-3 and a 1-6 linked high mannose structure, Galβ1-4GalNAcβ1-R, Galβ1-3GalNAcα1-Ser/Thr, (Sia)Galβ1-3GalNAcα1-Ser/Thr, GalNAcα-Ser/Thr, GlcNAcβ1-4GlcNAcβ1-4GlcNAc, Neu5Ac (sialic acid), Neu5Acα2-6Gal(NAc)-R, Neu5Ac/Gca2,3Galβ1,4Glc(NAc), Neu5Ac/Gca2,3Galβ1,3(Neu5Acα2,6)GalNac, Fucα1-2Gal-R, Fucα1-2Galβ1-4(Fucα1-3/4)Galβ1-4GlcNAc, R2-GlcNAcβ1-4(Fucα1-6)GlcNAc-RI, or any derivative or combination thereof.

b. Glycan-Binding Agent

In some embodiments, any of the glycan-binding agents described herein is a carbohydrate binding agent. In some embodiments, any of the glycan-binding agents provided herein is a glycan-binding protein (GBP). In some embodiments, the GBP is a lectin. In some embodiments, the GBP is a glycosaminoglycan-binding protein. In some embodiments, the glycan-binding agent binds all or a portion of a ligand on a target cell. In some embodiments, the glycan-binding agent binds all or a portion of a ligand on one or more target cells. In some embodiments, the glycan-binding agent binds all or a portion of a glycan on a target cell. In some embodiments, the glycan-binding agent binds all or a portion of a glycan on one or more target cells. In some embodiments, the glycan-binding agent is not an antibody.

In some embodiments, the glycan-binding agent binds to or recognizes the common core structure of a glycan. In some embodiments, the glycan-binding agent binds to the common core structure of a glycan regardless of the glycosylation or expansion of the structure.

In some of any embodiments the glycan-binding agent is derived from a plant, an animal, a fungus, a microbe, or a parasite.

In some embodiments, the glycan-binding agent is a concanavalin A (ConA), a wheat germ agglutinin (WGA), a pea lectin, a soybean lectin, a lentil lectin, a ricin, a bacterial toxin, an influenza virus hemagglutinin, a C-type lectin, an S-type lectin, a P-type lectin, an I-type lectin, a Phaseolus vulgaris (red kidney bean) erythroagglutinin, a Lens culinaris (lentil) agglutinin, a Datura stramonium agglutinin, a Sambucus nigra (elderberry bark) agglutinin, a Snowdrop lectin (GNA), a Peanut agglutinin (PNA), a Jacalin (AIL), a Hairy vetch lectin (VVL), a Maackia amurensis leukoagglutinin (MAL), a Maackia amurensis hemoagglutinin (MAH), an Ulex europaeus agglutinin (UEA), or an Aleuria aurantia lectin (AAL).

In some embodiments, the glycan-binding agent interacts with Mannose and Glucose. In some embodiments, the glycan-binding agent is a ConA and interacts with Mannose and Glucose. In some embodiments, the glycan-binding agent interacts with Glucose and N-acetylneuraminic acid (NeuAc). In some embodiments, the glycan-binding agent is a WGA and interacts with Glucose and N-acetylneuraminic acid (NeuAc). In some embodiments, the interaction between the glycan and the glycan-binding agent is stabilized by hydrogen bonding between amino acids in the carbohydrate-recognition domain (CRD) and the glycan hydroxyl groups, and by van der Waals packing of the hydrophobic glycan face against the aromatic amino acid side chain (Weis and Drickamer, 1996).

Lectin AAL recognizes αFuc as predominant binding motif and it can tolerate this motif in many contexts (al-2, 1-3, 1-4, and 1-6). However, this lectin is inhibited by terminal GalNAc al-3 and Gal al-3Gal, associated with blood groups A and B respectively. By contrast, lectins LTL and UEA-I share affinity for the Lewis^{Y} antigen, although possibly by different interactions. While LTL favors glyco-epitopes with Fuc al-3 present in both Le^{X} and Le^{Y}, UEA-I binds motifs with Fuc al-2 identified only in Le^{Y}. Additionally, UEA-I better tolerates sulfating at C6 on the galactose molecule, a glyco-epitope frequent in secreted and matrix proteins. Lectins LCA and PSA preferentially bind to Fuc al-6 commonly (but not exclusively) associated to the core of N-glycans. Both proteins present a similar behavior toward tolerating different degrees of branching and terminal groups, and inhibition due to different Fuca linkages. Additionally, LCA, but not PSA, can also interact with terminal Man al-2, present in hybrid N-glycans.

Mannose lectins share affinity toward the highly abundant trimannoside core common to all N-glycans, however they differ from each other on the tolerance for modifications. NPL prefers polymannose structures containing α-1,6 linkages that are identified in high-mannose and hybrid glycans. Although it can also bind α-1,3 linked mannose, it is suggested that this interaction has lower affinity and strong interaction can only be achieved by the simultaneous binding of multiple sites.

Although often annotated as GlcNAC binding, due to its principal recognition of glycans with this termination, WGA shows enhanced binding when this epitope is present on long polyLacNAc chains, multi-antennary N-glycans, or longer chitin oligomers. Additionally, it can also recognize a plethora of N-acetyl-containing glycans including GlcNAc α-, GalNAc α-, GalNAc β-, MurNAc β-, and sometimes Neu5Ac. GSL-II also binds terminal GlcNAc β-, but it shows preference for GlcNAc-capped LacNAc in multiantennary N-glycans, or on polyLacNAc. Also, unlike WGA, GSL-II has been reported to favor tri- or tetra-antennary agalactosylated residues, which are rare on N-glycans, and fail to recognize glycans modified with terminal sialic acids. Lectins LEL and STL, share a relatively common affinity profile but while LEL favors glycans containing polyLacNac, STL prefers glycans with chitin polymers.

Both SBA and VVL have similar preferences and binding affinities, and reportedly prefer terminal GalNAcβ in the form of LacdiNAc and are inhibited by 3′-substitution on the proximal residue. Also, they bind terminal GalNAcα with specific limitations, SBA is inhibited by adjacent fucosylation, and VVL prefers simple sugar epitopes (1 or 2). Targets and Target Cells

In some aspects, the target is a target cell. In some embodiments, the target cell of any of the embodiments described herein is a human cell. In some aspects, the target cell of any of the embodiments described herein is a non-human cell. In some embodiments, one or more targets cells are human cells. In some embodiments, one or more target cells are non-human cells. In some embodiments, one or more targets cells include both human cells and non-human cells. In some embodiments, the target cell is a first cell type. In some embodiments, the target cell is a second cell type. In some embodiments, the target cell is or contains one or more target cells.

In some embodiments, the first cell type and the second cell type are selected from the group comprising human cells, non-human cells, healthy cells, cells associated with a disease or disorder, cancer cells, non-cancer cells, cells from the same subject, cells from a different subject, activated cells, non-activated cells, fresh cells, fixed cells, permeabilized cells, cells from a human subject or cells from a cell line. In some embodiments, the first cell type and the second cell type comprise human cells. In some embodiments, the first cell type and the second cell type comprise non-human cells. In some embodiments, the first cell type comprises human cells and the second cell type comprises non-human cells.

In some embodiments, the target cell is selected from an immune cell, a non-immune cell, a hematopoietic cell, a blood cell, a stem cell, an epithelial cell, an endothelial cell, a nerve cell, a muscle cell, a fat cell, a bone cell, a reproductive cell, a lung cell, a cardiac cell, an organ cell, a tumor cell or a cancer cell. In some embodiments, one or more target cells include an immune cell, a non-immune cell, a hematopoietic cell, a blood cell, a stem cell, an epithelial cell, an endothelial cell, a nerve cell, a muscle cell, a fat cell, a bone cell, a reproductive cell, a lung cell, a cardiac cell, an organ cell, a tumor cell or a cancer cell.

In some embodiments, the target cell is an immune cell. In some embodiments, the one or more target cells are immune cells. In some embodiments, the immune cell is a lymphocyte or a myeloid cell. In some embodiments, the one or more immune cells are a lymphocyte and a myeloid cell. In some embodiments, the immune cell is selected from the group consisting of T cells, B cells, monocytes, basophils, eosinophils, neutrophils, or natural killer cells. In some embodiments, the one or more target cells include T cells, B cells, monocytes, basophils, eosinophils, neutrophils, or natural killer cells.

In some of any embodiments, the target cell is a healthy cell. In some embodiments, the target cell is not associated with a disease or disorder. In some embodiments, the target cell is unstimulated. In some embodiments, the target cell is not activated. In some embodiments, the target cell is a stimulated cell. In some embodiments, the target cell is activated.

In some embodiments, the target cell is a cell associated with a disease or disorder. In some embodiments, disease or disorder is a cancer, an autoimmune disease, an infectious disease, a metabolic disease, a neurodegenerative disease, or a genetic disease (e.g., enzyme deficiency).

In some embodiments, the disease or disorder is a cancer. In some embodiments, the cancer is selected from leukemia, polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia, myelodysplasia, sarcoma, carcinoma, fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancer, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyngioma, ependymoma, pinealoma, hemangioblastoma, acoustic neuroma, oligodendroglioma, meningioma, melanoma, neuroblastoma and retinoblastoma).

In some embodiments, the disease or disorder is an autoimmune disease or disorder. In some embodiments, the disease or disorder is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Graves disease, Crohn's disease, multiple sclerosis, asthma, and/or a disease or condition associated with transplant.

In some embodiments, the autoimmune or inflammatory disorder is selected from chronic graft-vs-host disease (GVHD), lupus, arthritis, immune complex glomerulonephritis, goodpasture, uveitis, hepatitis, systemic sclerosis or scleroderma, type I diabetes, multiple sclerosis, cold agglutinin disease, Pemphigus vulgaris, Grave's disease, autoimmune hemolytic anemia, Hemophilia A, Primary Sjogren's Syndrome, thrombotic thrombocytopenia purrpura, neuromyelits optica, Evan's syndrome, IgM mediated neuropathy, cyroglobulinemia, dermatomyositis, idiopathic thrombocytopenia, ankylosing spondylitis, bullous pemphigoid, acquired angioedema, chronic urticarial, antiphospholipid demyelinating polyneuropathy, and autoimmune thrombocytopenia or neutropenia or pure red cell aplasias, while exemplary non-limiting examples of alloimmune diseases include allosensitization (see, for example, Blazar et al., 2015, Am. J. Transplant, 15(4):931-41) or xenosensitization from hematopoietic or solid organ transplantation, blood transfusions, pregnancy with fetal allosensitization, neonatal alloimmune thrombocytopenia, hemolytic disease of the newborn, sensitization to foreign antigens such as can occur with replacement of inherited or acquired deficiency disorders treated with enzyme or protein replacement therapy, blood products, and gene therapy. In some embodiments, the antigen characteristic of an autoimmune or inflammatory disorder is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor.

In some embodiments, the target is a subcellular component. In some embodiments, the subcellular component is a cytoplasmic component. In some embodiments, the subcellular component is a nucleus, ribosome, an endoplasmic reticulum, a Golgi apparatus, a cytoskeleton, a mitochondria, a vacuole and a vesicle. In some embodiments, the subcellular component is a nucleus, ribosome, an endoplasmic reticulum, a Golgi apparatus, a cytoskeleton, a mitochondria, a vacuole or a vesicle. In some embodiments, the subcellular component is a nucleus. In some embodiments, the subcellular component is isolated. In some embodiments, the subcellular component is isolated using any isolation method useful and known to one of skill in the art. In some embodiments, the subcellular component is not isolated.

### II. Oligonucleotides

In some embodiments, all or a portion of any of the compositions provided herein is or contains an oligonucleotide. In some embodiments, all or a portion of any of the complexes provided herein is or contains an oligonucleotide. In some embodiments, the oligonucleotide is a polymeric sequence. In some embodiments, an oligonucleotide is a single-stranded multimer of nucleotides from about 2 to about 500 nucleotides in length. In some embodiments, any of the oligonucleotides described herein can be synthetic, made enzymatically (e.g., via polymerization), or using a “split-pool” method. In some embodiments, any of the oligonucleotides described herein can include ribonucleotide monomers (i.e., can be oligoribonucleotides) and/or deoxyribonucleotide monomers (i.e., oligodeoxyribonucleotides). In some embodiments, any of the oligonucleotides described herein can include a combination of both deoxyribonucleotide monomers and ribonucleotide monomers in the oligonucleotide (e.g., random or ordered combination of deoxyribonucleotide monomers and ribonucleotide monomers). In some embodiments, the oligonucleotide can be 4 to 10, 11 to 20, 21 to 30, 31 to 40, 41 to 50, 51 to 60, 61 to 70, 71 to 80, 81 to 100, 101 to 150, 151 to 200, 201 to 250, 251 to 300, 301 to 350, 351 to 400, or 401-500 nucleotides in length. In some embodiments, any of the oligonucleotides described herein can include one or more functional moieties that are attached (e.g., covalently or non-covalently) to another structure. In some embodiments, any of the oligonucleotides described herein can include one or more detectable labels (e.g., a radioisotope or fluorophore).

In some of any embodiments, the oligonucleotide comprises an anchor or anchor sequence. In some embodiments, the oligonucleotide comprises a labeling oligonucleotide. In some embodiments, the labeling oligonucleotide comprises a binding site for a primer, a sample barcode sequence and a capture sequence.

### A. Universal Anchor

In some of any embodiments, any of the compositions described herein contain a universal anchor. In some of any embodiments, any of the complexes described herein contain a universal anchor. In some of any embodiments, the universal anchor is conjugated to any of the glycan-binding agents described herein via any of the conjugation methods described herein. In some embodiments, the universal anchor is an anchor that is substantially identical to all other anchors in a composition. In some embodiments, the universal anchor is an anchor that is substantially identical to all other anchors in a first and or one or more additional compositions. In some embodiments, the universal anchor is an anchor containing an oligonucleotide or oligonucleotide sequence. In some embodiments, the universal anchor is an anchor containing an oligonucleotide or oligonucleotide sequence that is substantially identical to all other anchors containing an oligonucleotide or oligonucleotide sequence in a composition. In some embodiments, the universal anchor contains a region of nucleotides that is common to two or more nucleic acid molecules. In some embodiments, any of the universal anchors described herein is an oligonucleotide sequence that is also a hybridization handle. In some embodiments, the universal anchor is a polynucleotide or oligonucleotide sequence, which is designed to hybridize to a complementary oligonucleotide sequence. In some embodiments, the universal anchor reversibly hybridizes to a complementary oligonucleotide sequence. In some embodiments, the universal anchor contains a region of nucleotides that allow for hybridization to multiple different complementary sequences. In some embodiments, the universal anchor reversibly hybridizes to a first complementary oligonucleotide sequence on a first labeling oligonucleotide. In some embodiments, the universal anchor reversibly hybridizes to a second complementary oligonucleotide sequence on a second labeling oligonucleotide. In some embodiments, the universal anchor is designed for the purpose of generating a double stranded construct oligonucleotide sequence. In some embodiments, the universal anchor may be present in different members of a plurality of nucleic acid molecules or target sequences and can allow the replication or amplification of multiple different sequences using a single universal primer that is complementary to the universal sequence. In some embodiments, each universal anchor in the first plurality of complexes in any of the compositions provided herein contains the same sequence. In some embodiments, each universal anchor hybridizes to a substantially similar complementary sequence. In other embodiments, each universal anchor of the one or more additional pluralities of complexes contains the same sequence. In some embodiments, each universal anchor in the first plurality of complexes in any of the compositions provided herein contains a distinct or different sequence. In some embodiments, each universal anchor hybridizes to a substantially different complementary sequence. In other embodiments, each universal anchor of the one or more additional pluralities of complexes contains a distinct or different sequence.

In some embodiments, each universal anchor contains an amine-modified oligonucleotide sequence. In some embodiments, the universal anchor is a 5′-azide modified oligonucleotide. In some embodiments, 5-Aminohexylacrylamido-dUTP (aha-dUTP) and 5-aminohexylacrylamido-dCTP (aha-dCTP) can be used to produce amine-modified oligonucleotide sequences. In some embodiments, the universal anchor is modified by enzymatic incorporation methods including but not limited to reverse transcription, nick translation, random primed labeling or PCR. An amine-modified oligonucleotide sequence can be a sequence that further contains random hexamer oligonucleotides. An amine-modified oligonucleotide sequence can be a sequence that is labeled with any amine-reactive dye or hapten. In some embodiments, each universal anchor is or contains a locked nucleic acid (LNA). A locked nucleic acid (LNA) may be a modified RNA monomer. In some embodiments, the LNA contains a methylene bridge bond linking a 2′ oxygen to a 4′ carbon of an RNA pentose ring. In some embodiments, the bridge bond fixes the pentose ring in the 3′-endo conformation. In some embodiments, the universal anchor containing a LNA has heightened structural stability, increased hybridization melting temperature, and increased resistance to nucleases. In some embodiments, each universal anchor of the first plurality of complexes comprises at least 3 or more nucleotide bases which hybridize to a complementary sequence on a labeling oligonucleotide. In some embodiments, each universal anchor of the first plurality of complexes comprises at least 3 or more nucleotide bases which reversibly hybridize to a complementary sequence on a labeling oligonucleotide. In some embodiments, reversibly hybridized may mean non-covalently hybridized. In some embodiments, each universal anchor of the one or more additional pluralities of complexes comprises at least 3 or more nucleotide bases which hybridize to a complementary sequence on a labeling oligonucleotide. In some embodiments, each universal anchor of the one or more additional pluralities of complexes comprises at least 3 or more nucleotide bases which reversibly hybridize to a complementary sequence on a labeling oligonucleotide. In some embodiments, the universal anchor comprises 3, 4, 5, 6, 7, 8, 9, 10, 11, 12, 13, 14, 15, 16, 17, 18, 19 or 20 nucleotide bases.

In some embodiments, the universal anchor is conjugated to any of the glycan binding agents provided herein. Various conjugation methods are capable of being used in conjugating the universal anchor to the glycan binding agent. In some embodiments, the universal anchor is conjugated to the glycan binding agent by ionic interactions, affinity conjugation or direct conjugation. In some embodiments, the universal anchor is conjugated to the glycan binding agent by click-chemistry or droplet-based chemistry. In some embodiments, the universal anchor is conjugated to the glycan binding agent by a DBCO-azide click or stain-promoted azide-alkyne cycloaddition (SPAAC) reaction. In some embodiments, the universal anchor is non-covalently (i.e. reversibly) bound to any of the glycan-binding agents provided herein. In some embodiments, the universal anchor is covalently (i.e. non-reversibly) bound to any of the glycan-binding agents provided herein.

### B. Labeling Oligonucleotide

Provided herein are labeling oligonucleotides that contain a complementary sequence that hybridizes to a universal anchor. Provided herein are labeling oligonucleotides that do not contain a complementary sequence that hybridizes to a universal anchor. In some embodiments, the labeling oligonucleotide hybridizes to a complementary sequence on a universal anchor via a binding site for a primer. In some embodiments, the labeling oligonucleotide is conjugated directly to any of the glycan-binding agents described herein by any of the conjugation methods described herein. In some embodiments, the labeling oligonucleotide is non-covalently (i.e. reversibly) bound to any of the universal anchors provided herein. In some embodiments, the labeling oligonucleotide is covalently (i.e. non-reversibly) bound to any of the universal anchors provided herein. In some embodiments, the labeling oligonucleotide is non-covalently (i.e. reversibly) bound to any of the glycan-binding agents provided herein. In some embodiments, the labeling oligonucleotide is covalently (i.e. non-reversibly) bound to any of the glycan-binding agents provided herein. In some embodiments, the labeling oligonucleotide can be a first labeling oligonucleotide that is reversibly hybridized to a universal anchor. In some embodiments, the labeling oligonucleotide can be a second labeling oligonucleotide that is reversibly hybridized to the universal anchor. In some embodiments, the labeling oligonucleotide can be one or more additional labeling oligonucleotides that are reversibly hybridized to the universal anchor. In some embodiments, the one or more additional labeling oligonucleotides can be 1, 2, 3, 4, 5, 6, 7, 8, 9 or 10 or more labeling oligonucleotides. In some embodiments, the one or more additional labeling oligonucleotides can be between 1 and 100 labeling oligonucleotides. In some embodiments, the one or more additional labeling oligonucleotides can be 100 or more additional labeling oligonucleotides.

In some embodiments, any of the labeling oligonucleotides provided herein can contain a binding site for a primer, a sample barcode sequence and a capture sequence. In some embodiments, the labeling oligonucleotide further contains a detectable label. In some embodiments, the labeling oligonucleotide further hybridizes to a complementary sequence on a capture polymer. In some embodiments, the labeling oligonucleotide hybridizes to a complementary sequence on a capture polymer via the capture sequence.

i. Binding Site for a Primer

In some embodiments, the binding site for a primer is a functional component of the labeling oligonucleotide which is also an oligonucleotide or polynucleotide sequence that provides an annealing site for amplification of the labeling oligonucleotide sequence. In some embodiments, the binding site for the primer is also a hybridization site for the universal anchor. In some embodiments, the binding site for a primer contains a sequence of nucleotides that are complementary to the universal anchor. The binding site for a primer can be formed of polymers of DNA, RNA, PNA, modified bases or combinations of these bases, or polyamides, etc. In some embodiments, the binding site for a primer is about 10 of such monomeric components, e.g., nucleotide bases, in length. In other embodiments, the binding site for a primer is at least about 5 to 100 monomeric components, e.g., nucleotides, in length. Thus in various embodiments, the binding site for a primer is formed of a sequence of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99 or up to 100 monomeric components, e.g., nucleic acids. In some embodiments, the binding site for a primer can be the same or different, depending upon the techniques intended to be used for amplification. In certain embodiments, the binding site for a primer can be a generic sequence suitable as a annealing site for a variety of amplification technologies. Amplification technologies include, but are not limited to, DNA-polymerase based amplification systems, such as polymerase chain reaction (PCR), real-time PCR, loop mediated isothermal amplification (LAMP, MALBAC), strand displacement amplification (SDA), multiple displacement amplification (MDA), recombinase polymerase amplification (RPA) and polymerization by any number of DNA polymerases (for example, T4 DNA polymerase, Sulfulobus DNA polymerase, Klenow DNA polymerase, Bst polymerase, Phi29 polymerase) and RNA-polymerase based amplification systems (such as T7-, T3-, and SP6-RNA-polymerase amplification), nucleic acid sequence based amplification (NASBA), self-sustained sequence replication (3SR), rolling circle amplification (RCA), ligase chain reaction (LCR), helicase dependent amplification (I), ramification amplification method and RNA-seq.

In some embodiments, a primer is a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers may also include both RNA nucleotides and DNA nucleotides (e.g., in a random or designed pattern). Primers may also include other natural or synthetic nucleotides described herein that can have additional functionality. In some examples, DNA primers can be used to prime RNA synthesis and vice versa (e.g., RNA primers can be used to prime DNA synthesis). Primers can vary in length. In some embodiments, primers can be about 6 bases to about 120 bases. In some embodiments, primers can include up to about 25 bases. In some of any embodiments, a primer is a primer binding sequence.

In some embodiments, the use of a primer includes primer extension. In some embodiments, the primer extension is or involves a method where two nucleic acid sequences (e.g., a constant region from each of two distinct capture probes) become linked (e.g., hybridized) by an overlap of their respective terminal complementary nucleic acid sequences (i.e., for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, the use of a primer includes nucleic acid extension. In some embodiments, nucleic acid extension involves incorporation of one or more nucleic acids (e.g., A, G, C, T, U, nucleotide analogs, or derivatives thereof) into a molecule (such as, but not limited to, a nucleic acid sequence) in a template-dependent manner, such that consecutive nucleic acids are incorporated by an enzyme (such as a polymerase or reverse transcriptase), thereby generating a newly synthesized nucleic acid molecule. For example, a primer that hybridizes to a complementary nucleic acid sequence can be used to synthesize a new nucleic acid molecule by using the complementary nucleic acid sequence as a template for nucleic acid synthesis. Similarly, a 3′ polyadenylated tail of an mRNA transcript that hybridizes to a poly (dT) sequence (e.g., capture domain) can be used as a template for single-strand synthesis of a corresponding cDNA molecule.

In some embodiments, the use of a primer includes amplification. In some embodiments, amplification encompasses generating copies of genetic material, including DNA and RNA sequences. In some embodiments, during amplification the reaction mixture includes the genetic material to be amplified, an enzyme, one or more primers that are employed in a primer extension reaction, and reagents for the reaction. The oligonucleotide primers are of sufficient length to provide for hybridization to complementary genetic material under annealing conditions. The length of the primers generally depends on the length of the amplification domains, but will typically be at least 4 bases, at least 5 bases, at least 6 bases, at least 8 bases, at least 9 bases, at least 10 base pairs (bp), at least 11 bp, at least 12 bp, at least 13 bp, at least 14 bp, at least 15 bp, at least 16 bp, at least 17 bp, at least 18 bp, at least 19 bp, at least 20 bp, at least 25 bp, at least 30 bp, at least 35 bp, and can be as long as 40 bp or longer, where the length of the primers will generally range from 18 to 50 bp. The genetic material can be contacted with a single primer or a set of two primers (forward and reverse primers), depending upon whether primer extension, linear or exponential amplification of the genetic material is desired.

In some embodiments, the amplification process uses a DNA polymerase enzyme. The DNA polymerase activity can be provided by one or more distinct DNA polymerase enzymes. In certain embodiments, the DNA polymerase enzyme is from a bacterium, e.g., the DNA polymerase enzyme is a bacterial DNA polymerase enzyme. For instance, the DNA polymerase can be from a bacterium of the genus Escherichia, Bacillus, Thermophilus, or Pyrococcus. Suitable examples of DNA polymerases that can be used include, but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

In some embodiments, a DNA polymerase includes not only naturally-occurring enzymes but also all modified derivatives thereof, including derivatives of naturally-occurring DNA polymerase enzymes. In some embodiments, the DNA polymerase can be modified to remove 5′-3′ exonuclease activity. Sequence-modified derivatives or mutants of DNA polymerase enzymes that can be used include, but are not limited to, mutants that retain at least some of the functional, e.g. DNA polymerase activity of the wild-type sequence. Mutations can affect the activity profile of the enzymes, e.g. enhance or reduce the rate of polymerization, under different reaction conditions, e.g. temperature, template concentration, primer concentration, etc. Mutations or sequence-modifications can also affect the exonuclease activity and/or thermostability of the enzyme.

In some embodiments, amplification can include reactions such as, but not limited to, a strand-displacement amplification reaction, a rolling circle amplification reaction, a ligase chain reaction, a transcription-mediated amplification reaction, an isothermal amplification reaction, and/or a loop-mediated amplification reaction. In some embodiments, amplification uses a single primer that is complementary to the 3′ tag of target DNA fragments. In some embodiments, amplification uses a first and a second primer, where at least a 3′ end portion of the first primer is complementary to at least a portion of the 3′ tag of the target nucleic acid fragments, and where at least a 3′ end portion of the second primer exhibits the sequence of at least a portion of the 5′ tag of the target nucleic acid fragments. In some embodiments, a 5′ end portion of the first primer is non-complementary to the 3′ tag of the target nucleic acid fragments, and a 5′ end portion of the second primer does not exhibit the sequence of at least a portion of the 5′ tag of the target nucleic acid fragments. In some embodiments, the first primer includes a first universal sequence and/or the second primer includes a second universal sequence. In some embodiments (e.g., when the amplification amplifies captured DNA), the amplification products can be ligated to additional sequences using a DNA ligase enzyme. The DNA ligase activity can be provided by one or more distinct DNA ligase enzymes. In some embodiments, the DNA ligase enzyme is from a bacterium, e.g., the DNA ligase enzyme is a bacterial DNA ligase enzyme. In some embodiments, the DNA ligase enzyme is from a virus (e.g., a bacteriophage). For instance, the DNA ligase can be T4 DNA ligase. Other enzymes appropriate for the ligation step include, but are not limited to, Tth DNA ligase, Taq DNA ligase, Thermococcus sp. (strain 9oN) DNA ligase (9oN™ DNA ligase, available from New England Biolabs, Ipswich, Mass.), and Ampligase™ (available from Epicentre Biotechnologies, Madison, Wis.). Derivatives, e.g. sequence-modified derivatives, and/or mutants thereof, can also be used.

In some embodiments, genetic material is amplified by reverse transcription polymerase chain reaction (RT-PCR). The desired reverse transcriptase activity can be provided by one or more distinct reverse transcriptase enzymes, suitable examples of which include, but are not limited to: M-MLV, MuLV, AMV, HIV, ArrayScript™, MultiScribe™ ThermoScript™, and SuperScript® I, II, III, and IV enzymes. “Reverse transcriptase” includes not only naturally occurring enzymes, but all such modified derivatives thereof, including also derivatives of naturally-occurring reverse transcriptase enzymes. In addition, reverse transcription can be performed using sequence-modified derivatives or mutants of M-MLV, MuLV, AMV, and HIV reverse transcriptase enzymes, including mutants that retain at least some of the functional, e.g. reverse transcriptase, activity of the wild-type sequence. The reverse transcriptase enzyme can be provided as part of a composition that includes other components, e.g. stabilizing components that enhance or improve the activity of the reverse transcriptase enzyme, such as Rnase inhibitor(s), inhibitors of DNA-dependent DNA synthesis, e.g. actinomycin D. Many sequence-modified derivative or mutants of reverse transcriptase enzymes, e.g. M-MLV, and compositions including unmodified and modified enzymes are commercially available, e.g. ArrayScript™, MultiScribe™, ThermoScript™ and SuperScript® I, II, III, and IV enzymes.

Certain reverse transcriptase enzymes (e.g. Avian Myeloblastosis Virus (AMV) Reverse Transcriptase and Moloney Murine Leukemia Virus (M-MuLV, MMLV) Reverse Transcriptase) can synthesize a complementary DNA strand using both RNA (cDNA synthesis) and single-stranded DNA (ssDNA) as a template. Thus, in some embodiments, the reverse transcription reaction can use an enzyme (reverse transcriptase) that is capable of using both RNA and ssDNA as the template for an extension reaction, e.g. an AMV or MMLV reverse transcriptase. In some embodiments, the quantification of RNA and/or DNA is carried out by real-time PCR (also known as quantitative PCR or qPCR), using techniques well known in the art, such as but not limited to “TAQMAN™” or “SYBR®”, or on capillaries (“LightCycler® Capillaries”). In some embodiments, the quantification of genetic material is determined by optical absorbance and with real-time PCR. In some embodiments, the quantification of genetic material is determined by digital PCR. In some embodiments, the genes analyzed can be compared to a reference nucleic acid extract (DNA and RNA) corresponding to the expression (mRNA) and quantity (DNA) in order to compare expression levels of the target nucleic acids.

ii. Sample Barcode Sequence

In some of any embodiments, the labeling oligonucleotide contains a sample barcode sequence. In some embodiments, the terms “sample barcode sequence” and “barcode” are used interchangeably. In some embodiments, a sample barcode sequence can include polynucleotides, polynucleotide barcodes, random nucleic acids and/or amino acid sequences, and synthetic nucleic acid and/or amino acid sequences. In some embodiments, a sample barcode sequence is or contains a polymer, e.g., a polynucleotide, which when it is a functional element of the labeling oligonucleotide, is specific for a single ligand. As used in the various methods described herein, the sample barcode sequence can be a “cell barcode”, a “substrate barcode”, and/or a “spatial barcode”, which describes a defined polynucleotide, specific for identifying a particular cell, cellular component, ligand or substrate, e.g., Drop-seq microbead, 10× bead, or location of a cellular component on a tissue sample. In some embodiments, the sample barcode sequence can act as a unique identifier of a specific cell. In some embodiments, the sample barcode sequence can act as a unique identifier of a subset of cells. In some embodiments, the sample barcode sequence can spatially resolve cellular or molecular components found in a biological sample, which can be at single cell resolution. In some embodiments, the sample barcode sequence can be formed of a defined sequence of DNA, RNA, modified bases or combinations of these bases. In some embodiments, the sample barcode sequence is about 2 to 4 monomeric components, e.g., nucleotide bases, in length. In other embodiments, the sample barcode sequence is at least about 1 to 100 monomeric components, e.g., nucleotides, in length. Thus in various embodiments, the sample barcode sequence is formed by a sequence of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99 or up to 100 monomeric components, e.g., nucleic acids.

In some embodiments, any of the compositions or methods described herein may utilize a plurality of labeling oligonucleotides, where some or all of the labeling oligonucleotides comprise a different sample barcode sequence. In some embodiments, any of the compositions or methods described herein may utilize a plurality of labeling oligonucleotides, where some or all of the labeling oligonucleotides comprise a substantially identical sample barcode sequence. In some embodiments, any of the compositions or methods described herein may utilize a plurality of labeling oligonucleotides, where some or all of the labeling oligonucleotides comprise the same sample barcode sequence. Exemplary sample barcode sequences can be found in, but are not limited to, SEQ ID NOs: 6-13.

iii. Capture Sequence and Capture Polymer

In some of any embodiments, the labeling oligonucleotide can contain a capture sequence. In certain embodiments, the capture sequence can be formed of a sequence of monomers of a selected polymer, e.g., DNA, RNA, modified bases or combinations of these bases, PNAs, polyamides, etc. In some embodiments, an capture sequence is about 3 to 15 monomeric components, e.g., nucleotides, in length. In other embodiments, each capture sequence can be at least about 3 to 100 monomeric components, e.g., nucleotides, in length. Thus in various embodiments, the capture sequence is formed of a sequence of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99 or up to 100 monomeric components, e.g., nucleic acids.

In some embodiments, any of the compositions or methods described herein may utilize a plurality of labeling oligonucleotides, where some or all of the labeling oligonucleotides comprise a different capture sequence. In some embodiments, any of the compositions or methods described herein may utilize a plurality of labeling oligonucleotides, where some or all of the labeling oligonucleotides comprise the same capture sequence. In some embodiments, any of the compositions or methods described herein may utilize a plurality of labeling oligonucleotides, where some or all of the labeling oligonucleotides comprise an capture sequence that is substantially identical.

In some embodiments, the capture sequence may hybridize to a free complementary sequence or with a complementary sequence that is immobilized on a substrate. In some embodiments, an capture sequence is a polyA sequence. In other embodiments, an capture sequence is a polyT sequence. In still other embodiments, the capture sequence may be a random sequence provided that it can hybridize to its intended complementary sequence.

In some of any embodiments, the capture sequence comprises or consists of a polyA sequence. In certain embodiments, a polyA sequence comprises a nucleic acid sequence comprising ten or more (e.g., 10-40, 10-30 or 10-20) consecutive adenosine nucleotides, derivatives or variants of an adenosine nucleotide, the like, or a combination thereof. In some of any embodiments, the capture sequence comprises or consists of a polyT sequence. In some of any embodiments, the capture sequence comprises or consists of a polyG sequence. In some of any embodiments, the capture sequence comprises or consists of a random sequence provided that it can hybridize to its intended complementary sequence (e.g., a capture oligonucleotide, amplification primer, or the like). In some embodiments, the polyA sequence of a plurality of capture sequences is substantially identical. As understood by one of skill in the art, polyA sequences that are substantially identical may differ substantially in length. In some embodiments, a polyA sequence (e.g, a polyA sequence of an anchor) is a nucleic acid configured to hybridize to a polyT sequence (e.g., an oligonucleotide or capture oligonucleotide comprising a polyT sequence). As understood by one of skill in the art, depending on hybridization conditions a polyA sequence may comprise one, two, three or four non-polyA nucleotides and still hybridize efficiently to a polyT sequence, thereby providing an annealed polyA-polyT complex comprising one, two, three or more mismatches. Accordingly, in some embodiments, a polyA sequence is a nucleic acid sequence comprising at least 70%, at least 75%, at least 80%, at least 85%, at least 90%, at least 95% or 100% adenosine nucleotides, adenosine analogs, adenosine variants or a combination thereof. Depending upon the assay steps involved and the intended target, the capture sequence can be unhindered or “free” in the biological sample.

In some embodiments, a capture polymer is a polymeric sequence, e.g., an oligonucleotide, comprising at least a sequence that is complementary to the capture sequence. In some embodiments, the capture polymer is not part of the labeling oligonucleotide. In some embodiments, the capture polymer is a free complementary sequence. In some embodiments, the capture polymer is any polymeric sequence or oligonucleotide belonging to a construct-purification kit or an mRNA-sequencing kit. As used herein, the capture polymer contains a complementary sequence to which a capture sequence is intended to hybridize to, often resulting in a hybridized double stranded complex. In the presence of a polymerase, a hybridized complex can often be extended in a 3′ direction where a nucleic acid template is present. Accordingly, in certain embodiments, a sequence complementary to a capture sequence can hybridize to a capture polymer. In some of any embodiments, the capture polymer and its complementary sequence can be formed of DNA, RNA, modified bases or combinations of these bases or of any other polymeric component as defined above. In some embodiments, the capture polymer contains a complementary sequence that is a primer sequence designed to participate in amplifying the capture sequence. In some embodiments, the capture polymer is immobilized on a substrate. In some embodiments, the solid substrate is a bead, a microfluidics bead, a slide, a multi-well plate or a chip. Similar to the capture sequence, each capture polymer can be at least about 3 to about 100 monomeric units, e.g., nucleotides, in length. Thus in various embodiments, the capture polymer or its complementary sequence is formed of a sequence of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99 or up to 100 monomeric units, e.g., nucleic acids. In some embodiments, a capture polymer contains a complementary polyT sequence when the capture sequence is a polyA sequence. In another embodiment, the capture polymer contains a polyA sequence. In still another embodiment, the complementary sequence may be a random polymer, e.g., oligonucleotide sequence, provided that it can hybridize to its intended capture sequence.

### C. Detectable Label

In some embodiments, any of the compositions provided herein may contain a detectable label. In some embodiments, the detectable label is or comprises a detectable moiety. In some embodiments, the detectable label is or comprises a tag or fluorescent label. In some embodiments, the detectable label is associated with (e.g. conjugated to) the universal anchor. In some embodiments, the detectable label is associated with (e.g. conjugated to) the labeling oligonucleotide. In some embodiments, the detectable label is associated with (e.g. conjugated to) the binding site for a primer on the labeling oligonucleotide. In some embodiments, the detectable label is associated with (e.g. conjugated to) the sample barcode sequence on the labeling oligonucleotide. In some embodiments, the detectable label is associated with (e.g. conjugated to) the capture sequence on the labeling oligonucleotide. In some of any embodiments, the detectable label is reversibly associated (non-covalently associated). In some embodiments, the detectable label is not reversibly associated (covalently associated).

In some embodiments, the detectable label is or comprises a detectable moiety that is associated with (e.g., conjugated to) a molecule to be detected, e.g., a probe for in situ assay, a capture probe or analyte. The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

In some of any embodiments, the detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some embodiments, the detectable label is bound to a feature or to a capture probe associated with a feature. For example, detectably labeled features can include a fluorescent, a colorimetric, or a chemiluminescent label attached to a bead (see, for example, Rajeswari et al., J. Microbiol Methods 139:22-28, 2017, and Forcucci et al., J. Biomed Opt. 10:105010, 2015, the entire contents of each of which are incorporated herein by reference). In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor®350, Alexa Fluor®430, Alexa Fluor® 488, Alexa Fluor®532, Alexa Fluor®546, Alexa Fluor®555, Alexa Fluor®568, Alexa Fluor®594, Alexa Fluor®633, Alexa Fluor®647, Alexa Fluor®660, Alexa Fluor®680, Alexa Fluor®700, Alexa Fluor®750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™ Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy2, Cy3®, Cy3.5, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilC18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker@Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™_1, POPO™-3/PO-PRO™_3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed, SYTO 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rhol01, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

In some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate substrates (e.g., an oxidizing reagent plus a chemiluminescent compound. A number of compound families are known to provide chemiluminescence under a variety of conditions. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and -methoxy substituents, oxalates such as oxalyl active esters, p-nitrophenyl, N-alkyl acridinum esters, luciferins, lucigenins, or acridinium esters. In some embodiments, a detectable label is or includes a metal-based or mass-based label. For example, small cluster metal ions, metals, or semiconductors may act as a mass code. In some examples, the metals can be selected from Groups 3-15 of the periodic table, e.g., Y, La, Ag, Au, Pt, Ni, Pd, Rh, Ir, Co, Cu, Bi, or a combination thereof.

### D. Conjugation Methods

Provided herein are methods of conjugating moieties to any of the compositions or complexes described herein. In some embodiments, the methods include conjugating a universal anchor to any of the compositions or complexes described herein. In some embodiments, the methods include conjugating a universal anchor to any of the glycan-binding agents described herein. In some embodiments, the methods include conjugating a labeling oligonucleotide to any of the compositions or complexes provided herein. In some embodiments, the methods include conjugating a labeling oligonucleotide to any of the glycan-binding agents described herein.

In some embodiments, the conjugation method is a thiol-based conjugation method. In some embodiments, the conjugation method is not a thiol-based conjugation method. In some embodiments, the conjugation method involves conjugation to a lysine side amino group. In some embodiments, the conjugation to a lysine side amino group includes the presence of a ligand. In some embodiments, the conjugation method involves the use of an IgG glycan as a site for oxime and hydrazine bioconjugation. In some embodiments, the conjugation method involves the oxidation of vicinal hydroxyl groups on the carbohydrate chain of glycoproteins followed by reaction with an aminooxy- or hydrazide-based moiety. In some embodiments, the oxidation step can be chemical based, e.g. using periodate ions, or can be enzymatic based, e.g. using galactose oxidase.

### III. Complexes

Provided herein are compositions and methods which contain complexes comprising any of the glycan binding agents described herein. In some embodiments, the complexes provided herein comprise a glycan-binding agent conjugated to a universal anchor. In some embodiments, the complexes provided herein comprise a glycan-binding agent conjugated to a universal anchor, further hybridized to a labeling oligonucleotide. In some embodiments, the complexes provided herein comprise a glycan-binding agent conjugated to a universal anchor, further hybridized to a labeling oligonucleotide comprising a binding site for a primer, a sample barcode sequence and a capture sequence. In some embodiments, any of the compositions or methods described herein is or contain a first plurality of complexes. In some embodiments, any of the compositions or methods described herein is or contain one or more additional pluralities of complexes.

a. First Plurality

Provided herein are compositions and methods which are or contain a first plurality of complexes. In some embodiments, the first plurality of complexes is or contains a glycan binding agent. In some embodiments, the first plurality of complexes provided herein is or contains a glycan-binding agent conjugated to a universal anchor.

In some embodiments, each complex of the first plurality of complexes contains a substantially identical glycan-binding agent. In some embodiments, each complex of the first plurality of complexes contains a distinct glycan-binding agent. In some embodiments, each complex of the first plurality of complexes contains a substantially identical glycan-binding agent conjugated to a universal anchor. In some embodiments, each complex of the first plurality of complexes contains a distinct glycan-binding agent conjugated to a universal anchor. In some embodiments, the glycan-binding agent of the first plurality is selected from concanavalin A (ConA), a wheat germ agglutinin (WGA), a pea lectin, a soybean lectin, a lentil lectin, a ricin, a bacterial toxin, an influenza virus hemagglutinin, a C-type lectin, an S-type lectin, a P-type lectin, or an I-type lectin. In some embodiments, glycan-binding agent of the first plurality is concanavalin A (ConA). In some embodiments, glycan-binding agent of the first plurality is wheat-germ agglutinin (WGA).

In some embodiments, the first plurality of complexes is or contains 2 or more glycan-binding agents conjugated to a universal anchor. In some embodiments, the first plurality of complexes is or contains between 1 to 100 cell glycan-binding agents conjugated to a universal anchor. In some embodiments, the first plurality of complexes is or contains 100 or more glycan-binding agents conjugated to a universal anchor. In some embodiments, the first plurality of complexes is or contains 1000 or more glycan-binding agents conjugated to a universal anchor.

In some embodiments, the first plurality of complexes is or contains 2 or more glycan-binding agents conjugated to a labeling oligonucleotide. In some embodiments, the first plurality of complexes is or contains between 1 to 100 cell glycan-binding agents conjugated to a labeling oligonucleotide. In some embodiments, the first plurality of complexes is or contains 100 or more glycan-binding agents conjugated to a labeling oligonucleotide. In some embodiments, the first plurality of complexes is or contains 1000 or more glycan-binding agents conjugated to a labeling oligonucleotide.

In some of any embodiments, the first plurality of complexes contains a substantially identical glycan-binding agent conjugated to a universal anchor, which is hybridized to a labeling oligonucleotide. In some of any embodiments, the first plurality of complexes contains a substantially identical glycan-binding agent conjugated to a universal anchor, which is hybridized to a labeling oligonucleotide which contains a binding site for a primer, a sample barcode sequence and a capture sequence. In some of any embodiments, the first plurality of complexes contains a substantially identical glycan-binding agent conjugated to a universal anchor, which is hybridized to a labeling oligonucleotide which contains a binding site for a primer, a sample barcode sequence, a capture sequence and a detectable label.

In some of any embodiments, the first plurality of complexes contains a substantially identical glycan-binding agent conjugated to a universal anchor, which is hybridized to a complementary sequence on a labeling oligonucleotide. In some of any embodiments, the first plurality of complexes contains a substantially identical glycan-binding agent conjugated to a universal anchor, which is hybridized to a complementary sequence on a binding site for a primer on a labeling oligonucleotide.

In some of any embodiments, the first plurality of complexes contains a distinct glycan-binding agent conjugated to a universal anchor, which is hybridized to a labeling oligonucleotide. In some of any embodiments, the first plurality of complexes contains a distinct glycan-binding agent conjugated to a labeling oligonucleotide. In some of any embodiments, the first plurality of complexes contains a distinct glycan-binding agent conjugated to a universal anchor, which is hybridized to a labeling oligonucleotide which contains a binding site for a primer, a sample barcode sequence and a capture sequence. In some of any embodiments, the first plurality of complexes contains a distinct glycan-binding agent conjugated to a universal anchor, which is hybridized to a labeling oligonucleotide which contains a binding site for a primer, a sample barcode sequence, a capture sequence and a detectable label. In some of any embodiments, the first plurality of complexes contains a distinct glycan-binding agent conjugated to a universal anchor, which is hybridized to a complementary sequence on a labeling oligonucleotide. In some of any embodiments, the first plurality of complexes contains a distinct glycan-binding agent conjugated to a universal anchor, which is hybridized to a complementary sequence on a binding site for a primer on a labeling oligonucleotide.

In some embodiments, each of the first plurality of complexes is hybridized or conjugated to a labeling oligonucleotide which contains a substantially identical binding site for a primer. In some embodiments, each complex of the first plurality of complexes is hybridized to a labeling oligonucleotide which contains a distinct binding site for a primer. In some embodiments, each complex of the first plurality of complexes is hybridized to a labeling oligonucleotide which contains a substantially identical sample barcode sequence. In some embodiments, each complex of the first plurality of complexes is hybridized to a labeling oligonucleotide which contains a distinct sample barcode sequence. In some embodiments, each complex of the first plurality of complexes is hybridized to a labeling oligonucleotide which contains a substantially identical capture sequence. In some embodiments, each complex of the first plurality of complexes is hybridized to a labeling oligonucleotide which contains a distinct sample capture sequence.

In some embodiments, each sample barcode sequence within the first plurality comprises a substantially identical binding site for a primer. In some embodiments, each sample barcode sequence within the first plurality comprises a different binding site for a primer. In some embodiments, each sample barcode sequence within the first plurality comprises a substantially identical barcode sequence. In some embodiments, each sample barcode sequence within the first plurality comprises a different barcode sequence. In some embodiments, each sample barcode sequence within the first plurality comprises a substantially identical capture sequence. In some embodiments, each sample barcode sequence within the first plurality comprises a different capture sequence.

In some embodiments, each complex of the first plurality of complexes hybridized to a labeling oligonucleotide further hybridizes to a complementary sequence on a capture polymer. In some embodiments, each complex of the first plurality of complexes hybridized to a labeling oligonucleotide further hybridizes to a complementary sequence on a capture polymer through the capture sequence on the labeling oligonucleotide. In some embodiments, the capture polymer is immobilized on a solid substrate.

b. One or More Additional Pluralities

Provided herein are compositions and methods which are or contain one or more additional pluralities of complexes. In some embodiments, each complex of the one or more pluralities of complexes is or contains a glycan binding agent. In some embodiments, each complex of the one or more pluralities of complexes is or contains a glycan-binding agent conjugated to a universal anchor. In some embodiments, each complex of the one or more pluralities of complexes is or contains a glycan-binding agent conjugated to a labeling oligonucleotide.

In some embodiments, each plurality of the one or more pluralities of complexes contains complexes with substantially identical glycan-binding agents. In some embodiments, each plurality of the one or more pluralities of complexes contains complexes with distinct glycan-binding agent. In some embodiments, each plurality of the one or more pluralities of complexes contains complexes with substantially identical glycan-binding agents conjugated to a universal anchor. In some embodiments, each plurality of the one or more pluralities of complexes contains complexes with a distinct glycan-binding agent conjugated to a universal anchor. In some embodiments, each plurality of the one or more pluralities of complexes contains complexes with substantially identical glycan-binding agents conjugated to a labeling oligonucleotide. In some embodiments, each plurality of the one or more pluralities of complexes contains complexes with a distinct glycan-binding agent conjugated to a labeling oligonucleotide. In some embodiments, the glycan-binding agent from each of the one or more additional pluralities is substantially identical to a) the glycan-binding agent of the first plurality, and b) the glycan-binding agent of the one or more additional pluralities. In some embodiments, the glycan-binding agent from each of the one or more additional pluralities is substantially distinct from a) the glycan-binding agent of the first plurality, and b) any other glycan-binding agent of the one or more additional pluralities.

In some embodiments, the glycan-binding agent of the one or more additional pluralities of complexes is selected from concanavalin A (ConA), a wheat germ agglutinin (WGA), a pea lectin, a soybean lectin, a lentil lectin, a ricin, a bacterial toxin, an influenza virus hemagglutinin, a C-type lectin, an S-type lectin, a P-type lectin, or an I-type lectin. In some embodiments, glycan-binding agent of the first plurality is concanavalin A (ConA). In some embodiments, glycan-binding agent of the first plurality is wheat-germ agglutinin (WGA).

In some embodiments, each plurality of the one or more additional pluralities of complexes is or contains 2 or more glycan-binding agents conjugated to a universal anchor. In some embodiments, each plurality of the one or more additional pluralities of complexes is or contains between 1 to 100 cell glycan-binding agents conjugated to a universal anchor. In some embodiments, each plurality of the one or more additional pluralities of complexes is or contains 100 or more glycan-binding agents conjugated to a universal anchor. In some embodiments, each plurality of the one or more additional pluralities of complexes is or contains 1000 or more glycan-binding agents conjugated to a universal anchor.

In some of any embodiments, each plurality of the one or more additional pluralities of complexes contains a substantially identical glycan-binding agent conjugated to a universal anchor, which is hybridized to a labeling oligonucleotide. In some of any embodiments, each plurality of the one or more additional pluralities of complexes contains a substantially identical glycan-binding agent conjugated to a universal anchor, which is hybridized to a labeling oligonucleotide which contains a binding site for a primer, a sample barcode sequence and a capture sequence. In some of any embodiments, each plurality of the one or more additional pluralities of complexes contains a substantially identical glycan-binding agent conjugated to a universal anchor, which is hybridized to a labeling oligonucleotide which contains a binding site for a primer, a sample barcode sequence, a capture sequence and a detectable label.

In some of any embodiments, each plurality of the one or more additional pluralities of complexes contains a substantially identical glycan-binding agent conjugated to a universal anchor, which is hybridized to a complementary sequence on a labeling oligonucleotide. In some of any embodiments, each plurality of the one or more additional pluralities of complexes contains a substantially identical glycan-binding agent conjugated to a universal anchor, which is hybridized to a complementary sequence on a binding site for a primer on a labeling oligonucleotide.

In some embodiments, the one or more additional pluralities comprises 1 plurality of complexes. In some embodiments, the one or more additional pluralities comprises between 1 to 100 pluralities of complexes. In some embodiments, the one or more additional pluralities comprises 10 or more pluralities of complexes. In some embodiments, the one or more additional pluralities comprises 20 or more pluralities of complexes. In some embodiments, the one or more additional pluralities comprises 30 or more pluralities of complexes. In some embodiments, the one or more additional pluralities comprises 40 or more pluralities of complexes. In some embodiments, the one or more additional pluralities comprises 50 or more pluralities of complexes. In some embodiments, the one or more additional pluralities comprises 100 or more pluralities of complexes. In some embodiments, the one or more additional pluralities comprises 1000 or more pluralities of complexes.

In some of any embodiments, each plurality of the one or more additional pluralities of complexes contains a distinct glycan-binding agent conjugated to a universal anchor, which is hybridized to a labeling oligonucleotide. In some of any embodiments, each plurality of the one or more additional pluralities of complexes contains a distinct glycan-binding agent conjugated to a universal anchor, which is hybridized to a labeling oligonucleotide which contains a binding site for a primer, a sample barcode sequence and a capture sequence. In some of any embodiments, each plurality of the one or more additional pluralities of complexes contains a distinct glycan-binding agent conjugated to a universal anchor, which is hybridized to a labeling oligonucleotide which contains a binding site for a primer, a sample barcode sequence, a capture sequence and a detectable label. In some of any embodiments, each plurality of the one or more additional pluralities of complexes contains a distinct glycan-binding agent conjugated to a universal anchor, which is hybridized to a complementary sequence on a labeling oligonucleotide. In some of any embodiments, t each plurality of the one or more additional pluralities of complexes contains a distinct glycan-binding agent conjugated to a universal anchor, which is hybridized to a complementary sequence on a binding site for a primer on a labeling oligonucleotide.

In some embodiments, each plurality of the one or more additional pluralities of complexes is hybridized to a labeling oligonucleotide which contains a substantially identical binding site for a primer. In some embodiments, each plurality of the one or more additional pluralities of complexes is hybridized to a labeling oligonucleotide which contains a distinct binding site for a primer. In some embodiments, each plurality of the one or more additional pluralities of complexes is hybridized to a labeling oligonucleotide which contains a substantially identical sample barcode sequence. In some embodiments, each plurality of the one or more additional pluralities of complexes is hybridized to a labeling oligonucleotide which contains a distinct sample barcode sequence. In some embodiments, each plurality of the one or more additional pluralities of complexes is hybridized to a labeling oligonucleotide which contains a substantially identical capture sequence. In some embodiments, each plurality of the one or more additional pluralities of complexes is hybridized to a labeling oligonucleotide which contains a distinct sample capture sequence.

In some embodiments, each sample barcode sequence within each plurality of the one or more additional pluralities comprises a substantially identical binding site for a primer. In some embodiments, each sample barcode sequence within each plurality of the one or more additional pluralities comprises a different binding site for a primer. In some embodiments, each sample barcode sequence within each plurality of the one or more additional pluralities comprises a substantially identical barcode sequence. In some embodiments, each sample barcode sequence within each plurality of the one or more additional pluralities comprises a different barcode sequence. In some embodiments, each sample barcode sequence within each plurality of the one or more additional pluralities comprises a substantially identical capture sequence. In some embodiments, each sample barcode sequence within each plurality of the one or more additional pluralities comprises a different capture sequence.

In some embodiments, the glycan-binding agent of the first plurality and the glycan binding agent of the one or more additional pluralities comprise a substantially identical glycan binding agent. In some embodiments, the glycan-binding agent of the first plurality and the glycan binding agent of the one or more additional pluralities comprise a distinct glycan binding agent.

In some embodiments, the sample barcode sequence within each of the one or more additional pluralities comprises a substantially identical barcode sequence, and the sample barcode sequence between the first plurality and the one or more additional pluralities comprises a distinct sample barcode sequence. In some embodiments, each complex of the one or more additional pluralities of complexes hybridized to a labeling oligonucleotide further hybridizes to a complementary sequence on a capture polymer. In some embodiments, each complex of the one or more additional pluralities of complexes hybridized to a labeling oligonucleotide further hybridizes to a complementary sequence on a capture polymer through the capture sequence on the labeling oligonucleotide. In some embodiments, the capture polymer is immobilized on a solid substrate.

### IV. Methods

The present disclosure provides methods and systems for enhancing multiplexing of samples. In some embodiments, the present disclosure provides methods and systems for increasing throughput in the analysis of samples. In some of any embodiments, any of the compositions described herein can be used in a single workflow which includes the processing, identification, and/or additional analysis of one or more samples and/or cell types.

In some of any embodiments of the compositions and methods described herein, a first or one or more additional pluralities of complexes can be used to bind to one or more cell features (i.e. glycans) which may be used to characterize a sample. In some embodiments, the first or one or more additional pluralities of complexes can be used to bind to a substantially identical cell feature (i.e. glycans) which may be used to characterize a homogenous or heterogenous pool of samples. In some embodiments, the first or one or more additional pluralities of complexes can be used to bind to a distinct or different cell feature (i.e. glycans) which may be used to characterize a homogenous or heterogenous pool of samples. In some embodiments, the first or one or more additional pluralities of complexes can be used to bind to one or more cell features (i.e. glycans) which may be used to characterize a homogenous or heterogenous pool of samples.

In some of any embodiments, the first or one or more additional pluralities of complexes are hybridized or bound to a labeling oligonucleotide that is indicative of the cell surface feature to which the complexes bind. For a description of example methods of use, see, e.g., U.S. Pat. No. 10,550,429; U.S. Pat. Pub. 20190177800; and U.S. Pat. Pub. 20190367969, each of which is herein entirely incorporated by reference for all purposes. Characterization of samples within a well may be performed. Such characterization can include, in non-limiting examples, imaging of the sample (e.g., cell or sub-cellular or cellular components) or derivatives thereof. Characterization techniques such as microscopy or imaging may be useful in measuring sample profiles in fixed spatial locations. For instance, when cells (e.g., fixed cells or un-fixed cells) are partitioned, optionally with beads, imaging of each microwell and the contents contained therein may provide useful information on cell doublet formation (e.g., frequency, spatial locations, etc.), cell-bead pair efficiency, cell viability, cell size, cell morphology, expression level of a biomarker (e.g., a surface marker, a fluorescently labeled molecule therein, etc.), cell or bead loading rate, number of cell-bead pairs, cell-cell interactions (when two or more cells are co-partitioned). Alternatively, or in addition to, imaging may be used to characterize a quantity of amplification products in the well.

In some embodiments, any of the compositions described herein can be used in methods to purify cells. In some embodiments, any of the compositions described herein can be used in cell separation methods. In some of any embodiments, any of the compositions provided herein can be used to isolate cells. In some of any embodiments, any of the compositions provided herein can be used in immunomagnetic cell separation, fluorescence-activated cell sorting (FACS), density gradient centrifugation, immunodensity cell isolation, microfluidic cell sorting, or any method known to one of skill in the art. Exemplary methods can be found in Amos et al. 2012 Cells Tissues Organs, Hu et al. 2016 Front. Cell Dev Biol, Almeida et al 2014 Pathobiology.

In some embodiments, any of the compositions described herein can be used in conjunction with a cell or tissue staining kits. Exemplary cell staining include live/dead staining, cell surface staining, Gram staining, Haematoxylin and Eosin staining, acid base fuchsin staining, Coomassie blue, DAPI, crystal violet, Eosin, Ethidium bromide, Hoechst, iodine, methylene blue, nile blue, rhodamide, safranin, malachite green, any fluorescent cell stain described herein in Section II C, or any kit known to one of skill in the art.

In some embodiments, any of the compositions described herein can be used in methods to determine cellular processes and disease states with high information content, including single-cell profiling. In some embodiments, any of the compositions or methods described herein can provide in-depth characterization of single cells by measurement of glycan expression levels and are compatible with single-cell sequencing systems. Among such known single cell sequencing platforms suitable for integration with the compositions and methods described herein is the CITE-Seq method, the Drop-seq method, including, but not limited to, microfluidic, plate-based, or microwell, Seq-Well™ method³⁵ and adaptations of the basic protocol, InDrop™ method² (1 Cell Bio) and HIVE™ scRNAseq. In another embodiment, a single cell sequencing platform suitable for integration with the compositions and methods described herein is 10× genomics single cell 3′ solution, or single cell V(D)J solution either run on Chromium controller, or dedicated Chromium single cell controller. Still other useful sequencing protocols for combination with the methods described herein include Wafergen iCell8™ method; Microwell-seq method, Fluidigm CI™ method and equivalent single cell products. Still other known sequencing protocols useful with the compositions and methods described herein include BD Resolve™ single cell analysis platform³⁷ (derived from Cyto-seq) and ddSeg⁶ (from Illumina® Bio-Rad® SureCell™ WTA 3′ Library Prep Kit for the ddSEQ™ System, 2017, Pub. No. 1070-2016-014-B, Illumina Inc., Bio-Rad Laboratories, Inc.). In still other embodiment, the compositions and methods described herein are useful with combinatorial indexing based approaches (sci-RNA-Seq™ method²⁰ or SPLiT-Seq™ method³⁰) and Spatial Transcriptomics, or comparable spatially resolved sequencing approaches. The methods and compositions described herein can also be used as an added layer of information on standard index sorting (FACS) and mRNA-sequencing-based approaches. In one embodiment, standard FACS panels are supplemented with glycan-binding agents detectable through plate-based sequencing. Still other sequencing protocols can be combined with the compositions and methods specifically described herein. In some of any embodiments, any of the methods described herein can be combined with RNA sequencing, DNA sequencing, protein detection and/or antibody based oligonucleotide protein detection.

In some embodiments, methods can employ detection protocols, including without limitation, PCR, Immuno-PCR and proximity ligation or proximity extension assay protocols, PEA, RCA, sequencing and fluorescence hybridization protocols and can be applied to operating platforms including Operetta (PerkinElmer). In some embodiments, any of the compositions or methods described herein can be used in diverse environments for detection of different targets, by employing any number of assays and methods for detection or targets in general. In one embodiment, a method for detecting one or more targets in a biological sample uses the compositions described herein.

In some of any embodiments, any of the methods and compositions described herein can be used, to determine the presence, amount, or absence of a sample, cell, target, ligand or oligonucleotide. In some embodiments, determining the amount of a sample, cell, target, ligand or oligonucleotide comprises determine an absolute, approximate, mean, average or relative amount of a sample, cell, target, ligand or oligonucleotide in a multiplex assay. Accordingly, in certain embodiments, methods and compositions described herein can be used to quantitate amounts of a sample, cell, target, ligand or oligonucleotide in a multiplex assay. In some embodiments, any of the compositions described herein can be used in a high-throughput method. In one embodiment, the compositions described herein are used in high-throughput protocols for detecting one or more glycans in a biological sample and can employ hundreds or thousands of wells containing the same or different samples.

In some of any embodiments, any of the methods and compositions described herein can be used, to determine the presence, amount, or absence of a subcellular or cellular component. In some embodiments, any of the methods or compositions described herein can be used to identify a subcellular component after isolation. In some of any embodiments, any of the methods or compositions described herein can be used to identify a nucleus, a ribosome, an endoplasmic reticulum, a Golgi apparatus, a cytoskeleton, a mitochondria, a vacuole and a vesicle. In some of any embodiments, any of the methods or compositions described herein can be used to identify a nucleus, a ribosome, an endoplasmic reticulum, a Golgi apparatus, a cytoskeleton, a mitochondria, a vacuole or a vesicle. In some embodiments, any of the methods or compositions described herein can be used to identify an isolated nucleus, ribosome, endoplasmic reticulum, Golgi apparatus, cytoskeleton, mitochondria, vacuole and vesicle. In some embodiments, any of the methods or compositions described herein can be used to identify an isolated nucleus, ribosome, endoplasmic reticulum, Golgi apparatus, cytoskeleton, mitochondria, vacuole or vesicle. In some embodiments, any of the methods or compositions described herein can be used to identify a non-isolated nucleus. In some embodiments, any of the methods or compositions described herein can be used to identify an non-isolated nucleus, ribosome, endoplasmic reticulum, Golgi apparatus, cytoskeleton, mitochondria, vacuole and vesicle. In some embodiments, any of the methods or compositions described herein can be used to identify an non-isolated nucleus, ribosome, endoplasmic reticulum, Golgi apparatus, cytoskeleton, mitochondria, vacuole or vesicle.

In some of any embodiments, any of the methods or compositions described herein can be used to identify a nucleus. In some embodiments, any of the methods or compositions described herein can be used to identify an isolated nucleus. In some embodiments, any of the methods or compositions described herein can be used to identify a non-isolated nucleus.

In some embodiments, any of the compositions or methods described herein include generating a first sample by contacting any of the first plurality of complexes described herein with a first plurality of target cells comprising one or more glycans, wherein the first plurality of complexes bind to the one or more glycans, generating one or more additional samples by contacting any of the one or more additional pluralities of complexes described herein with one or more additional pluralities of target cells each comprising one or more glycans, wherein the one or more additional pluralities of complexes bind to the one or more glycans, and pooling the first and the one or more additional samples. High-throughput protocols may also involve washing the biological sample to remove unbound complexes.

In some embodiments, any of the compositions or methods described herein include generating a first plurality of samples by contacting any of the first plurality of complexes described herein with a first plurality of target cells comprising one or more glycans, wherein the first plurality of complexes bind to the one or more glycans, generating one or more additional pluralities of samples by contacting any of the one or more additional pluralities of complexes described herein with one or more additional pluralities of target cells each comprising one or more glycans, wherein the one or more additional pluralities of complexes bind to the one or more glycans, pooling the first and the one or more additional pluralities of samples, partitioning each sample from the pool of first and one or more additional pluralities of samples into a plurality of partitions, wherein each partition of the plurality of partitions comprises a single cell associated with a complex, within the partition, hybridizing the capture sequence on the complex associated with a capture polymer on a solid substrate, obtaining sequence information from the pool of first and one or more additional pluralities of samples to identify the first and one or more additional pluralities of target cells from the pool generated.

In some embodiments, any of the compositions or methods described herein include identifying a first cell type and one or more additional cell types from a mixture of cells, which includes generating a first plurality of samples by contacting any of the first plurality of complexes described herein with a first plurality of target cells comprising one or more glycans, wherein the first plurality of complexes bind to the one or more glycans, generating one or more additional pluralities of samples by contacting any of the one or more additional pluralities of complexes described herein with one or more additional pluralities of target cells each comprising one or more glycans, wherein the one or more additional pluralities of complexes bind to the one or more glycans, pooling the first and the one or more additional pluralities of samples, partitioning each sample from the pool of first and one or more additional pluralities of samples into a plurality of partitions, wherein each partition of the plurality of partitions comprises a single cell associated with a complex, within the partition, hybridizing the capture sequence on the complex with a capture polymer on a solid substrate obtaining sequence information from the pool of first and one or more additional pluralities of samples to identify the first and one or more additional pluralities of target cells from the pool generated in (c).

In some of any embodiments, the method further includes identifying one or more additional pluralities of target cells based on both the glycan profiles and antibody profiles. In some of any embodiments, the method further includes wherein the generating one or more additional pluralities of samples further includes contacting the target cells with an antibody comprising a labeling oligonucleotide. In some of any embodiments, the method further includes contacting the target cells with an antibody comprising an oligonucleotide and or a polyethylene glycol (PEG) conjugated to a glycan present on the antibody prior to pooling. In some of any embodiments, the method further includes blocking the antibody with an unconjugated lectin.

In some of any embodiments, the method further includes lysing the cell after hybridizing the capture sequence to the capture polymer. Suitable lysis techniques can involve exposure of the cells to detergents, detergent-buffer solutions, such as RIPA buffer, IP-lysis buffers, M-PER or B-PER reagent solutions (Pierce Chemical) and the like. In some of any embodiments, the first cell type and the second cell type are selected from the group comprising human cells, non-human cells, healthy cells, cells associated with a disease or disorder, cancer cells, non-cancer cells, cells from the same subject, cells from a different subject, activated cells, non-activated cells, fresh cells, fixed cells, permeabilized cells, cells from a human subject or cells from a cell line.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

In some of any embodiments, the compositions described herein can be used in spatial analysis methodologies described in but not limited to U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodrigues et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018.

In some embodiments, any of the sample barcode sequences provided herein is a spatial barcode sequence. In some of any embodiments, the spatial barcode identifies one or more cells, and/or contents of the one or more cells, associated with a particular spatial location. In some of any embodiments, the method includes a spatially-barcoded array (e.g., including labeling oligonucleotides which contain a spatial sample barcode sequence (spatially-barcoded labeling oligonucleotides)). In some embodiments, the method includes the cleavage of spatially-barcoded labeling oligonucleotides from an array, which promote the spatially-barcoded labeling oligonucleotides towards and/or into the biological sample. In some embodiments, the method includes the cleavage of spatially-barcoded labeling oligonucleotides from an array, which promote the spatially-barcoded labeling oligonucleotides towards and/or onto the biological sample.

Spatial information can provide information of biological and/or medical importance. For example, the methods and compositions described herein can allow for identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder

In some of any embodiments, provided herein is a method of identifying a sample through sequential hybridization, which includes contacting any of the first plurality of complexes described herein each comprising a substantially identical glycan-binding agent conjugated to a universal anchor with a first plurality of target cells comprising one or more glycans, wherein the first plurality of complexes bind to the one or more glycans, reversibly hybridizing the first plurality of complexes to a complementary sequence on a first labeling oligonucleotide comprising a binding site for a primer, a sample barcode sequence, a capture sequence, and a detectable label, detecting the detectable label, de-hybridizing the first plurality of complexes, reversibly hybridizing the first plurality of complexes to a complementary sequence on a second labeling oligonucleotide comprising a binding site for a primer, a sample barcode sequence, a capture sequence, and a detectable label, detecting the detectable label and identifying the sample based on the detectable labels.

In some of any embodiments, provided herein is a method of identifying a sample through sequential hybridization, including contacting a first plurality of complexes each comprising a substantially identical glycan-binding agent conjugated to a universal anchor with a first plurality of target cells comprising one or more glycans, wherein the first plurality of complexes bind to the one or more glycans, contacting one or more additional pluralities of complexes, each plurality comprising a distinct glycan-binding agent conjugated to a universal anchor, with the first plurality of target cells comprising one or more glycans, wherein the one or more additional pluralities of complexes bind to the one or more glycans, incubating the first plurality of complexes and the one or more additional pluralities of complexes with a first labeling oligonucleotide comprising a binding site for a primer, a sample barcode sequence, a capture sequence, and a detectable label, under conditions to reversibly hybridize the first labeling oligonucleotide to the universal anchor, detecting the detectable label, de-hybridizing the first plurality of complexes and the one or more additional pluralities of complexes, incubating the first plurality of complexes and the one or more additional pluralities of complexes with a second labeling oligonucleotide comprising a binding site for a primer, a sample barcode sequence, a capture sequence, and a detectable label, under conditions to reversibly hybridize the second labeling oligonucleotide to the universal anchor, detecting the detectable label and identifying the sample based on the detectable labels.

In some of any embodiments, the sample barcode sequence on the second labeling oligonucleotide is different than the sample barcode sequence from the first labeling oligonucleotide. In some of any embodiments, the detectable label on the second labeling oligonucleotide is different than the detectable label from the first labeling oligonucleotide. Provided herein is a method of associating presence or abundance of a glycan with a location of a tissue sample, which includes delivering any of the first plurality of complexes described herein to a tissue sample affixed to a support, under conditions to specifically bind the first plurality of complexes to the glycan at a location of the tissue sample, sequencing all or a portion of the first plurality of complexes, and using the determined sequence to associate presence or abundance of the target biological molecule with the location of the tissue sample.

In some of any embodiments, any of the methods described herein further includes delivering any of the one or more additional pluralities of complexes described herein to a tissue sample affixed to a support, under conditions to specifically bind the one or more additional pluralities of complexes to the glycan at a location of the tissue sample, sequencing all or a portion of the one or more additional pluralities of complexes, and using the determined sequence to associate presence or abundance of the target biological molecule with the location of the tissue sample.

In some of any embodiments, the method further comprises profiling both glycans and antibodies simultaneously. In some of any embodiments, the method further includes identifying one or more additional pluralities of tissue samples based on both the glycan profiles and antibody profiles. In some of any embodiments, the method further includes wherein the generating one or more additional pluralities of samples further includes contacting the tissue samples with an antibody comprising a labeling oligonucleotide. In some of any embodiments, the method further includes contacting the tissue sample with an antibody comprising an oligonucleotide and or a polyethylene glycol (PEG) conjugated to a glycan present on the antibody prior to sequencing. In some of any embodiments, the method further includes blocking the antibody with an unconjugated lectin.

### V. Samples and Sample Preparation

Provided herein are samples or biological samples which are naturally-occurring samples or deliberately designed or synthesized samples. In some embodiments, the sample contains a population of cells or cell fragments, including without limitation cell membrane components, exosomes, and sub-cellular components. The cells may be a homogenous population of cells, such as isolated cells of a particular type, or a mixture of different cell types, such as from a biological fluid or tissue of a human or mammalian or other species subject. Still other samples for use in the methods and with the compositions include, without limitation, blood samples, including serum, plasma, whole blood, and peripheral blood, saliva, urine, vaginal or cervical secretions, amniotic fluid, placental fluid, cerebrospinal fluid, or serous fluids, mucosal secretions (e.g., buccal, vaginal or rectal), cell samples and tissue samples. Still other samples include a blood-derived or biopsy-derived biological sample of tissue or a cell lysate (i.e., a mixture derived from tissue and/or cells). Other suitable tissue includes hair, fingernails and the like. Still other samples include libraries of antibodies, antibody fragments and antibody mimetics like affibodies. Such samples may further be diluted with saline, buffer or a physiologically acceptable diluent. Alternatively, such samples are concentrated by conventional means. Still other samples can be synthesized or engineered collections of chemical molecules, proteins, antibodies or any other of the targets described herein. The compositions and kits described above can be used in diverse environments for detection of different targets, by employing any number of assays and methods for detection or targets in general.

The sample can be any biological sample, including for example, blood, tissue, cells, cell cultures, urine, or saliva. The biological sample may be derived from another sample. The sample may be a tissue sample, such as a biopsy, core biopsy, needle aspirate, or fine needle aspirate sample. The sample may be a fluid sample, such as a blood sample, urine sample, blister sample, or saliva sample. The sample may be a skin sample. The sample may be a cheek swab. The sample may be a plasma or serum sample. A sample can comprise of a fraction isolated from a bodily sample, which may be selected from the group consisting of blood, plasma, serum, urine, saliva, mucosal excretions, sputum, stool, and tears. Another example of a sample can be a tube of cells from a donor or subject, from a particular tissue at a particular point in time, and possibly enriched for particular cells. The terms donor and subject are used interchangeably herein. A donor or a subject is an individual from which samples are obtained. The donor or subject can be a mammalian or vertebrate subject, including for example, a human, swine, cow, camelid, lamprey, monkey, ape, dog, cat, mouse, or rat. The sample may be a cell or derivative of a cell (such as a cell nucleus). The sample may be a rare cell from a population of cells. The sample may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The sample may be a constituent of a cell. The sample may be or may include DNA, RNA, organelles, proteins, or any combination thereof. The sample may include or be processed to include a matrix (e.g., a gel or polymer matrix) comprising a cell or one or more constituents from a cell, such as DNA, RNA, organelles, proteins, or any combination thereof, from the cell. For a description of exemplary gel or polymer matrix embedded samples, see, e.g., U.S. Pat. Nos. 10,584,381; 10,428,326; and U.S. Pat. Pub. 20190100632, each of which are incorporated herein by reference in their entireties. The sample may be obtained from a tissue of a subject. The sample may be a hardened cell. Such hardened cell may or may not include a cell wall or cell membrane. In some instances, the sample may include one or more constituents of a cell, but may not include other constituents of the cell. An example of such constituents is a nucleus or an organelle. A cell may be a live cell. The live cell may be capable of being cultured, for example, being cultured when enclosed in a gel or polymer matrix or cultured when comprising a gel or polymer matrix.

A variety of steps can be performed to prepare a biological sample for analysis. Except where indicated otherwise, the preparative steps described below can generally be combined in any manner to appropriately prepare a particular sample for analysis. A biological sample can be harvested from a subject (e.g., via surgical biopsy, whole subject sectioning), grown in vitro on a growth substrate or culture dish as a population of cells, or prepared as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material. In some embodiments, the biological sample (e.g., a tissue section as described above) can be prepared by deep freezing at a temperature suitable to maintain or preserve the integrity (e.g., the physical characteristics) of the tissue structure. Such a temperature can be, e.g., less than −20° C., or less than −25° C., −30° C., −40° C., −50° C., −60° C., −70° C., −80° C. −90° C., −100° C., −110° C., −120° C., −130° C., −140° C., −150° C., −160° C., −170° C., −180° C., −190° C., or −200° C. The frozen tissue sample can be sectioned, e.g., thinly sliced, onto a substrate surface using any number of suitable methods. In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes). As an alternative to formalin fixation described above, a biological sample can be fixed in any of a variety of other fixatives to preserve the biological structure of the sample prior to analysis. For example, a sample can be fixed via immersion in ethanol, methanol, acetone, formaldehyde (e.g., 2% formaldehyde), paraformaldehyde-Triton, glutaraldehyde, or combinations thereof.

As an alternative to paraffin embedding described above, a biological sample can be embedded in any of a variety of other embedding materials to provide a substrate to the sample prior to sectioning and other handling steps. In general, the embedding material is removed prior to analysis of tissue sections obtained from the sample. Suitable embedding materials include, but are not limited to, waxes, resins (e.g., methacrylate resins), epoxies, and agar. To facilitate visualization, biological samples can be stained using a wide variety of stains and staining techniques. In some embodiments, a sample can be stained using any number of biological stains, including but not limited to, acridine orange, Bismarck brown, carmine, Coomassie blue, Cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus. A “hydrogel” as described herein can include a cross-linked 3D network of hydrophilic polymer chains. A “hydrogel subunit” can be a hydrophilic monomer, a molecular precursor, or a polymer that can be polymerized (e.g., cross-linked) to form a three-dimensional (3D) hydrogel network.

In the case of encapsulated biological particles (e.g., cells), a biological particle may be included in a droplet that contains lysis reagents in order to release the contents (e.g., contents containing one or more analytes (e.g., bioanalytes)) of the biological particles within the droplet. In such cases, the lysis agents can be contacted with the biological particle suspension concurrently with, or immediately prior to the introduction of the biological particles into the droplet or particle formation region, for example, through an additional channel or channels upstream or proximal to a second channel or a third channel that is upstream or proximal to a second droplet or particle formation region. Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, Mo.), as well as other commercially available lysis enzymes. Other lysis agents may additionally, or alternatively, be contained in a droplet with the biological particles (e.g., cells) to cause the release of the biological particles' contents into the droplets or particles. For example, in some cases, surfactant based lysis solutions may be used to lyse cells, although these may be less desirable for emulsion based systems where the surfactants can interfere with stable emulsions. In some cases, lysis solutions may include non-ionic surfactants such as, for example, TRITON X-100 and TWEEN 20. In some cases, lysis solutions may include ionic surfactants such as, for example, sarcosyl and sodium dodecyl sulfate (SDS). In some embodiments, lysis solutions are hypotonic, thereby lysing cells by osmotic shock. Electroporation, thermal, acoustic or mechanical cellular disruption may also be used in certain cases, e.g., non-emulsion based droplet formation such as encapsulation of biological particles that may be in addition to or in place of droplet formation, where any pore size of the encapsulate is sufficiently small to retain nucleic acid fragments of a desired size, following cellular disruption.

In addition to the lysis agents, other reagents can also be included in droplets with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated biological particles (e.g., cells), the biological particles may be exposed to an appropriate stimulus to release the biological particles or their contents from a microcapsule within a droplet. For example, in some cases, a chemical stimulus may be included in a droplet along with an encapsulated biological particle to allow for degradation of the encapsulating matrix and release of the cell or its contents into the larger droplet.

### VI. Kits and Compositions

Provided herein are kits comprising any of the compositions described herein. Also provided herein are kits that include parts and instructions for performing the methods described herein. In some embodiments, provided herein are kits comprising any of the first pluralities of complexes provided herein. In some embodiments, provided herein are kits comprising any of the one or more additional pluralities of complexes provided herein. In some embodiments, first plurality of complexes and the one or more additional pluralities of complexes are packaged separately. In some of any embodiments, any of the kits provided herein further contain a plurality of labeling oligonucleotides. In some of any embodiments, any of the kits provided herein further contain any of the labeling oligonucleotides described herein. In some embodiments, the labeling oligonucleotides are packaged separately.

Provided herein is a kit which contains a plurality of substantially identical complexes comprising a glycan-binding agent and a universal anchor; a first plurality of labeling oligonucleotides comprising a binding site for a primer, a substantially identical sample barcode sequence and a capture sequence; and one or more additional pluralities of labeling oligonucleotides, wherein each labeling oligonucleotide comprises a binding site for a primer, a substantially identical sample barcode sequence and a capture sequence; wherein the sample barcode sequence between each of the first and one or more additional pluralities of labeling oligonucleotides comprises a distinct sample barcode sequence from the first plurality of labeling oligonucleotides or any other additional pluralities of labeling oligonucleotides.

Provided herein is a kit which contains a first plurality of complexes comprising substantially identical glycan-binding agents conjugated to a universal anchor; one or more additional pluralities of complexes, each plurality comprising a substantially identical glycan-binding agent conjugated to a universal anchor; wherein the glycan binding agent between the first plurality and the one or more additional pluralities comprises a distinct glycan binding agent; a first plurality of labeling oligonucleotides comprising a binding site for a primer, a substantially identical sample barcode sequence and a capture sequence; and one or more additional pluralities of labeling oligonucleotides comprising a binding site for a primer, a substantially identical sample barcode sequence and a capture sequence; wherein the sample barcode sequence between each of the first and one or more additional pluralities of labeling oligonucleotides comprises a distinct sample barcode sequence. In some embodiments, each component is packaged separately.

The kit can also include reagents for carrying out the methods disclosed herein. For example, in some instances, the kit includes reagents for carrying out reverse transcription reactions (e.g., a reverse transcriptase); nucleic acid quantification as described herein; addition of fragments to nucleic acid sequences; and nucleic acid clean up. In some instances, the kit includes reagents necessary to fix, stain, and de-stain the biological sample.

In some instances, the kit includes components that allow for multiple reactions. In some instances, the kit includes components that allow for target hybridization. In some instances, the components for target hybridization include one or more of universal blockers, hybridization buffer, hybridization enhancer, equilibrium buffer (concentrated or 1×), wash buffer (concentrated or 1×), primers, control samples, or any combination thereof. In some instances, the kit includes components for amplifying the library of nucleic acids. In some instances, the components for amplifying a library of nucleic acids includes a mixture that aids in amplification, primers, or any combination thereof.

In some instances, the kit further includes instructions for carrying out library preparation and nucleic acid detection using the methods described herein. The various components of the kit may be present in separate containers or certain compatible components may be pre-combined into a single container. In some embodiments, the kits further contain instructions for using the components of the kit to practice the provided methods. In some embodiments, the kits can contain reagents and/or consumables required for performing one or more steps of the provided methods. In some embodiments, the kits contain reagents for embedding the biological sample. In some embodiments, the kits contain reagents, such as enzymes and buffers for ligation and/or amplification, such as ligases and/or polymerases. In some aspects, the kit can also include any of the reagents described herein, e.g., wash buffer, and ligation buffer. In some embodiments, the kits contain reagents for detection and/or sequencing, such as barcode detection probes or detectable labels. In some embodiments, the kits optionally contain other components, for example: nucleic acid primers, enzymes and reagents, buffers, nucleotides, modified nucleotides, reagents for additional assays.

### VII. Definitions

Unless otherwise defined, all technical and scientific terms used herein have the same meaning as commonly understood by one of ordinary skill in the art. Preferred methods and compositions are described herein, although methods and compositions similar or equivalent to those described herein can be used in practice or testing of the present technology. All publications, patent applications, patents and other references mentioned herein are incorporated by reference in their entirety. The methods, compositions, and examples disclosed herein are illustrative only and not intended to be limiting.

The term “polymer” generally refers to any backbone of multiple monomeric components that can function to bind to the selected ligand and/or anchor component and be utilized in a downstream assay. This assay may utilize the activity of one or more enzymes, for example reverse transcriptases, DNA or RNA polymerases, DNA or RNA ligases, etc. Such polymers or monomeric components include oligonucleotides (e.g., DNA, RNA, synthetic or recombinant DNA or RNA bases or analogs of DNA or RNA bases), peptide nucleic acids (i.e., a synthetic nucleic acid analog in which natural nucleotide bases are linked to a peptide-like backbone instead of the sugar-phosphate backbone found in DNA and RNA), locked nucleic acids (LNA; see, e.g., Grunweller A and Hartmann R K, “Locked nucleic acid oligonucleotides: the next generation of antisense agents?”, BioDrugs 2007. 21(4):235-43)), or polyamide polymers (see, e.g. Dervan P B and Burli, R W, Sequence-specific DNA recognition by polyamides”, Curr. Opn Chem. Biol. 1999, 3:688-693). However, wherever the term “oligonucleotide”, “nucleic acid” or nucleotide” or a similar specific example of a monomer or polymer is used in this specification, it should also be understood to mean that the composition or complex may be formed of any suitable polymer as described in this paragraph.

The terms “first plurality” and “additional pluralities” are used throughout this specification generally as reference terms to distinguish between various forms and components of complexes. “First plurality” generally refers to complexes with a glycan binding agent and an oligonucleotide comprising an anchor, a sample barcode sequence and a binding site for a primer. The term “additional pluralities” generally refers to complexes that differ from any other complex used in the compositions and methods defined herein in the identity of the sample barcode sequence or the glycan binding agent.

The term “universal” generally refers to a sequence that is substantially identical to other sequences in a composition. In some embodiments, the term “universal” is used to define a “universal anchor”. In some embodiments, the “universal anchor” is an anchor that contains the same sequence or a substantially identical sequence to all other anchors in a complex or composition. In some embodiments, the term “universal” is used to define a “universal primer”. In some embodiments, the “universal primer” is a primer that contains the same or a substantially identical sequence to all other primers in a complex or composition.

The term “substantially identical” generally refers to two or more components that contain the same structure or sequence. In some embodiments, two or more components are “substantially identical” if they contain 90%, 91%, 92%, 93%, 94%, 95%, 96%, 97%, 98% 99% or 100% similarity in structure or sequence. In some embodiments, “substantially identical” generally refers to a plurality of complexes or components, which contain the same basic structure. In some embodiments, the basic structure refers the same glycan-binding agent. In some embodiments, each one of the substantially identical plurality of complexes shares the same target and/or ligand. In some embodiments, a “substantially identical” sequence refers to an anchor sequence. In some embodiments, a “substantially identical” sequence refers to a sample barcode sequence.

The term “substantially complementary” generally refers to two or more components that contain 90%, 91%, 92%, 93%, 94%, 95%, 96%, 97%, 98% 99% or 100% similarity in a structure or sequence that complement a respective structure or sequence.

The term “attachment” or “attach” or “conjugate” generally describes the interaction between the components of the pluralities of complexes is meant covalent attachments or a variety of non-covalent types of attachment. Other attachment chemistries useful in assembling the pluralities of complexes described herein include, but are not limited to, thiol-maleimide, thiol-haloacetate, amine-NHS, amine-isothiocyanate, azide-alkyne (CuAAC), tetrazole-cyclooctene (iEDDA).

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another. In some embodiments, the hybridization is nucleic acid hybridization. Nucleic acid hybridization involves providing a probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (for example, low temperature and/or high salt) hybrid duplexes (for example, DNA:DNA, RNA:RNA, or RNA:DNA) will form even where the annealed sequences are not perfectly complementary. Thus, specificity of hybridization is reduced at lower stringency. Conversely, at higher stringency (for example, higher temperature or lower salt) successful hybridization requires fewer mismatches. In some embodiments, the hybridized nucleic acids are detected by detecting one or more labels attached to the sample nucleic acids.

The term “ligand” generally refers to any naturally occurring or synthetic biological or chemical molecule which is used to bind specifically to a single identified target. The binding can be covalently or non-covalent, i.e., conjugated or by any known means taking into account the nature of the ligand and its respective target. The terms “first ligand” and “additional ligand” refer to ligands that bind to different targets or different portions of a target. For example, multiple “first ligands” bind to the same target at the same site. Multiple additional ligands bind to a target different than the first ligand and different than any additional ligand. The first and additional ligands, may be independently selected from a glycan, a lectin. In some embodiments, the ligand(s) of the complexes can also be directly labeled with one or more detectable labels, such as fluorophores (see labels discussed below) that can be measured by methods independent of the methods of measuring or detecting any of the complexes described otherwise herein.

The term “detectable label” generally refers to a reagent, moiety or compound capable of providing a detectable signal, depending upon the assay format employed. A label may be associated with the complex as a whole, or with the ligand only, or with the oligonucleotide or a functional portion thereof. Alternatively, different labels may be used for each component of the complex. Such labels are capable, alone or in concert with other compositions or compounds, of providing a detectable signal. In some embodiments, the labels are desirably interactive to produce a detectable signal. Most desirably, the label is detectable visually, e.g. colorimetrically.

The term “antibody” generally refers to a monoclonal antibody, a synthetic antibody, a recombinant antibody, a chimeric antibody, a humanized antibody, a human antibody, a CDR-grafted antibody, a multispecific binding construct that can bind two or more targets, a dual specific antibody, a bi-specific antibody or a multi-specific antibody, or an affinity matured antibody, a single antibody chain or an scFv fragment, a diabody, a single chain comprising complementary scFvs (tandem scFvs) or bispecific tandem scFvs, an Fv construct, a disulfide-linked Fv, a Fab construct, a Fab′ construct, a F(ab′)2 construct, an Fc construct, a monovalent or bivalent construct from which domains non-essential to monoclonal antibody function have been removed, a single-chain molecule containing one V_{L}, one V_{H} antigen-binding domain, and one or two constant “effector” domains optionally connected by linker domains, a univalent antibody lacking a hinge region, a single domain antibody, a dual variable domain immunoglobulin (DVD-Ig) binding protein or a nanobody. Also included in this definition are antibody mimetics such as affibodies, i.e., a class of engineered affinity proteins, generally small (˜6.5 kDa) single domain proteins that can be isolated for high affinity and specificity to any given protein target.

The term “oligonucleotide sequence” generally refers to the portion of the complex which is associated with the target or ligand. As stated above, this association can be covalent, non-covalent or by any suitable conjugation and employing any suitable linker. The oligonucleotide sequence is formed by a series of functional polymeric elements, e.g., nucleic acid sequences or other polymers as defined above, each having a function as defined herein. The ligand can be attached to the oligonucleotide sequence at its 5′ end or at any other portion, provided that the attachment or conjugation does not prevent the functions of the components of any of the oligonucleotide sequences provided herein. In general, the oligonucleotide sequence can be any length that accommodates the lengths of its functional components. In some embodiments, the oligonucleotide sequence is between 20 and 100 monomeric components, e.g., nucleic acid bases, in length. In some embodiments, the oligonucleotide sequence is at least 20, 30, 40, 50, 60, 70, 80, 90 or over 100 monomeric components, e.g., nucleic acid bases, in length. In other embodiments, the oligonucleotide sequence is 200 to about 400 monomeric components, e.g., nucleotides, in length. In some embodiments, any of the compositions or complexes provided herein are made up of deoxyribonucleic acids (DNA). In some embodiments, the oligonucleotide sequence is a DNA sequence. In other embodiments, the oligonucleotide sequence, or portions thereof, comprises modified DNA bases. Modification of DNA bases are known in the art, and can include chemically modified bases including labels. In some embodiments, the oligonucleotide sequence, or portions thereof, comprises ribonucleic acid (RNA) sequences or modified ribonucleotide bases. Modification of RNA bases are known in the art, and can include chemically modified bases including labels. In still other embodiments, different portions of the oligonucleotide sequence can comprise DNA and RNA, modified bases, or modified polymer connections (including but not limited to PNAs and LNAs). For a description of modifications to oligonucleotides, see commercial suppliers, e.g., Integrated DNA Technologies, USA website; Custom Oligonucleotide Modifications Guide, Sigma-Aldrich, World Wide Web Uniform Resource Locator: sigmaaldrich.com/technical-documents/articles/biology/custom-dna-oligos-modifications.html, and Modified Oligonucleotides, TriLink, World Wide Web Uniform Resource Locator: trilinkbiotech.com/oligo/modifiedoligos.asp. As described above, in still other embodiments, any of the compositions or complexes provided herein is composed of polyamides, PNA, etc.

The term “unique molecular identifier” (UMI), also called equivalently a “Random Molecular Tag” (RMT), generally refers to a random sequence of monomeric components of a polymer, e.g., nucleotide bases, which can be a functional element of any of the compositions described herein. The UMI permits identification of amplification duplicates of any of the oligonucleotide sequences with which it is associated. In some embodiments, one or more UMI may be associated with a single oligonucleotide sequence. The UMI may be positioned 5′ or 3′ to the sample barcode. In some embodiments, the UMI may be inserted into any position or oligonucleotide sequence as part of the described methods. In some embodiments of the methods described herein, depending on which RNA-sequencing method is used, a UMI is added during the method. However, not all RNA-seq methods make use of UMIs. In some examples of single cell droplet RNA-sequencing, another UMI is introduced during reverse transcription. Each UMI is specific for its oligonucleotide sequence. In some embodiments, each complex within any of the compositions described herein differs only in the sequence of its UMI. Each additional complex may also have its own UMI, which is not present on duplicate complexes that differ from each other in target, sample barcode, capture sequence and universal anchor specificity. Similarly, as used in the various methods described herein, a UMI may be associated with a polynucleotide sequence that is immobilized on a substrate. Each UMI may be different from any other UMI used in the compositions or methods. In some embodiments, the UMI is formed of a random sequence of DNA, RNA, modified bases or combinations of these bases or other monomers of polymers. In some embodiments, a UMI is about 8 monomeric components, e.g., nucleotides, in length. In other embodiments, each UMI can be at least about 1 to 100 monomeric components, e.g., nucleotides, in length. Thus in various embodiments, the UMI is formed of a random sequence of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99 or up to 100 monomeric components, e.g., nucleic acids.

The term “linker” generally refers to any moiety used to attach or associate the ligand to any of the compositions or complexes provided herein and/or to the oligonucleotide sequence portion of any of the compositions or complexes provided herein. Thus, in some embodiments, the linker is a covalent bond. In other embodiments, the linker is a non-covalent bond. In some embodiments, the linker is composed of at least one to about 25 atoms. Thus, in various embodiments, the linker is formed of at least 2, 3, 4, 5, 6, 7, 8, 9, 10, 11, 12, 13, 14, 15, 16, 17, 18, 19, 20, 21, 22, 23, 24 or 25 atoms. In still other embodiments, the linker is at least one to about 60 nucleic acids. Thus in various embodiments, the linker is formed of a sequence of at least 2,3,4,5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, up to 60 nucleic acids. In yet other embodiments, the linker refers to at least one to about 30 amino acids. Thus, in various embodiments, the linker is formed of a sequence of at least 2, 3, 4, 5, 6, 7, 8, 9, 10, 11, 12, 13, 14, 15, 16, 17, 18, 19, 20, 21, 22, 23, 24, 25, 26, 27, 28, 29, up to about 30 amino acids. In still other embodiments, the linker can be a larger compound or two or more compounds that associate covalently or non-covalently. In still other embodiments, the linker can be a combination of the linkers defined herein. The linkers used in the complexes, compositions and methods are in some embodiments cleavable. The linkers used in the complexes, compositions and methods are in some embodiments non-cleavable. Without limitation, in some embodiments, the linker is a disulfide bond. In some examples, the linker comprises a complex of biotin bound to any of the oligonucleotide sequences provided herein by a disulfide bond, with streptavidin fused to the ligand. In some embodiments, the biotin is bound to the ligand and the streptavidin is fused to any of the oligonucleotide sequences provided herein. Although examples show the linker bound to the 5′ end of the oligonucleotide of any of the compositions or complexes herein, in some embodiments, the linker may be covalently attached or conjugated other than covalently to any oligonucleotide sequence portion of compositions or complexes herein. In yet other embodiments, when the ligand is a recombinant or synthesized glycan, the linker can be engineered into the glycan sequence to facilitate 1:1 coupling to any of the complexes or compositions provided herein, thereby simplifying manufacturing of the ligand of any of the complexes or compositions. For example, a Halotag® linker can be engineered into the selected ligand (e.g., antibody) or into any of the complexes or compositions, for such purposes. Additionally, or alternatively, the ligand is linked to any of the complexes or compositions upon production in the same cell. See, e.g., the Halotag® protocols described by Flexi® Vector Systems Technical Manual (TM254-revised 5/17), copyright 2017 by Promega Corporation; and Janssen D. B., Evolving haloalkaline dehalogenase, Curr. Opin. Chem. Biol., 2004, 8:150-159.

The term “immobilized” generally means that the capture polymer is attached to a solid substrate resulting in reduction or loss of mobility via physical adsorption through charge-charge interaction or hydrophobic interaction, covalent bonding, Streptavidin-Biotin interaction or affinity coupling.

The term “substrate” generally means a microparticle (bead), a microfluidics microparticle (bead), a slide, a multi-well plate, a microwell, a nanowell, or a chip. The substrates are conventional and can be glass, plastic or of any conventional materials suitable for the particular assay or diagnostic protocols.

The term “phosphorothioate (PS) bond” substitutes a sulfur atom for a non-bridging oxygen in the phosphate backbone of an oligo. This modification renders the internucleotide linkage resistant to nuclease degradation. A “phosphorothioate (PS) bond” is indicated with a * in Example 1 and refers to a phosphorothioate (PS) bond between the two nucleotide residues.

For simplicity and ease of understanding, throughout this specification, certain specific examples are provided to teach the construction, use and operation of the various elements of the compositions and methods described herein. Such specific examples are not intended to limit the scope of this description.

### EXEMPLARY EMBODIMENTS

1. A composition for sample identification, comprising:
   a first plurality of complexes each comprising a substantially identical glycan-binding agent conjugated to a universal anchor.
2. The composition of embodiment 1, wherein the universal anchor of the first plurality comprises an amine-modified oligonucleotide sequence.
3. The composition of embodiment 1, wherein the universal anchor of the first plurality comprises a locked nucleic acid (LNA).
4. The composition of any of embodiments 1-3, wherein the universal anchor of the first plurality comprises at least 3 or more nucleotide bases which hybridize to a complementary sequence on a labeling oligonucleotide.
5. The composition of embodiment 4, wherein the labeling oligonucleotide further comprises a binding site for a primer, a sample barcode sequence, and a capture sequence.
6. The composition of embodiment 5, wherein the binding site for the primer comprises the complementary sequence on the labeling oligonucleotide.
7. The composition of embodiment 5 or embodiment 6, wherein each sample barcode sequence within the first plurality comprises a substantially identical barcode sequence.
8. The composition of any of embodiments 1-7, further comprising one or more additional pluralities of complexes, wherein each complex within the one or more additional pluralities comprises a substantially identical glycan-binding agent conjugated to a universal anchor.
9. The composition of embodiment 8, wherein the glycan-binding agent from each of the one or more additional pluralities is substantially identical to a) the glycan-binding agent of the first plurality, and b) the glycan-binding agent of the one or more additional pluralities.
10. The composition of embodiment 8, wherein the glycan-binding agent from each of the one or more additional pluralities is substantially distinct from a) the glycan-binding agent of the first plurality, and b) any other glycan-binding agent of the one or more additional pluralities.
11. The composition of any of embodiments 8-10, wherein the universal anchor from the one or more additional pluralities comprises an amine-modified oligonucleotide sequence.
12. The composition of any of embodiments 8-10, wherein the universal anchor from the one or more additional pluralities of complexes comprises a locked nucleic acid (LNA).
13. The composition of any of embodiments 8-12, wherein the universal anchor of the one or more additional pluralities comprises at least 3 or more nucleotide bases which hybridize to a complementary sequence on a labeling oligonucleotide.
14. The composition of embodiment 13, wherein the labeling oligonucleotide comprises a binding site for a primer, a sample barcode sequence, and a capture sequence.
15. The composition of embodiment 14, wherein the binding site for the primer comprises the complementary sequence on the labeling oligonucleotide.
16. The composition of embodiment 14 or embodiment 15, wherein the sample barcode sequence within each of the one or more additional pluralities comprises a substantially identical barcode sequence, and wherein the sample barcode sequence between the first plurality and the one or more additional pluralities comprises a distinct sample barcode sequence.
17. A composition for sample identification, comprising:
   a) a first plurality of complexes, each comprising a glycan-binding agent conjugated to a universal anchor,
   b) one or more additional pluralities of complexes each comprising a glycan-binding agent conjugated to a universal anchor.
18. The composition of embodiment 17, wherein the universal anchor is hybridized to a labeling oligonucleotide comprising a binding site for a primer, a sample barcode sequence and a capture sequence; and wherein the sample barcode sequence within each of the one or more additional pluralities comprises a substantially identical barcode sequence; and wherein the sample barcode sequence between the first plurality and the one or more additional pluralities comprises a distinct sample barcode sequence.
19. The composition of embodiment 17 or embodiment 18, wherein the conjugation of the glycan binding agent to the universal anchor comprises a lysine side amino group.
20. A composition for sample identification, comprising:
   a) a first plurality of complexes, each comprising a glycan-binding agent conjugated to a labeling oligonucleotide,
   b) one or more additional pluralities of complexes each comprising a glycan-binding agent conjugated to a labeling oligonucleotide.
21. The composition of embodiment 20, wherein the labeling oligonucleotide comprises a binding site for a primer, a sample barcode sequence and a capture sequence; wherein the sample barcode sequence within each of the one or more additional pluralities comprises a substantially identical barcode sequence; and wherein the sample barcode sequence between the first plurality and the one or more additional pluralities comprises a distinct sample barcode sequence.
22. The composition of embodiment 20 or 21, wherein the conjugation of the glycan binding agent to the labeling oligonucleotide comprises an IgG glycan as a site for oxime and hydrazine bioconjugation.
23. The composition of any one of embodiments 17-22, wherein the glycan-binding agent of the first plurality and the glycan binding agent of the one or more additional pluralities comprise a substantially identical glycan binding agent.
24. The composition of any one of embodiments 17-23, wherein the glycan-binding agent of the first plurality and the glycan binding agent of the one or more additional pluralities comprise a distinct glycan binding agent.
25. The composition of any one of embodiments 1-24, wherein the glycan-binding agent does not comprise an antibody.
26. The composition of any one of embodiments 5-25, wherein the capture sequence hybridizes to a complementary sequence on a capture polymer.
27. The composition of embodiment 26, wherein the capture polymer is immobilized to a solid substrate.
28. The composition of embodiment 27, wherein the substrate is a bead, a microfluidics bead, a slide, a multi-well plate or a chip.
29. The composition of any one of embodiments 5-28, wherein the binding site for a primer comprises a polynucleotide sequence of at or about 10 nucleotide bases that provides an annealing site for amplification of the universal anchor.
30. The composition of any one of embodiments 5-29, wherein the sample barcode sequence comprises a polynucleotide sequence of at least at or about 2 nucleotide bases.
31. The composition of any one of embodiments 5-30, wherein the sample barcode comprises a spatial barcode sequence.
32. The composition of any one of embodiments 1-31, wherein the glycan-binding agent comprises a carbohydrate binding agent.
33. The composition of any one of embodiments 1-32, wherein the glycan-binding agent comprises a glycan-binding protein (GBP).
34. The composition of any one of embodiments 1-33, wherein the glycan-binding agent comprises a lectin.
35. The composition of any one of embodiments 1-34, wherein the glycan-binding agent is derived from a plant, an animal, a fungus, a microbe, or a parasite.
36. The composition of any one of embodiments 1-35, wherein the glycan-binding agent is selected from concanavalin A (ConA), a wheat germ agglutinin (WGA), a pea lectin, a soybean lectin, a lentil lectin, a ricin, a bacterial toxin, an influenza virus hemagglutinin, a C-type lectin, an S-type lectin, a P-type lectin, or an I-type lectin.
37. The composition of any one of embodiments 1-36, wherein the glycan-binding agent of the first plurality and the one or more additional pluralities comprises concanavalin A (ConA).
38. The composition of any one of embodiments 1-36, wherein the glycan-binding agent of the first plurality and the one or more additional pluralities comprises wheat germ agglutinin (WGA).
39. The composition of any one of embodiments 1-36, wherein the glycan-binding agent of the first plurality comprises concanavalin A (ConA) and the glycan-binding agent of the one or more additional pluralities comprises wheat germ agglutinin (WGA).
40. The composition of any one of embodiments 1-39, further comprising a detectable label.
41. The composition of embodiment 40, wherein the detectable label is a tag or a fluorescent label.
42. The composition of any one of embodiments 1-41, wherein the glycan-binding agent binds to all or a portion of a ligand on the one or more target cells.
43. The composition of embodiment 42, wherein the ligand comprises all or a portion of a glycan on one or more target cells.
44. The composition of embodiment 42 or embodiment 43, wherein the ligand comprises a common core structure of a glycan.
45. The composition of embodiment 44, wherein the common core structure is a pentacaccharide.
46. The composition of embodiment 45, wherein the pentasaccharide is Man₃GlcNAc₂. 47. The composition of any one of embodiments 44-46, wherein the common core structure comprises an N-linked glycan.
48. The composition of any one of embodiments 44-46, wherein the common core structure comprises an O-linked glycan.
49. The composition of any one of embodiments 43-48, wherein the one or more target cells comprise a first cell type and a second cell type.
50. The composition of embodiment 49, wherein the first cell type and the second cell type comprise human cells.
51. The composition of embodiment 49, wherein the first cell type and the second cell type comprise non-human cells.
52. The composition of embodiment 49, wherein the first cell type comprises human cells and the second cell type comprises non-human cells.
53. The composition of any one of embodiments 43-52, wherein the one or more target cells comprise an immune cell, a non-immune cell, a hematopoietic cell, a blood cell, a stem cell, an epithelial cell, an endothelial cell, a nerve cell, a muscle cell, a fat cell, a bone cell, a reproductive cell, a lung cell, a cardiac cell, a tumor cell or a cancer cell.
54. The composition of any one of embodiments 43-53, wherein the one or more target cells comprise cancer cells.
55. The composition of any one of embodiments 43-54, wherein the one or more target cells comprise immune cells.
56. The composition of embodiment 55, wherein the immune cells comprise lymphocytes or a myeloid cells.
57. The composition of embodiment 55 or embodiment 56, wherein the immune cells comprise T cells, B cells, monocytes, basophils, eosinophils, neutrophils, or natural killer cells.
58. The composition of any one of embodiments 43-57, wherein the glycan is on the surface of a target cell.
59. The composition of any one of embodiments 43-57, wherein the glycan is on a membrane of a target cell.
60. The composition of any one of embodiments 43-57, wherein the glycan is in the intracellular space of a target cell.
61. The composition of any one of embodiments 43-60, wherein the glycan comprises a glycoconjugate, a glycoprotein, glycolipid, a proteoglycan, a galectin or a SIGLEC.
62. The composition of any one of embodiments 43-60, wherein the glycan comprises a differentiation marker.
63. The composition of any one of embodiments 43-62, wherein the glycan comprises an antigenic determinant.
64. The composition of any one of embodiments 8-63, wherein the glycan binding agent of the first plurality binds to all or a portion of a target on a first cell type, and the glycan binding agent of each of the one or more additional pluralities binds to all or a portion of a target on a second cell type.
65. The composition of embodiment 64 wherein the first cell type and the second cell type comprise human cells.
66. The composition of embodiment 64, wherein the first cell type and the second cell type comprise non-human cells.
67. The composition embodiment 64, wherein the first cell type comprises human cells and the second cell type comprises non-human cells.
68. The composition of any one of embodiments 1-67, wherein the first plurality comprises 2 or more glycan-binding agents conjugated to a universal anchor.
69. The composition of any one of embodiments 1-68, wherein the first plurality comprises between 1 to 100 cell glycan-binding agents conjugated to a universal anchor.
70. The composition of any one of embodiments 1-69, wherein the first plurality comprises 100 or more glycan-binding agents conjugated to a universal anchor.
71. The composition of any one of embodiments 1-70, wherein the first plurality comprises 1000 or more glycan-binding agents conjugated to a universal anchor.
72. The composition of any one of embodiments 8-71, wherein each of the one or more additional pluralities comprises 2 or more glycan-binding agents conjugated to a universal anchor.
73. The composition of any one of embodiments 8-72, wherein each of the one or more additional pluralities comprises between 1 to 100 glycan-binding agents conjugated to a universal anchor.
74. The composition of any one of embodiments 8-73, wherein each of the one or more additional pluralities comprises 100 or more glycan-binding agents conjugated to a universal anchor.
75. The composition of any one of embodiments 8-74, wherein each of the one or more additional pluralities comprises 1000 or more glycan-binding agents conjugated to a universal anchor.
76. The composition of any one of embodiments 8-75, wherein the one or more additional pluralities of comprises 1 plurality of complexes.
77. The composition of any one of embodiments 8-76, wherein the one or more additional pluralities comprises between 1 to 100 pluralities of complexes.
78. The composition of any one of embodiments 8-77, wherein the one or more additional pluralities comprises 10 or more pluralities of complexes.
79. The composition of any one of embodiments 8-78, wherein the one or more additional pluralities comprises 20 or more pluralities of complexes.
80. The composition of any one of embodiments 8-79, wherein the one or more additional pluralities comprises 30 or more pluralities of complexes.
81. The composition of any one of embodiments 8-80, wherein the one or more additional pluralities comprises 40 or more pluralities of complexes.
82. The composition of any one of embodiments 8-81, wherein the one or more additional pluralities comprises 50 or more pluralities of complexes.
83. The composition of any one of embodiments 8-82, wherein the one or more additional pluralities comprises 100 or more pluralities of complexes.
84. The composition of any one of embodiments 8-83, wherein the one or more additional pluralities comprises 1000 or more pluralities of complexes.
85. A kit, comprising the composition of any of embodiments 1-84.
86. The kit of embodiment 85, wherein the first plurality and the one or more additional pluralities are packaged separately.
87. A kit comprising,
a) a plurality of substantially identical complexes comprising a glycan-binding agent and a universal anchor;
b) a first plurality of labeling oligonucleotides comprising a binding site for a primer, a substantially identical sample barcode sequence and a capture sequence; and
c) one or more additional pluralities of labeling oligonucleotides, wherein each labeling oligonucleotide comprises a binding site for a primer, a substantially identical sample barcode sequence and a capture sequence;
   wherein the sample barcode sequence between each of the first and one or more additional pluralities of labeling oligonucleotides comprises a distinct sample barcode sequence from the first plurality of labeling oligonucleotides or any other additional pluralities of labeling oligonucleotides.
88. A kit comprising,
a) a first plurality of complexes comprising substantially identical glycan-binding agents conjugated to a universal anchor;
b) one or more additional pluralities of complexes, each plurality comprising a substantially identical glycan-binding agent conjugated to a universal anchor; wherein the glycan binding agent between the first plurality and the one or more additional pluralities comprises a distinct glycan binding agent;
c) a first plurality of labeling oligonucleotides comprising a binding site for a primer, a substantially identical sample barcode sequence and a capture sequence; and
d) one or more additional pluralities of labeling oligonucleotides comprising a binding site for a primer, a substantially identical sample barcode sequence and a capture sequence;
   wherein the sample barcode sequence between each of the first and one or more additional pluralities of labeling oligonucleotides comprises a distinct sample barcode sequence.
89. The kit of embodiment 87 or embodiment 88, wherein each component is packaged separately.
90. A method of generating a pool of samples for sequencing, comprising
   a) generating a first sample by contacting the first plurality of complexes of any of embodiments 1-84 with a first plurality of target cells comprising one or more glycans, wherein the first plurality of complexes bind to the one or more glycans,
   b) generating one or more additional samples by contacting the one or more additional pluralities of complexes of any of embodiments 8-84 with one or more additional pluralities of target cells each comprising one or more glycans, wherein the one or more additional pluralities of complexes bind to the one or more glycans, and
   c) pooling the first and the one or more additional samples.
91. A method of identifying a sample, comprising
   a) generating a first plurality of samples by contacting the first plurality of complexes of any of embodiments 1-84 with a first plurality of target cells comprising one or more glycans, wherein the first plurality of complexes bind to the one or more glycans,
   b) generating one or more additional pluralities of samples by contacting the one or more additional pluralities of complexes of any of embodiments 8-84 with one or more additional pluralities of target cells each comprising one or more glycans, wherein the one or more additional pluralities of complexes bind to the one or more glycans,
   c) pooling the first and the one or more additional pluralities of samples,
   d) partitioning each sample from the pool of first and one or more additional pluralities of samples into a plurality of partitions, wherein each partition of the plurality of partitions comprises a single cell associated with a complex,
   e) within the partition, hybridizing the capture sequence on the complex associated with a capture polymer on a solid substrate,
   f) obtaining sequence information from the pool of first and one or more additional pluralities of samples to identify the first and one or more additional pluralities of target cells from the pool generated in (c).
92. A method of identifying a first cell type and one or more additional cell types from a mixture of cells, comprising
   a) generating a first plurality of samples by contacting the first plurality of complexes of any of embodiments 1-84 with a first plurality of target cells comprising one or more glycans, wherein the first plurality of complexes bind to the one or more glycans,
   b) generating one or more additional pluralities of samples by contacting the one or more additional pluralities of complexes of any of embodiments 8-84 with one or more additional pluralities of target cells each comprising one or more glycans, wherein the one or more additional pluralities of complexes bind to the one or more glycans,
   c) pooling the first and the one or more additional pluralities of samples,
   d) partitioning each sample from the pool of first and one or more additional pluralities of samples into a plurality of partitions, wherein each partition of the plurality of partitions comprises a single cell associated with a complex,
   e) within the partition, hybridizing the capture sequence on the complex with a capture polymer on a solid substrate
   f) obtaining sequence information from the pool of first and one or more additional pluralities of samples to identify the first and one or more additional pluralities of target cells from the pool generated in (c).
93. The method of embodiment 92, wherein the first plurality of target cells and the one or more additional pluralities of target cells are selected from the group comprising human cells, non-human cells, healthy cells, cells associated with a disease or disorder, cancer cells, non-cancer cells, cells from the same subject, cells from a different subject, activated cells, non-activated cells, fresh cells, fixed cells, permeabilized cells, cells from a human subject or cells from a cell line.
94. The method of embodiment 93, wherein the cancer is selected from leukemia, polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia, myelodysplasia, sarcoma, carcinoma, fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancer, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyngioma, ependymoma, pinealoma, hemangioblastoma, acoustic neuroma, oligodendroglioma, meningioma, melanoma, neuroblastoma and retinoblastoma).
95. The method of any one of embodiments 86-94, wherein the generating one or more additional pluralities of samples further comprises contacting the target cells with an antibody comprising a labeling oligonucleotide.
96. The method of any one of embodiments 86-95, further comprising contacting the target cells with an antibody comprising an oligonucleotide and/or a polyethylene glycol (PEG) conjugated to a glycan present on the antibody prior to pooling.
97. The method of embodiment 95 or embodiment 96, further comprising blocking the antibody with an unconjugated lectin.
98. The method of any of embodiments 86-97, comprising lysing the cell after hybridizing the capture sequence to the capture polymer.
99. A method of identifying a sample through sequential hybridization, comprising
   a) contacting a first plurality of complexes each comprising a substantially identical glycan-binding agent conjugated to a universal anchor with a first plurality of target cells comprising one or more glycans, wherein the first plurality of complexes bind to the one or more glycans,
   b) reversibly hybridizing the first plurality of complexes to a complementary sequence on a first labeling oligonucleotide comprising a binding site for a primer, a sample barcode sequence, a capture sequence, and a detectable label,
   c) detecting the detectable label,
   d) de-hybridizing the first plurality of complexes,
   e) reversibly hybridizing the first plurality of complexes to a complementary sequence on a second labeling oligonucleotide comprising a binding site for a primer, a sample barcode sequence, a capture sequence, and a detectable label,
   f) detecting the detectable label
   g) identifying the sample based on the detectable labels.
100. A method of identifying a sample through sequential hybridization, comprising
   a) contacting a first plurality of complexes each comprising a substantially identical glycan-binding agent conjugated to a universal anchor with a first plurality of target cells comprising one or more glycans, wherein the first plurality of complexes bind to the one or more glycans,
   b) contacting one or more additional pluralities of complexes, each plurality comprising a distinct glycan-binding agent conjugated to a universal anchor, with the first plurality of target cells comprising one or more glycans, wherein the one or more additional pluralities of complexes bind to the one or more glycans,
   c) incubating the first plurality of complexes and the one or more additional pluralities of complexes with a first labeling oligonucleotide comprising a binding site for a primer, a sample barcode sequence, a capture sequence, and a detectable label, under conditions to reversibly hybridize the first labeling oligonucleotide to the universal anchor,
   c) detecting the detectable label,
   d) de-hybridizing the first plurality of complexes and the one or more additional pluralities of complexes,
   e) incubating the first plurality of complexes and the one or more additional pluralities of complexes with a second labeling oligonucleotide comprising a binding site for a primer, a sample barcode sequence, a capture sequence, and a detectable label, under conditions to reversibly hybridize the second labeling oligonucleotide to the universal anchor,
   f) detecting the detectable label
   g) identifying the sample based on the detectable labels.
101. The method of embodiment 99 or embodiment 100, wherein the sample barcode sequence on the second labeling oligonucleotide is different than the sample barcode sequence from the first labeling oligonucleotide.
102. The method of embodiment 98 or embodiment 99, wherein the detectable label on the second labeling oligonucleotide is different than the detectable label from the first labeling oligonucleotide.
103. A method of associating presence or abundance of a glycan with a location on a tissue sample, comprising:
   delivering the first plurality of complexes of any of embodiments 1-84 to a tissue sample affixed to a support, under conditions to specifically bind the first plurality of complexes to the glycan at a location of the tissue sample
   sequencing all or a portion of the first plurality of complexes, and using the determined sequence to associate presence or abundance of the target biological molecule with the location of the tissue sample.
104. The method of embodiment 103, further comprising:
   delivering the one or more additional pluralities of complexes of any of embodiments 8-84 to a tissue sample affixed to a support, under conditions to specifically bind the one or more additional pluralities of complexes to the glycan at a location of the tissue sample
   sequencing all or a portion of the one or more additional pluralities of complexes, and using the determined sequence to associate presence or abundance of the target biological molecule with the location of the tissue sample.
105. The method of any of embodiments 90-104, wherein the sample comprises a sub-cellular component.
106. The method of embodiments 105, wherein the sub-cellular component is isolated.
107. The method of embodiments 105 or embodiments 106, wherein the sub-cellular component is selected from the group consisting of a nucleus, ribosome, an endoplasmic reticulum, a Golgi apparatus, a cytoskeleton, a mitochondria, a vacuole and a vesicle.
108. The method of any of embodiments 105-107, wherein the sub-cellular component is a nucleus.
109. A method of determining the progression of a disease or disorder, comprising:
   (a) contacting the first plurality of complexes of any of any of embodiments 8-84 to, and optionally the one or more additional pluralities of complexes of any of embodiments 8-84 to, to a tissue sample suspected of having a disease or disorder affixed to a support, under conditions to specifically bind the first plurality and one or more additional pluralities of complexes to a glycan on the tissue sample,
   (b) sequencing all or a portion of the first plurality of complexes, and all or a portion of the one or more additional pluralities of complexes,
   (c) determining the progression of the disease or disorder based on the expression profiles of the glycan expressed on the tissue sample.
110. The method of any of embodiments 99-109, wherein the generating one or more additional pluralities of samples further comprises contacting the tissue sample with an antibody comprising a labeling oligonucleotide.
111. The method of any one of embodiments 99-110, further comprising contacting the tissue sample with an antibody comprising an oligonucleotide and/or a polyethylene glycol (PEG) conjugated to a glycan present on the antibody prior to pooling.
112. The method of embodiment 110 or embodiment 111, further comprising blocking the antibody with an unconjugated lectin.

### EXAMPLES

The following examples are included for illustrative purposes only and are not intended to limit the scope of the invention.

### Example 1 Assessment of Lectin-Biotin Conjugates as Universal Reagents for Sample Multiplexing

Given the complexity of direct conjugation of lectins, this Example describes the use of lectin-biotin conjugates in single cell sequencing methods, as an initial approach for examining the ability to use lectins for the identification of a sample of origin for a cell from a pool of distinct samples. In this Example, glycan based surface markers on the cells of each distinct sample were labeled with a unique lectin-biotin conjugate and an oligonucleotide-conjugated anti-biotin antibody prior to pooling, and compared to cells labeled with commercially available antibody based hashtag oligonucleotides (HTOs) that identify protein markers on the cell surface.

### 1.A Generation of Exemplary Oligonucleotide-Conjugated Anti-Biotin Antibodies

Exemplary oligonucleotide-conjugated anti-biotin antibodies were prepared as follows. Anti-biotin antibodies (BioLegend Cat #409002) were treated with 1M DTT for 30 minutes, desalted via a column packed with Sephadex G-25 Medium resins (Cytiva Cat #17003302), immediately reacted with 8 molar equivalence of DBCO-PEG-Maleimide (Click Chemistry Tools Cat #A108P) for 30 minutes, and desalted again with a column packed with Sephadex G-25 Medium resins (Cytiva Cat #17003302). The resulting DBCO-modified antibodies was then mixed with 1 molar equivalence of an exemplary 5′-azide modified oligonucleotide, ABT1-ABT5, overnight. Exemplary oligonucleotides ABT1-ABT5 are outlined in Table E1.

TABLE E1
Exemplary oligonucleotides for generation of conjugated
anti-biotin antibodies

| Exemplary Anti-Biotin | |
|---|---|
| Oligonucleotide Sequence | SEQ ID NO |
| ABT1 | SEQ ID NO: 1 |
| ABT2 | SEQ ID NO: 2 |
| ABT3 | SEQ ID NO: 3 |
| ABT4 | SEQ ID NO: 4 |
| ABT5 | SEQ ID NO: 5 |

A “phosphorothioate (PS) bond” is indicated with a * in the above sequences and refers to a phosphorothioate (PS) bond between the two nucleotide residues. A phosphorothioate bond substitutes a sulfur atom for a non-bridging oxygen atom in the phosphate backbone of an oligo.

The reaction mixture was loaded on a SEC column packed with Superdex 200 resin (Cytiva Cat #17104304) and eluted with 1×PBS pH 7.2 with 0.09% NaN3 for purification. The fractions collected from the purification were analyzed by isoelectric focusing gel electrophoresis before pooling solutions that contained the target conjugate. The pooled solutions were concentrated by centrifugal filters and diluted to the desirable concentration for the subsequent testing.

### 1.B Generation of Exemplary Lectin-Biotin Conjugates.

Biotinylated lectins Concanavalin A (Con A), Sambucus Nigra Lectin (SNA, EBL), Maackia Amurensis Lectin II (MAL II), Ulex Europaeus Agglutinin I (UEA I), and Aleuria Aurantia Lectin (AAL) (VectorLabs, Cat #B-1005-5, B-1305-2, B-1265-1, B-1065-2 and B-1395-1, respectively) were pooled in equal concentrations to form exemplary lectin-biotin conjugates.

### 1.C Assessment of Cross-Species Compatibility and Activation States of Exemplary Lectin-Biotin Conjugates in Fresh and Fixed Immune Cells.

Exemplary lectin-biotin conjugates were assessed for their ability to identify multiple species using a combination of human and murine cells, and for their ability to identify distinct activation states in both fresh and fixed samples.

Mouse bone marrow cells (BM) and Thymocytes (Thy) were isolated from C57BL/6 mouse on the day of the experiment. Briefly, one mouse was euthanized by CO2 inhalation followed by cervical dislocation. Next, both femur bones were harvested, cleaned and the ends were cut using sharp scissors. Bone Marrow cells were collected by flushing 10 mL of RPMI complete media (ThermoFisher Cat #A1049101; Cat #26170043; Cat #21985023; Cat #15140122) through the marrow using a 25 g needle. For thymocyte isolation, the thymus was harvested and washed with PBS for RBC removal, and cells were collected in a petri-dish by mechanic disruption using a 70 μm cell strainer and ice-cold RPMI media. Human peripheral blood mononuclear cells (PBMC) were isolated by gradient separation using Lymphopure (BioLegend Cat #426202) and SepMate-50 tubes (Stem Cell Cat #85460). Heparinized blood collected on day 1 of isolation was diluted in 1 volume of PBS containing 2% FBS, and carefully overlaid on top of Lymphopure. SepMate tubes were centrifuged at 1200 rcf for 10 minutes allowing PBMC enrichment. Cells were collected and washed in Cell Staining buffer (BioLegend Cat #420201).

In addition to cross species reactivity, lectin-biotin conjugates were also assessed across distinct activation states. For this, fresh human PBMCs were isolated as before and cultured with Phytohaemagglutinin-L (PHA) (Sigma Cat #1 1249738001) for 72 h or Cell Activation Cocktail (CAC) (BioLegend Cat #423301) for 6 h. RPMI media alone was used as unstimulated control.

Treated PBMCs (PBMC_PHA and PBMC_CAC), unstimulated PBMCs, mouse Bone Marrow (BM), and mouse thymocytes (Thy) were split into 3 separate aliquots. Each aliquot was labeled with 0.25 μg of an exemplary protein identifying antibody based HTO, 0.125 μg an exemplary lectin-biotin conjugate, and an oligonucleotide conjugated anti-biotin antibody as described in Example 1.A, for 30 minutes on ice (4° C.) or 15 minutes at room temperature, allowing the oligonucleotide conjugated anti-biotin antibody to bind to the lectin-biotin conjugate. Labeling of cells is outlined in Table E2 below. Cells were washed twice with Cell Staining buffer after each staining step. After the final wash, each of the 3 separate aliquots of each cell type was pooled, samples were counted, cell concentration was normalized to 2e6 cells and equal volumes were pooled in a separate tube.

For experiments involving fixed samples, cell preparation was performed similarly, but included a fixation step before lectin and HTO labeling. Cell fixation was achieved by incubating cells in 1% PFA in PBS for 20 min at room temperature, followed by two buffer washes in PBS+1% FBS+0.09% azide.

TABLE E2
Cell labeling for assessment of lectin-biotin conjugates

| | | Exemplary | |
|---|---|---|---|
| | | Oligonucleotide | |
| | HTO | Conjugated to | |
| | barcode | Anti-Biotin | |
| | sequence | Antibody | |
| | (SEQ | Sequence | |
| Sample | ID NO) | (SEQ ID NO) | Lectin-Biotin Conjugate |
| PBMC_ | A0251 | ABT1 | Concanavalin A (Con A), |
| unstim | (BioLegend | (SEQ ID NO: 1) | Sambucus Nigra Lectin |
| | Cat# 394601) | | (SNA, EBL), Maackia |
| | (SEQ ID | | Amurensis Lectin II (MAL |
| | NO: 6) | | II), Ulex Europaeus |
| | | | Agglutinin I (UEA I), and |
| | | | Aleuria Aurantia Lectin |
| | | | (AAL) (VectorLabs, Cat#B- |
| | | | 1005-5, B-1305-2, B-1265-1, |
| | | | B-1065-2 and B-1395-1) |
| PBMC_ | A0252 | ABT2 | Concanavalin A (Con A), |
| CAC_6h | (BioLegend | (SEQ ID NO: 2) | Sambucus Nigra Lectin |
| | Cat# 394603) | | (SNA, EBL), Maackia |
| | (SEQ ID | | Amurensis Lectin II (MAL |
| | NO: 7) | | II), Ulex Europaeus |
| | | | Agglutinin I (UEA I), and |
| | | | Aleuria Aurantia Lectin |
| | | | (AAL) (VectorLabs, Cat#B- |
| | | | 1005-5, B-1305-2, B-1265-1, |
| | | | B-1065-2 and B-1395-1) |
| PBMC_ | A0253 | ABT3 | Concanavalin A (Con A), |
| PHA_3d | (BioLegend | (SEQ ID NO: 3) | Sambucus Nigra Lectin |
| | Cat# 394605) | | (SNA, EBL), Maackia |
| | (SEQ ID | | Amurensis Lectin II (MAL |
| | NO: 8) | | II), Ulex Europaeus |
| | | | Agglutinin I (UEA I), and |
| | | | Aleuria Aurantia Lectin |
| | | | (AAL) (VectorLabs, Cat#B- |
| | | | 1005-5, B-1305-2, B-1265-1, |
| | | | B-1065-2 and B-1395-1) |
| Mouse_ | A0301 | ABT4 | Concanavalin A (Con A), |
| thymus | (BioLegend | (SEQ ID NO: 4) | Sambucus Nigra Lectin |
| | Cat# 155801) | | (SNA, EBL), Maackia |
| | (SEQ ID | | Amurensis Lectin II (MAL |
| | NO: 9) | | II), Ulex Europaeus |
| | | | Agglutinin I (UEA I), and |
| | | | Aleuria Aurantia Lectin |
| | | | (AAL) (VectorLabs, Cat#B- |
| | | | 1005-5, B-1305-2, B-1265-1, |
| | | | B-1065-2 and B-1395-1) |
| Mouse_ | A0302 | ABT5 | Concanavalin A (Con A), |
| BM | (BioLegend | (SEQ ID NO: 5) | Sambucus Nigra Lectin |
| | Cat# 155803) | | (SNA, EBL), Maackia |
| | (SEQ ID | | Amurensis Lectin II (MAL |
| | NO: 10) | | II), Ulex Europaeus |
| | | | Agglutinin I (UEA I), and |
| | | | Aleuria Aurantia Lectin |
| | | | (AAL) (VectorLabs, Cat#B- |
| | | | 1005-5, B-1305-2, B-1265-1, |
| | | | B-1065-2 and B-1395-1) |
| PBMC_ | A0251 | ABT1 | Concanavalin A (Con A), |
| unstim_ | (BioLegend | (SEQ ID NO: 1) | Sambucus Nigra Lectin |
| fixed | Cat# 394601) | | (SNA, EBL), Maackia |
| | (SEQ ID | | Amurensis Lectin II (MAL |
| | NO: 6) | | II), Ulex Europaeus |
| | | | Agglutinin I (UEA I), and |
| | | | Aleuria Aurantia Lectin |
| | | | (AAL) (VectorLabs, Cat#B- |
| | | | 1005-5, B-1305-2, B-1265-1, |
| | | | B-1065-2 and B-1395-1) |
| PBMC_ | A0252 | ABT2 | Concanavalin A (Con A), |
| CAC_ | (BioLegend | (SEQ ID NO: 2) | Sambucus Nigra Lectin |
| 6h_fixed | Cat# 394603) | | (SNA, EBL), Maackia |
| | (SEQ ID | | Amurensis Lectin II (MAL |
| | NO: 7) | | II), Ulex Europaeus |
| | | | Agglutinin I (UEA I), and |
| | | | Aleuria Aurantia Lectin |
| | | | (AAL) (VectorLabs, Cat#B- |
| | | | 1005-5, B-1305-2, B-1265-1, |
| | | | B-1065-2 and B-1395-1) |
| PBMC_ | A0253 | ABT3 | Concanavalin A (Con A), |
| PHA_ | (BioLegend | (SEQ ID NO: 3) | Sambucus Nigra Lectin |
| 3d_fixed | Cat# 394605) | | (SNA, EBL), Maackia |
| | (SEQ ID | | Amurensis Lectin II (MAL |
| | NO: 8) | | II), Ulex Europaeus |
| | | | Agglutinin I (UEA I), and |
| | | | Aleuria Aurantia Lectin |
| | | | (AAL) (VectorLabs, Cat#B- |
| | | | 1005-5, B-1305-2, B-1265-1, |
| | | | B-1065-2 and B-1395-1) |
| Mouse_ | A0304 | ABT4 | Concanavalin A (Con A), |
| thymus_ | (BioLegend | (SEQ ID NO: 4) | Sambucus Nigra Lectin |
| fixed | Cat# 155807) | | (SNA, EBL), Maackia |
| | (SEQ ID | | Amurensis Lectin II (MAL |
| | NO: 11) | | II), Ulex Europaeus |
| | | | Agglutinin I (UEA I), and |
| | | | Aleuria Aurantia Lectin |
| | | | (AAL) (VectorLabs, Cat#B- |
| | | | 1005-5, B-1305-2, B-1265-1, |
| | | | B-1065-2 and B-1395-1) |
| Mouse_ | A0302 | ABT5 | Concanavalin A (Con A), |
| BM_ | (BioLegend | (SEQ ID NO: 5) | Sambucus Nigra Lectin |
| fixed | Cat# 155803) | | (SNA, EBL), Maackia |
| | (SEQ ID | | Amurensis Lectin II (MAL |
| | NO: 10) | | II), Ulex Europaeus |
| | | | Agglutinin I (UEA I), and |
| | | | Aleuria Aurantia Lectin |
| | | | (AAL) (VectorLabs, Cat#B- |
| | | | 1005-5, B-1305-2, B-1265-1, |
| | | | B-1065-2 and B-1395-1) |

Fresh pooled cells and fixed pooled cells were separately loaded onto a 10× chromium channel, and library preparation was performed according to standard cell hashing protocol. Briefly, Gel Emulsion and in-drop Reverse Transcription was used to generate first strand cDNA product. After Dynabead cleanup, second strand cDNA for lectin-biotin conjugates or HTO protein fractions were amplified using specific primers. After size-selection cleanup, sequencing-ready library was created using indexing primers. The libraries were pooled and sequenced on Illumina NovaSeq platform. The resulting fastq were used to generate count matrix using Cellranger count software.

Exemplary lectin-biotin conjugates bound to oligonucleotide containing anti-biotin antibodies and HTOs were demultiplexed by a three-step evaluation procedure where positive and negative populations were identified based on the staining level of each lectin-biotin conjugate bound to oligonucleotide containing anti-biotin antibody and HTO, doublets were defined as cells that were positive for more than one lectin-biotin conjugate bound to oligonucleotide containing anti-biotin antibody and HTO, and negative populations were defined as not positive for any lectin-biotin conjugate bound to oligonucleotide containing anti-biotin antibody and HTO Results were merged back at single cell level for comparison and evaluation.

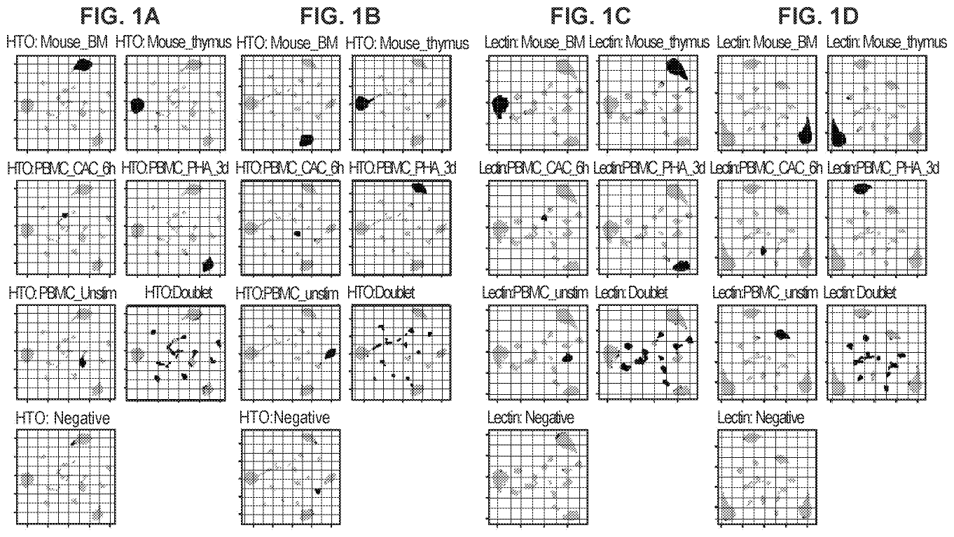

FIGS. 1A-1D show uniform manifold approximation and projection (UMAP) plots of individual cell populations of mouse bone marrow (mBM), mouse thymocytes (mThy), hPBMC_PHA, hPBMC_CAC, hPBMC_rest, for fresh cells demultiplexed by HTOs (FIG. 1A), fixed cells demultiplexed by with HTOs (FIG. 1B), fresh cells demultiplexed by exemplary lectin-biotin conjugates (FIG. 1C) and fixed demultiplexed by exemplary lectin-biotin conjugates (FIG. 1D). Cells were clustered based on the staining level of the indicated lectin-biotin conjugate bound to oligonucleotide containing anti-biotin antibody or HTO. Representative data for doublets and negatively stained cells is depicted as well. As shown in FIGS. 1A-1D, both the HTOs and lectin-biotin conjugates were capable of separating the classes of cells, based on the conditions described above, to a comparable level.

These results demonstrate the ability of exemplary lectin-biotin conjugates to stain multiple cell types across human and murine species, using both fresh and fixed samples at optimized concentrations compared to antibody based HTOs. These results further demonstrate the ability of exemplary lectin-biotin conjugates to identify distinct activation states in immune cells.

### 1.D Assessment of Exemplary Lectin-Biotin Conjugate Labeling of Cancer Cell Lines.

Exemplary lectin-biotin conjugates were further assessed for their ability to identify multiple cancer cell lines. Murine skin melanoma cells (B16-F0; ATCC Cat #CRL-6322), murine interleukin-3 dependent pro-B cells (Ba/F3; DSMZ Cat #ACC 300), murine myoblast cells (C2C12; ATCC Cat #CRL-1772), murine fibroblasts (L929; ATCC Cat #CCL-1) and murine embryonic fibroblasts (NIH/3T3; ATCC Cat #CRL-1658) were cultured in T25 flasks according to manufacturer specifications (Corning Cat #430639). Once cells reached confluency but still under log phase, cells were collected and labeled with exemplary HTOs and lectin-biotin conjugates and oligonucleotide-anti-biotin antibodies, as outlined in Table E3.

TABLE E3
Labeling of cancer cell lines

| | | Exemplary | |
|---|---|---|---|
| | | Oligonucleotide | |
| | | Conjugated to | |
| | | Anti-Biotin | |
| | | Antibody | |
| | HTO (SEQ | Sequence | |
| Sample | ID NO) | (SEQ ID NO) | Lectin-Biotin Conjugate |
| B16- | A0301 | ABT1 | Concanavalin A (Con A), |
| F0 | (BioLegend | (SEQ ID NO: | Sambucus Nigra Lectin (SNA, |
| | Cat# | 1) | EBL), Maackia Amurensis Lectin |
| | 155801) | | II (MAL II), Ulex Europaeus |
| | (SEQ ID | | Agglutinin I (UEA I), and Aleuria |
| | NO: 9) | | Aurantia Lectin (AAL) |
| | | | (VectorLabs, Cat#B-1005-5, B- |
| | | | 1305-2, B-1265-1, B-1065-2 and |
| | | | B-1395-1) |
| Ba/F3 | A0302 | ABT2 | Concanavalin A (Con A), |
| | (BioLegend | (SEQ ID NO: | Sambucus Nigra Lectin (SNA, |
| | Cat# | 2) | EBL), Maackia Amurensis Lectin |
| | 155803) | | II (MAL II), Ulex Europaeus |
| | (SEQ ID | | Agglutinin I (UEA I), and Aleuria |
| | NO: 10) | | Aurantia Lectin (AAL) |
| | | | (VectorLabs, Cat#B-1005-5, B- |
| | | | 1305-2, B-1265-1, B-1065-2 and |
| | | | B-1395-1) |
| C2C12 | A0303 | ABT3 | Concanavalin A (Con A), |
| | (BioLegend | (SEQ ID NO: | Sambucus Nigra Lectin (SNA, |
| | Cat# | 3) | EBL), Maackia Amurensis Lectin |
| | 155805) | | II (MAL II), Ulex Europaeus |
| | (SEQ ID | | Agglutinin I (UEA I), and Aleuria |
| | NO: 12) | | Aurantia Lectin (AAL) |
| | | | (VectorLabs, Cat#B-1005-5, B- |
| | | | 1305-2, B-1265-1, B-1065-2 and |
| | | | B-1395-1) |
| L929 | A0304 | ABT4 | Concanavalin A (Con A), |
| | (BioLegend | (SEQ ID NO: | Sambucus Nigra Lectin (SNA, |
| | Cat# | 4) | EBL), Maackia Amurensis Lectin |
| | 155807) | | II (MAL II), Ulex Europaeus |
| | (SEQ ID | | Agglutinin I (UEA I), and Aleuria |
| | NO: 11) | | Aurantia Lectin (AAL) |
| | | | (VectorLabs, Cat#B-1005-5, B- |
| | | | 1305-2, B-1265-1, B-1065-2 and |
| | | | B-1395-1) |
| NIH/ | A0305 | ABT5 | Concanavalin A (Con A), |
| 3T3 | (BioLegend | (SEQ ID NO: | Sambucus Nigra Lectin (SNA, |
| | Cat# | 5) | EBL), Maackia Amurensis Lectin |
| | 155809) | | II (MAL II), Ulex Europaeus |
| | (SEQ ID | | Agglutinin I (UEA I), and Aleuria |
| | NO: 13) | | Aurantia Lectin (AAL) |
| | | | (VectorLabs, Cat#B-1005-5, B- |
| | | | 1305-2, B-1265-1, B-1065-2 and |
| | | | B-1395-1) |

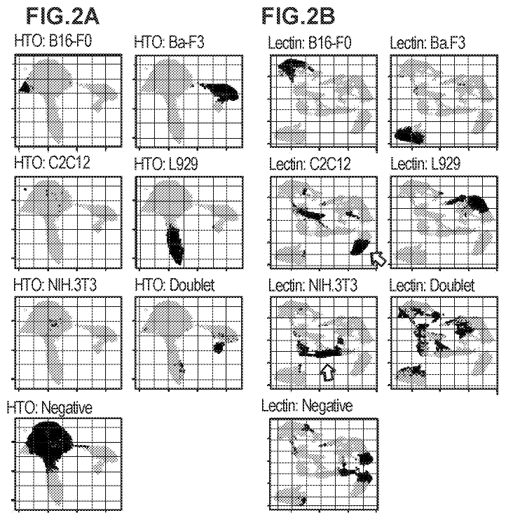

Cells were pooled and loaded on one 10× chromium channel, and library preparation, demultiplexing and results evaluated as described in Example 1.C above. FIGS. 2A and 2B show uniform manifold approximation and projection (UMAP) plots of individual cell populations of B16-F0, Ba/F3, C2C, 12, 1929 and NIH/3T3 cells labeled with HTOs (FIG. 2A), or with exemplary lectin-biotin conjugates (FIG. 2B). Cells were clustered based on the staining level of the indicated HTO or exemplary lectin-biotin conjugates. Representative data for doublets and negatively stained cells is depicted as well.

As shown in FIGS. 2A and 2B, HTO labeled cells resulted in a higher number of negative or non-assigned cells, and only lectin-biotin conjugates were capable of identifying C2C12 and NIH-3T3 cells (arrows). These results suggest the improved affinity of lectin-based hashing for cancer cell lines.

1.E Assessment of Transcriptome Effects in Cells Labeled with Exemplary Lectin-Biotin Conjugates.

To evaluate the effect of lectin-biotin conjugate labeling on cell stimulation, PBMCs with or without CAC treatment (CAC or unstim, described in Section 1. C) were labeled with exemplary lectin-biotin conjugates for 6 hours, or left unlabeled as control. The resulting cells were pooled and processed as described in Section 1.C above.

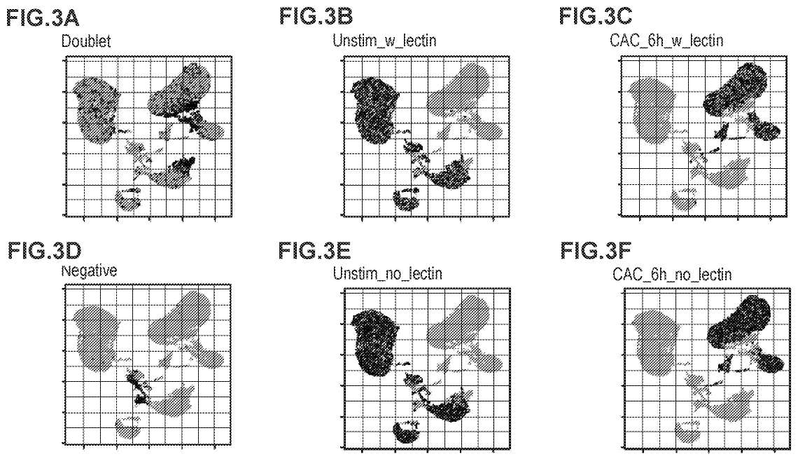

FIGS. 3A-3F show uniform manifold approximation and projection (UMAP) plots of individual cell populations of the unstim_with_lectin, unstim_no_lectin, CAC_6 h_with_lectin and CAC_6 h_no_lectin. As shown in FIG. 3C and FIG. 3F, cells stimulated with CAC_6 h displayed distinct clusters compared to cells not stimulated with CAC_6 h, as shown in FIG. 3B and FIG. 3E. Both the unstimulated and CAC treated lectin-biotin conjugate labeled groups show similar patterns to those of their respective non-lectin treated control groups.

These results suggest that the above described lectin-biotin conjugate staining method does not induce changes in activation state that would result from alteration of the transcriptome.

### 1.F Assessment of Exemplary Lectin-Biotin Conjugate Labeling of Isolated Nuclei.

Exemplary lectin-biotin conjugates were further assessed for their ability to identify sub-cellular components.

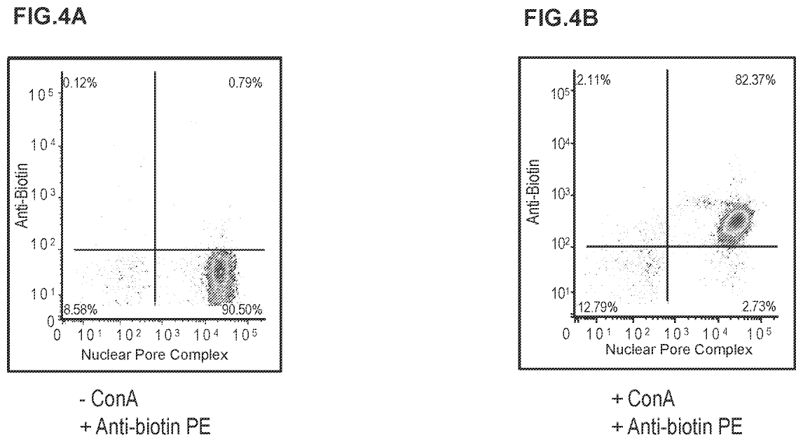

Nuclei were obtained from freshly isolated PBMCs by selective permeabilization of surface membrane using 0.025% NP-40 in PBS supplemented with 10 mM Tris-HCl (pH 7.4), 10 mM NaCl and 3 mM MgCl2. Lysis was stopped by adding 4 volumes of nuclei wash buffer (1% BSA in PBS) followed by centrifugation. Isolated nuclei were stained on ice for 30 min with Nuclear Pore Complex (AF647) (BioLegend cat #682203) in the absence of biotinylated ConA (−ConA; FIG. 4A) or the presence of biotinylated ConA (+ConA; FIG. 4B). Biotin detection was performed using anti-biotin PE (BioLegend cat #409003), and identification was performed using flow cytometric analysis.

As shown in FIG. 4B, exemplary lectin-biotin conjugates were capable of staining isolated nuclei, as shown by the number of cells positive for anti-Biotin PE in the upper right quadrant of the graph, whereas the absence of exemplary lectin-biotin conjugates, as shown in FIG. 4A, does not result in positive anti-Biotin PE detection.

These results demonstrate the ability of exemplary lectin-biotin conjugates to stain subcellular components.

### Example 2. Assessment of the Binding Profiles of Multiple Exemplary Lectins

This example describes the assessment of different lectins for sample identification through characterization of distinct staining patterns between cell samples.

Twenty-one biotinylated plant lectins with different specificities (Vector Labs, Table E4) were tested by flow cytometry for their ability to bind different cells, including human lymphoid white blood cells (LWB) or mouse spleen lymphoid cells. Briefly, human LWB cells were lysed with RBC lysis buffer IX (BioLegend Cat #420302) and individually stained with different concentrations of exemplary lectins in a modified cell staining buffer. Cells were then washed and stained with anti-Biotin PE stain (clone 1D4-C5 BioLegend Cat number 409004) and analyzed by flow cytometry. For mouse spleen lymphoid cells from BALB/c and C57BL/6 strains, spleens were removed and transferred to individual petri dishes containing cold PBS buffer. After chopping using scissors, organ pieces were transferred to a 40 uM mash and mechanically dissociated using a syringe plunger. Cell suspensions were transferred to a 50 mL tube, centrifuged, and supernatant removed. Cell pellets were resuspended into 5 mL RBC lysis buffer IX for 5 minutes and washed with lectin staining buffer as described above.

TABLE E4
Exemplary lectins

| CATALOG # | LECTIN | SPECIFICITY |
|---|---|---|
| B-1395-1 | Aleuria Aurantia Lectin (AAL) | Fucose |
| | Biotinylated | |
| B-1325-2 | Lotus Tetragonolobus Lectin (LTL) | Fucose |
| | Biotinylated | |
| B-1065-2 | Ulex Europaeus Agglutinin I (UEA I) | Fucose |
| | Biotinylated | |
| B-1045-5 | Lens Culinaris Agglutinin (LCA) | Fucose (N-type) |
| | Biotinylated | |
| B-1055-5 | Pisum Sativum Agglutinin (PSA) | Fucose (N-type) |
| | Biotinylated | |
| B-1145-5 | Erythrina Cristagalli Lectin (ECL, | Galactose (N-type) |
| | ECA) Biotinylated | |
| B-1155-5 | Jacalin Biotinylated | Galactose (O-type) |
| B-1075-5 | Peanut Agglutinin (PNA) Biotinylated | Galactose (O-type, |
| | | glycolipids) |
| B-1105-2 | Griffonia (Bandeiraea) Simplicifolia | Galactose, N- |
| | Lectin I (GAL I, BSL I) Biotinylated | Acetylgalactosamine |
| | | (O-type, glycolipids) |
| B-1355-2 | Wisteria Floribunda Lectin (WFA, | Galactose, N- |
| | WFL) Biotinylated | Acetylgalactosamine |
| | | (O-type, glycolipids) |
| B-1245-2 | Galanthus Nivalis Lectin (GNL) | Mannose (N-type) |
| | Biotinylated | |
| B-1375-2 | Narcissus Pseudonarcissus (Daffodil) | Mannose (N-type) |
| | Lectin (NPL, NPA) biotinylated | |
| B-1005-5 | Concanavilin A (ConA) Biotinylated | Mannose, Glucose |
| B-1015-5 | Soybean Agglutinin (SBA) | N- |
| | Biotinylated | Acetylgalactosamine |
| | | (O-type, glycolipids) |
| B-1235-2 | Vicia Villosa Lectin (VVL, VVA) | N- |
| | Biotinylated | Acetylgalactosamine |
| | | (O-type, glycolipids) |
| B-1175-1 | Lycopersicon esculentum (Tomato) | N- |
| | Lectin (LEL, TL) Biotinylated | Acetylglucosamine |
| B-1165-2 | Solanum Tuberosum (Potato) Lectin | N- |
| | (STL, PL) Biotinylated | Acetylglucosamine |
| B-1025-5 | Wheat Germ Agglutinin (WGA) | N- |
| | Biotinylated | Acetylglucosamine |
| B-1215-2 | Griffonia (Bandeiraea) Simplicifolia | N- |
| | Lectin II (GSL II, BSL II) | Acetylglucosamine |
| | | (N-type) |
| B-1315-2 | Maackia Amurensis Lectin I (MAL I) | N-Acetylneuraminic |
| | Biotinylated | acid |
| B-1305-2 | Sambucus Nigra Lectin (SNA, EBL) | N-Acetylneuraminic |
| | Biotinylated | acid |

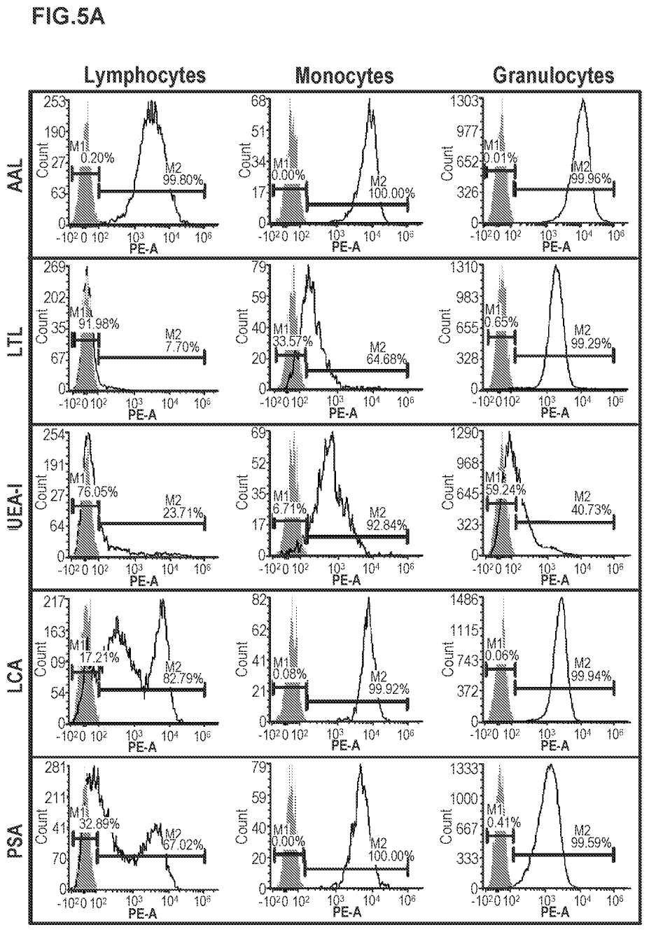

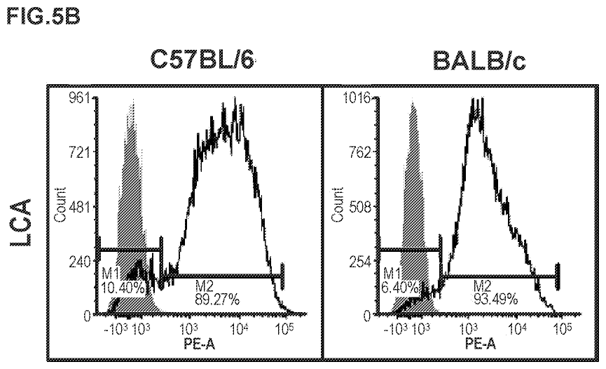

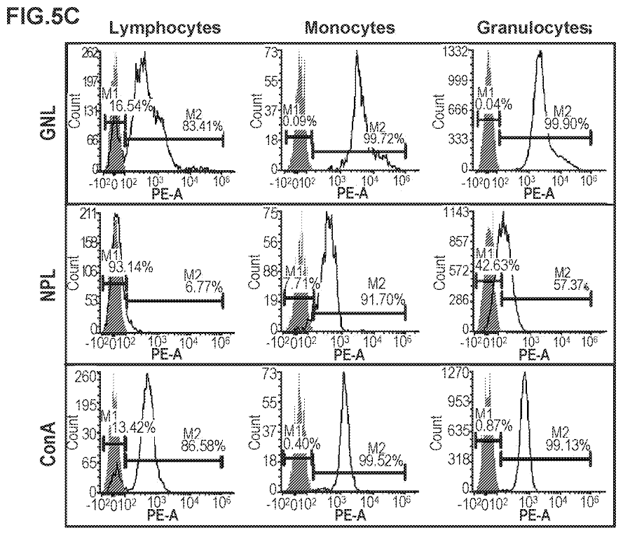

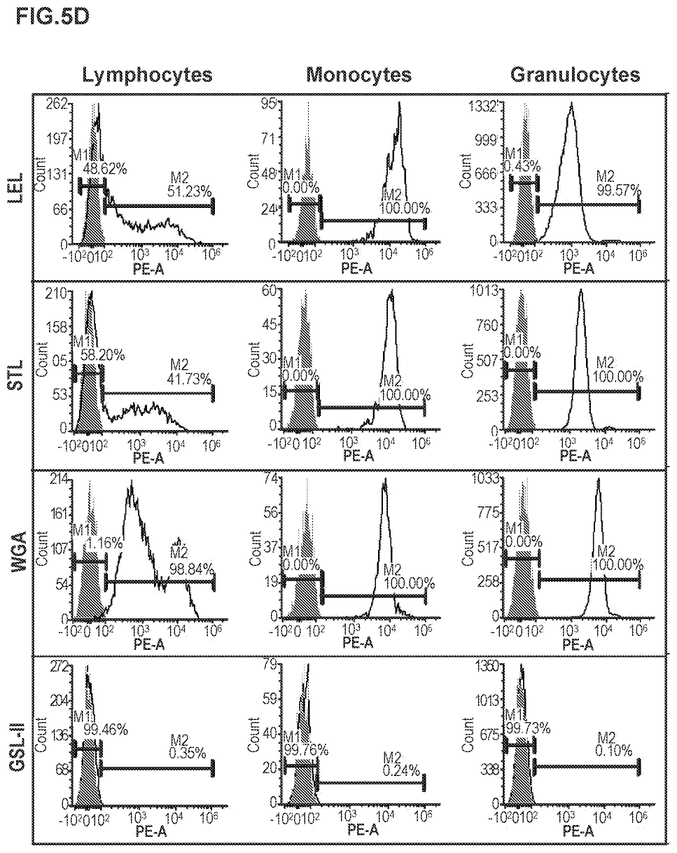

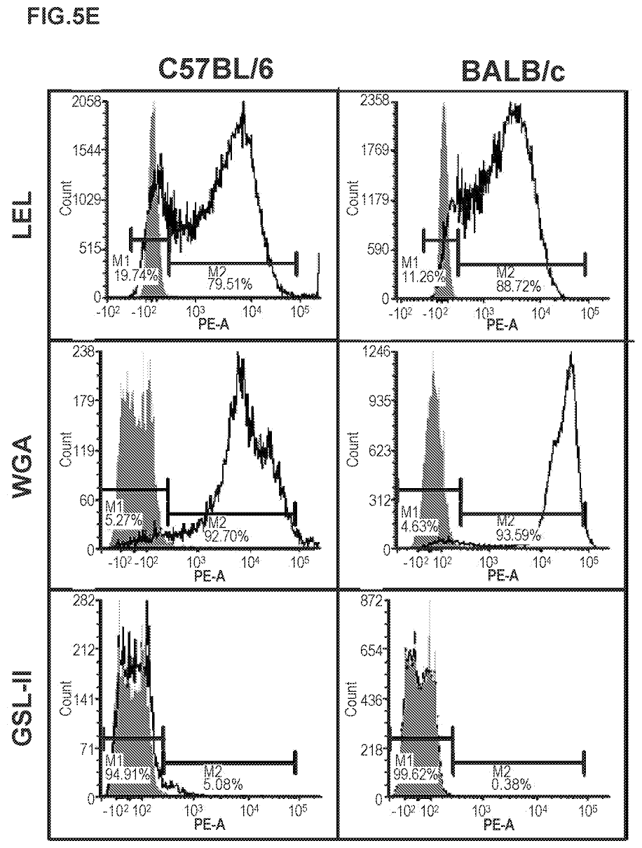

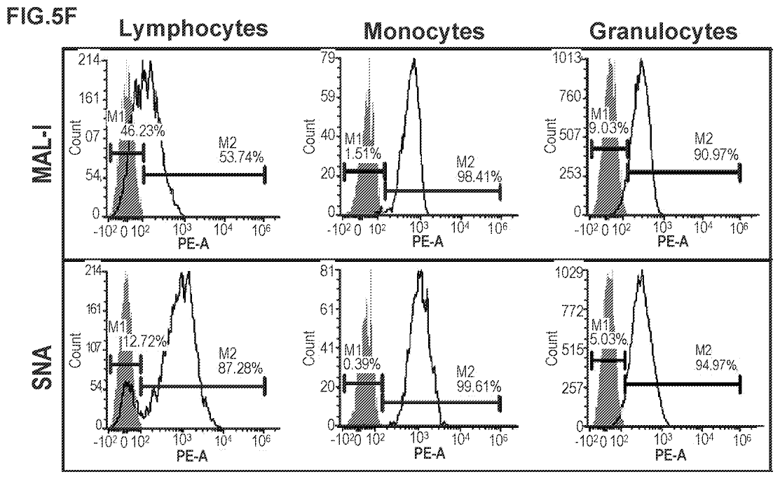

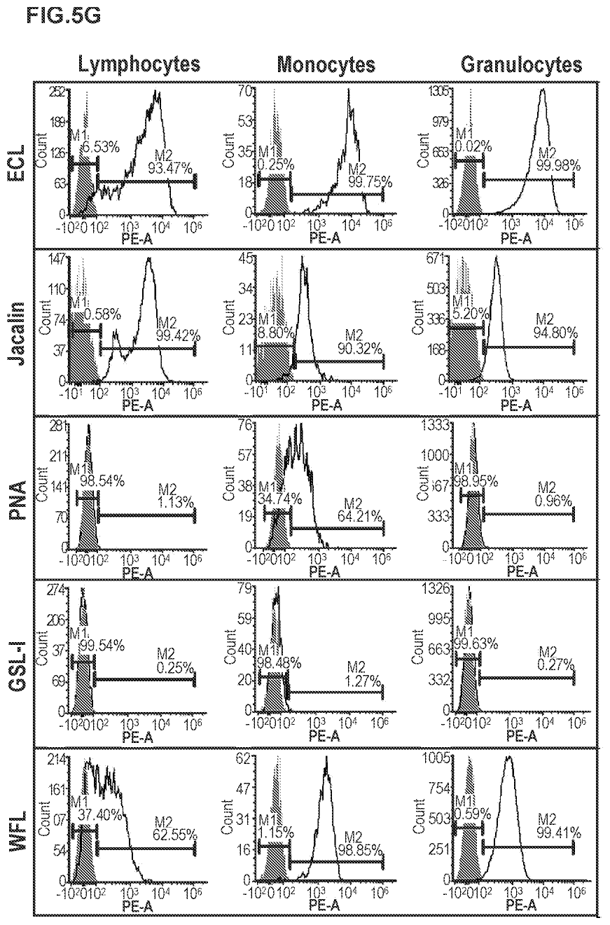

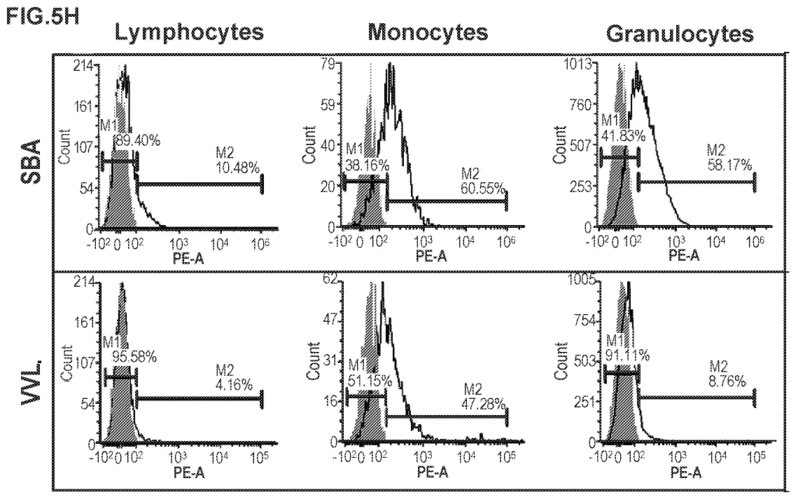

Of the exemplary tested lectins, five were classified as Fucose dependent (AAL, LTL, UEA-I, LCA, and PSA). As shown in FIG. 5A, AAL stained a large proportion of the tested cell subpopulations (lymphocytes: 99.80%; monocytes 100%; granulocytes: 99.96%). LCA and PSA identified binding motifs on all monocytes and granulocytes but did not bind to a small percentage of the lymphocytic population. However, only LCA⁻ cells have a clear negative population distribution. The identification of a discrete LCA⁻ population was also observed in murine splenic lymphoid cells from C57BL/6 and BALB/c strains (C57BL/6: 10.40%; BALB/c: 6.40%) (FIG. 5B).

Of the exemplary tested lectins, three were classified as mannose dependent (ConA, GNL and NPL). As shown in FIG. 5C, GNL and ConA had similar profiles for all subpopulations analyzed, and demonstrated staining of all monocytic (GNL⁺: 99.72%; ConA⁺: 99.52%) and granulocytic (GNL⁺: 99.90%; ConA⁺: 99.13%) populations, and showed no binding to only a small group of lymphocytes (GNL⁻: 16.54% vs ConA⁻: 13.42%). The NPL lectin identified most monocytes (91.70%), a partial majority of granulocytes (57.37%) but did not identify glyco-targets on human lymphocytes (NPL⁻: 93.14% vs NPL⁺: 6.77%).

Of the exemplary tested lectins, GSL-II, LEL, STL, WGA were considered GlcNAc dependent lectins. As shown in FIG. 5D, WGA stained all cell subpopulations (lymphocytes: 98.84%; monocytes: 100%; granulocytes 100%), and both LEL and STL, presented a specific partial staining of the lymphocytic population generating clear positive (LEL⁺: 51.23%; STL⁺: 41.73%) and negative (LEL⁻: 48.26%; STL⁻: 58.20%) populations. GSL-II demonstrated limited staining. As shown in FIG. 5E, similar staining patterns were observed in murine cell lines for lectins WGA and GSL-II, compared to humans.

Of the exemplary tested lectins, SNA and MAL-I. were classified as sialic acid-containing glycans. As shown in FIG. 5F, these lectins stained virtually all monocytes (MAL-I⁺: 98.41%; SNA⁺: 99.61%), and the vast majority of granulocytes (MAL-I⁺: 90.97%; SNA⁺: 94.97%). As for the lymphocyte population, a different glyco-profiling was observed for each lectin. Although both had larger positive populations (MAL-I⁺: 53.74%; SNA⁺: 87.28%), only the SNA⁻ cells (12.72%) could be clearly defined.

Of the exemplary tested lectins, ECL, Jacalin, PNA, GSL-I, and WFL were classified as galactose binding lectins. As shown in FIG. 5G, ECL and Jacalin demonstrated a double population for positive lymphocytes (ECL⁺: 93.47%; Jacalin⁺: 99.42%). Similarly, WFL also showed a different profiling for lymphocytes versus monocytes, and granulocytes. populations were basically absent from monocytes (1.15%) and granulocyte (0.59%) gates, but it represented close to 1/3 of lymphocytes (37.40%).

Of the exemplary tested lectins, SBA and VVL were classified as GalNAc binding lectins. As shown in FIG. 5H, SBA staining led to a larger percentage of positive cells in all three human subpopulations. The difference was marginal for lymphocytes (SBA⁺: 10.48% vs VVL⁺: 4.16%), but slightly larger for monocytes (SBA⁺: 60.55% vs VVL⁺: 47.28%), and much more accentuated for granulocytes (SBA⁺: 58.17% vs VVL⁺: 8.76%).

### Example 3. Assessment of Glycan Based Conjugation Methods

This Example describes the use of glycan conjugation methods on both lectins and antibodies to assess their effects when used for lectin panels and/or in conjunction with antibody panels. The conjugations described in this Example allow for the conjugating moiety to develop protection characteristics that prevent false positive signals due to one or more lectins being bound through the glycan on the cell surface of an antibody or additional lectin.

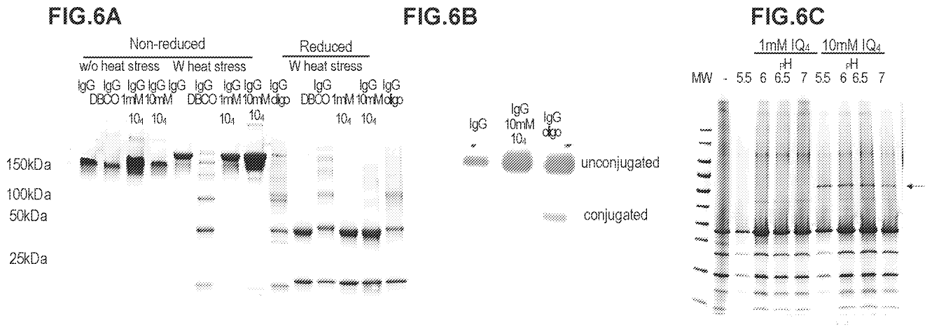

Conditions for glycan conjugation on both antibodies and lectins was assessed. Antibody stability after conjugation was assessed as follows: a mouse IgG clone (FIG. 6A lanes 1, 5, 10) was either conjugated to an oligonucleotide by DBCO hinge conjugation (FIG. 6A lanes 2, 6, 11), or periodate oxidation in two concentrations, 1 mM (FIG. 6A lanes 3, 7, 12) or 10 mM (FIG. 6A lanes 4, 8, 13) and analyzed by SDS-PAGE, using mouse IgG clone (FIG. 6A lanes 1, 5, 10) as a control. Additionally, an oligonucleotide conjugated antibody (DBCO hinge conjugation) (FIG. 6A lanes 9, 14) was used to visualize migration changes due to the presence of the oligonucleotide. Because aggregate and artifact formation are slow under recommended storage conditions (FIG. 6A lanes 1-4), accelerating conditions through heating (FIG. 6A lanes 5-9), or heating combined with reducing agent (FIG. 6A lanes 10-14), was applied. No artifact was observed without accelerating conditions. However, under heat-only stress, and heat with reducing agent stress, both samples that underwent hinge-DBCO conjugation (IgG DBCO and IgG oligo) showed artifacts formed with different sizes. Conversely, samples treated with periodate for glycan conjugation showed increased resistance to artifact formation. This result highlights that characteristics due to artifact formation after thiol-based conjugation are potentially triggered during the hinge-DBCO derivatization and are carried through the final oligo-conjugated format. This effect can be minimized, and product stability increased, by shifting to glycan-based conjugation methods.

In a similar experiment, the ability to conjugate an oligonucleotide to periodate-treated IgG was assessed (FIG. 6B). A mouse IgG clone as described above (FIG. 6B lane 1) was oxidized with 10 mM Sodium Periodate, buffer exchanged into conjugation buffer (FIG. 6B lane 2), followed by conjugation of a hydrazide-modified oligo (FIG. 6B lane 3). Each sample was loaded on an IEF gel for isoelectric separation and visualized for unconjugated and conjugated molecules. These results suggest that the IgG is modified by the oligonucleotide conjugation and further demonstrates that, even in high periodate concentration, no molecular artifact is being formed due to conjugation.

In a final experiment, the ability of conjugating a plant lectin (ConA) following the same protocol as above was assessed. Because lectins are more susceptible to pH changes than immunoglobulins, the periodate oxidation step was tested in different pHs (5.5-7). After oxidation, all samples were buffer exchanged into conjugation buffer and incubated with a hydrazide-modified oligonucleotide. As shown in FIG. 6C, for samples treated with 1 mM periodate (FIG. 6C lanes 2-5), no conjugation was observed in any of the pH tested. This result is consistent with previous findings because, unlike mammals, plant lectins do not contain sialic acid. However, when samples were oxidized under harsher condition (10 mM periodate), the final conjugate product was capable of being visualized (FIG. 6C lanes 6-9).

### Example 4. Generation and Assessment of Exemplary Lectin-Direct Conjugates as Universal Reagents for Sample Multiplexing

In view of the results indicating the feasibility of the use of lectins for the identification of a sample of origin for a cell from a pool of distinct samples as outlined in Example 1, this Example describes the generation, optimization and use of lectins directly conjugated to an oligonucleotide (lectin-direct conjugates) in single cell sequencing methods. In this Example, surface markers on the cells of each distinct sample were labeled with a unique lectin-direct conjugate prior to pooling.

### 4. A Generation of Exemplary Lectin-Direct Conjugates

Exemplary lectin-direct conjugates were prepared as follows. An exemplary lectin, Concanavalin A (Con A), was reacted with 8 equivalence of DBCO-PEG4-NHS ester in the ConA Formulation buffer (10 mM mannose, 10 mM HEPES, 0.15 M NaCl, pH 7.5, 0.01 mM MnCl2, 0.1 mM CaCl₂)) through click Chemistry Tools (Cat #A134-25) for 2 hours, and desalted with a column packed with Sephadex G-25 Medium resins (Cytiva Cat #17003302). The DBCO-modified lectin was then mixed overnight with 1 molar equivalence of an exemplary 5′-azide modified oligonucleotide, LDC1-LDC5, overnight. Exemplary oligonucleotides LDC1-LDC5 are outlined in Table E5.

TABLE E5
Exemplary oligonucleotides for generation of lectin-direct
conjugates

| Exemplary Lectin-Direct | |
|---|---|
| Conjugate Oligonucleotide | |
| Sequence | SEQ ID NO |
| LDC1 | SEQ ID NO: 14 |
| LDC2 | SEQ ID NO: 15 |
| LDC3 | SEQ ID NO: 16 |
| LDC4 | SEQ ID NO: 17 |
| LDC5 | SEQ ID NO: 18 |

Three conditions, SEC only, AEX only, AEX and SEC, were tested for the purification of the resulting exemplary lectin-direct conjugates to remove unconjugated oligonucleotides and ConA, as follows.

For SEC only purification, lectin-direct conjugates were loaded onto Superdex-200 resin and eluted with ConA Formulation Buffer (10 mM mannose, 10 mM HEPES, 0.15 M NaCl, 0.01 mM MnCl₂, 0.1 mM CaCl₂, pH 7.5). Fractions were combined, concentrated in a centrifugal filter (MWCO 50 kDa, Millipore, Cat #UFC9050), and underwent buffer exchange into the ConA Final Buffer (10 mM HEPES, 0.15 NaCl, pH 7.5, 0.01 mM MnCl₂, 0.1 mM CaCl₂, 0.08% NaN₃). The pooled solution was concentrated by centrifugal filters and diluted to the desirable concentration for the subsequent testing.

For AEX only purification, lectin-direct conjugates were loaded on a column with Macro-Prep DEAE Resin (BioRad Cat #1560020), washed with 2 column volumes of 0.5 M NaCl, 10 mM HEPES, 0.15 M NaCl, 0.01 mM MnCl₂, 0.1 mM CaCl₂, pH 7.5, and eluted with 4 column volumes of 1.0 M NaCl, 10 mM HEPES, 0.15 M NaCl, pH 7.5, 0.01 mM MnCl₂, 0.1 mM CaCl₂. Eluted solutions were combined, washed with 2 M Tris, pH 8.0, for 5 times and concentrated in a centrifugal filter (MWCO 50 kDa, Millipore, Cat #UFC9050). The eluted fractions are combined and underwent buffer exchange into the ConA Final Buffer (10 mM HEPES, 0.15 NaCl, pH 7.5, 0.01 mM MnCl₂, 0.1 mM CaCl₂, 0.08% NaN₃). The pooled solution was concentrated by centrifugal filters and diluted to the desirable concentration for the subsequent testing.

For SEC and AEX purification, lectin-direct conjugates were loaded on a column with Macro-Prep DEAE Resin (BioRad Cat #1560020), washed with 2 column volumes of 0.5 M NaCl, 10 mM HEPES, 0.15 M NaCl, 0.01 mM MnCl₂, 0.1 mM CaCl₂, pH 7.5, and eluted with 4 column volumes of 1.0 M NaCl, 10 mM HEPES, 0.15 M NaCl, pH 7.5, 0.01 mM MnCl₂, 0.1 mM CaCl₂. Eluted solutions were combined, washed with 2 M Tris, pH 8.0, for 5 times and concentrated in a centrifugal filter (MWCO 50 kDa, Millipore, Cat #UFC9050). The eluted fractions are combined and underwent buffer exchange into the ConA Final Buffer (10 mM HEPES, 0.15 NaCl, pH 7.5, 0.01 mM MnCl₂, 0.1 mM CaCl₂, 0.08% NaN₃).

The solution was subsequently loaded on a Superdex-200 column, washed with ConA Final Buffer (10 mM HEPES, 0.15 NaCl, pH 7.5, 0.01 mM MnCl₂, 0.1 mM CaCl₂, 0.08% NaN₃), and then eluted with ConA Formulation Buffer (10 mM mannose, 10 mM HEPES, 0.15 M NaCl, pH 7.5, 0.01 mM MnCl₂, 0.1 mM CaCl₂). The eluted fractions after SEC were combined and underwent buffer exchange into the ConA Final Buffer. The pooled solution was concentrated by centrifugal filters and diluted to the desirable concentration for the subsequent testing.

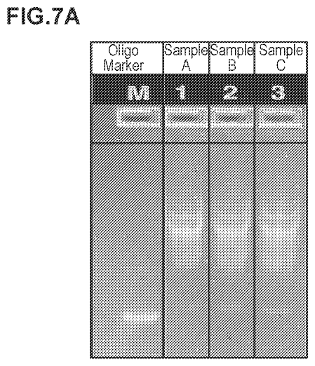

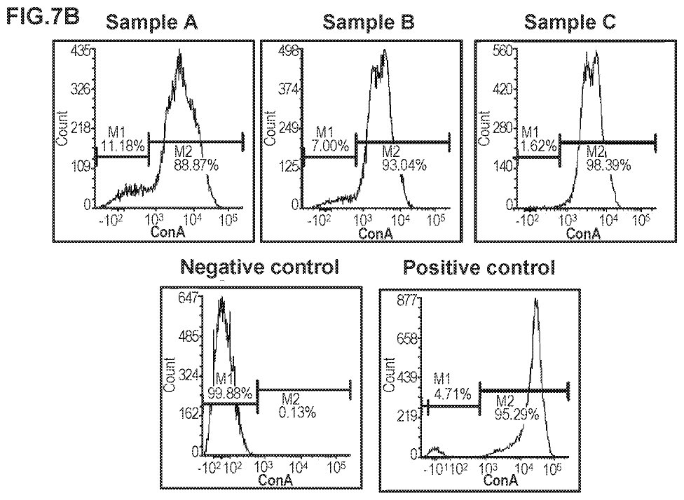

Samples from SEC only (Sample A), AEX only (Sample B) and SEC and AEX purification (Sample C) were stained with SYBR gold nucleic acid stain (ThermoFisher), loaded onto pre-cast 4% agarose gels, and imaged for DNA staining. As shown in FIG. 7A, all three purification methods resulted in similar sample recovery.

To detect the purification protocol that would yield the lowest background and maximum signal, all samples were further analyzed by Flow Cytometry. Briefly, 2 μg of exemplary lectin-direct conjugates were hybridized with a fluorescent label (oligo-dT-AF647; IDT) at room temperature for 15 minutes and used to stain human PBMCs. Mock sample (oligo-dT-AF647 in PBS) was used as negative control and Biotinylated-ConA (SAV-AF647; BioLegend Cat. No. 405237) was used as positive control. As shown in FIG. 7B, all three purifications resulted in equivalent staining intensity. However, the combination of AEX and SEC purification methods (Sample C) resulted in almost complete elimination of background staining (MT:1.62% for Sample C, versus M1:7.00% for Sample B and 11.18% for Sample A). These results demonstrate the ability of the exemplary conjugation and purification methods to efficiently label lectins as direct-conjugates, and that the combination of AEX and SEC purification methods results in optimized conjugation by highest removal of unconjugated oligonucleotides and lectins.

### 4.B Assessment of Exemplary Lectin-Direct Conjugate Labeling of Immune Cells.

Fresh human PBMCs from a single donor were isolated as described in Example 1C above. Five aliquots of the same tube of fresh human PBMCs were labeled with 0.25 μg of an exemplary protein identifying antibody based HTO, and 0.1 μg of unique exemplary lectin-direct conjugates prepared in Example 4.A above. Cells were pooled and loaded on one 10× chromium channel, and library preparation, demultiplexing and results evaluated as described in Example 1 above.

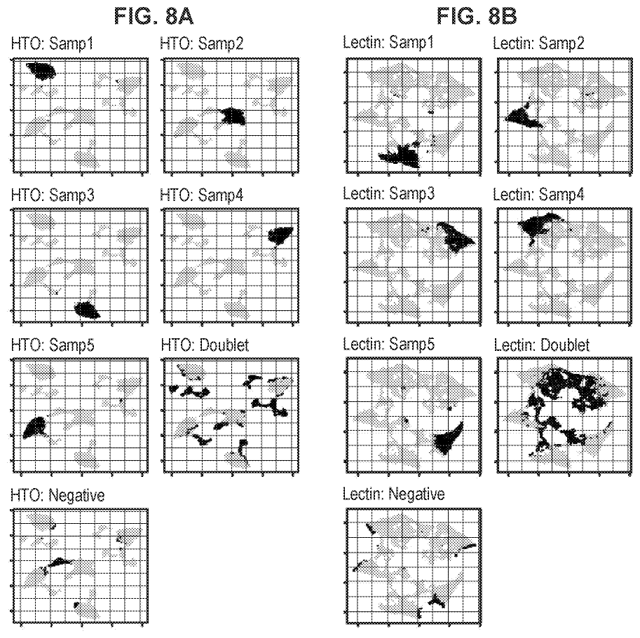

FIGS. 8A and 8B show uniform manifold approximation and projection (UMAP) plots of individual cell populations from each aliquot of PBMC's.

These results demonstrate the ability of exemplary lectin-direct conjugates were capable of labeling and detecting specific cell populations using less than half the concentration of antibody based HTOs.

### Example 5. Assessment of Multiple Exemplary Lectin-Direct Conjugates as Reagents for Sample Identification and Characterization

This Example describes the generation, optimization and use of distinct lectins directly conjugated to an oligonucleotide (lectin-direct conjugates) in single cell sequencing methods.

### 5.A Generation of Multiple Exemplary Lectin-Direct Conjugates

Exemplary lectin-direct conjugates are prepared as follows. Exemplary lectins Concanavalin A (Con A), wheat germ agglutinin (WGA), Phaseolus vulgaris (red kidney bean) erythroagglutinin, a Lens culinaris (lentil) agglutinin, Datura stramonium agglutinin, Sambucus nigra (elderberry bark) agglutinin, Snowdrop lectin (GNA), Peanut agglutinin (PNA), Jacalin (AIL), Hairy vetch lectin (VVL), Maackia amurensis leukoagglutinin (MAL), and Maackia amurensis hemoagglutinin are reacted with 8 equivalence of DBCO-PEG4-NHS ester in a Formulation buffer (10 mM mannose, 10 mM HEPES, 0.15 M NaCl, pH 7.5, 0.01 mM MnCl2, 0.1 mM CaCl₂)) through click Chemistry Tools (Cat #A134-25) for 2 hours, and are desalted with a column packed with Sephadex G-25 Medium resins (Cytiva Cat #17003302). The DBCO-modified lectins are then mixed overnight with 1 molar equivalence of an exemplary 5′-azide modified oligonucleotide overnight.

The resulting exemplary lectin-direct conjugates are then purified to remove unconjugated oligonucleotides and unconjugated lectins.

### 5.B Assessment of Exemplary Lectin-Direct Conjugate Labeling of Immune Cells.

Fresh human PBMCs from a single donor are isolated as described in Example IC above. Five aliquots of the same tube of fresh human PBMCs are labeled with unique HTOs and exemplary lectin-direct conjugates prepared in Example 5.A above. Cells are pooled and loaded on one 10× chromium channel, and library preparation, demultiplexing and results are evaluated as described in Example 1 above.

### 5.C Assessment of Exemplary Lectin-Direct Conjugate Labeling of Sub-Cellular Components.

Fresh human PBMCs from a single donor are isolated as described in Example IC above, followed by isolation of nuclei. Five aliquots of the same tube of freshly isolated nuclei are labeled with unique HTOs and exemplary lectin-direct conjugates prepared in Example 5.A above. Isolated nuclei are pooled and loaded on one 10× chromium channel, and library preparation, demultiplexing and results are evaluated as described in Example 1 above.

### Example 6. Assessment of Exemplary Lectins as Reagents for Spatial Applications and Tissue Characterization

This Example describes the use of distinct lectins for identification of tissue structures and alterations in tissue architecture applicable to the field of spatial transcriptomics.

Briefly, FFPE blocks were prepared containing biopsy samples of melanoma, colorectal adenocarcinoma or normal adjacent tissue (NAT) from two separate donors D1 and D2, respectively, for each sample type. FFPE samples were then blocked with 50-100 μL of Blocking solution [1×PBS, 0.05% Tween20, Sheared Salmon Sperm DNA, and 10% Fetal Bovine Serum] for a minimum of 15 minutes prior to staining. FFPE samples were stained with a primary antibody solution by adding 0.5 μg of the primary antibody of interest (Melan-A, EpCAM or Sox10) to 100 μL of Blocking solution, and co-stained with various lectins (Jacalin, LEL, TL, SNL, EBL or UEA-I) by adding 0.05 μg of the selected biotinylated Lectin from Vector Labs, for 1.5 hours. FFPE samples were then washed with blocking solution and incubated with 50-100 μL of secondary antibody staining solution containing 0.25 μg of the secondary antibody of interest, 0.25 μg of Goat anti-Biotin secondary Ab, conjugated to DyLight 649 from Rockland Labs, and 5 ng DAPI for 1.5 hours. FFPE samples were washed 4× using 75 μL Wash buffer [1×PBS with 0.05% Tween], and imaged after a minimum of 20 minutes of drying.

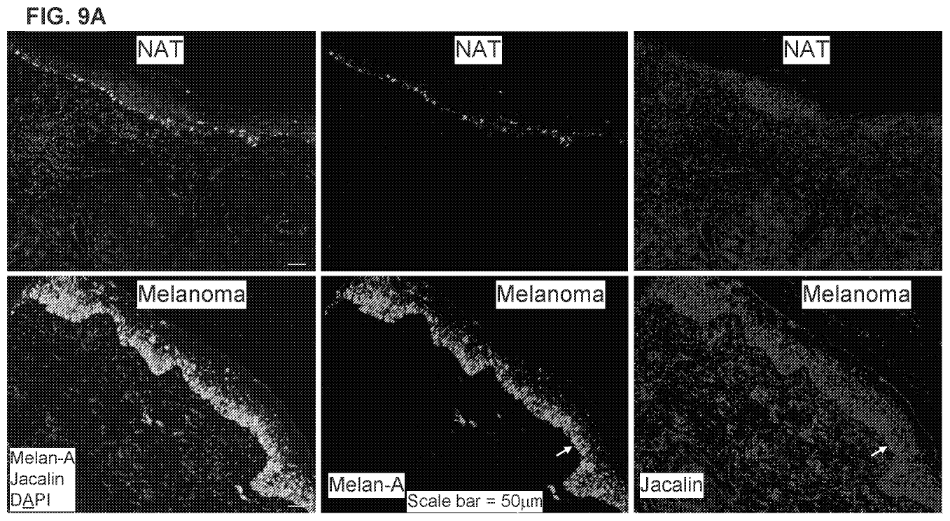

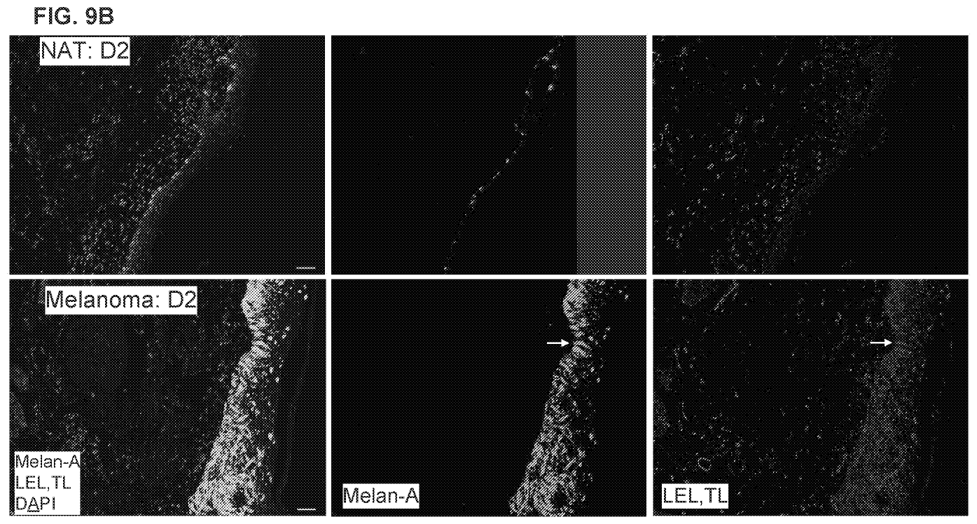

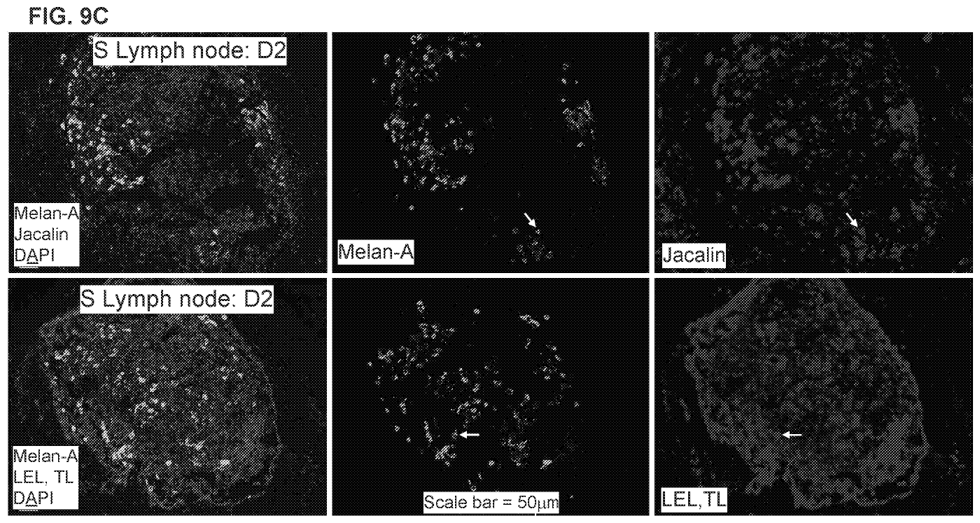

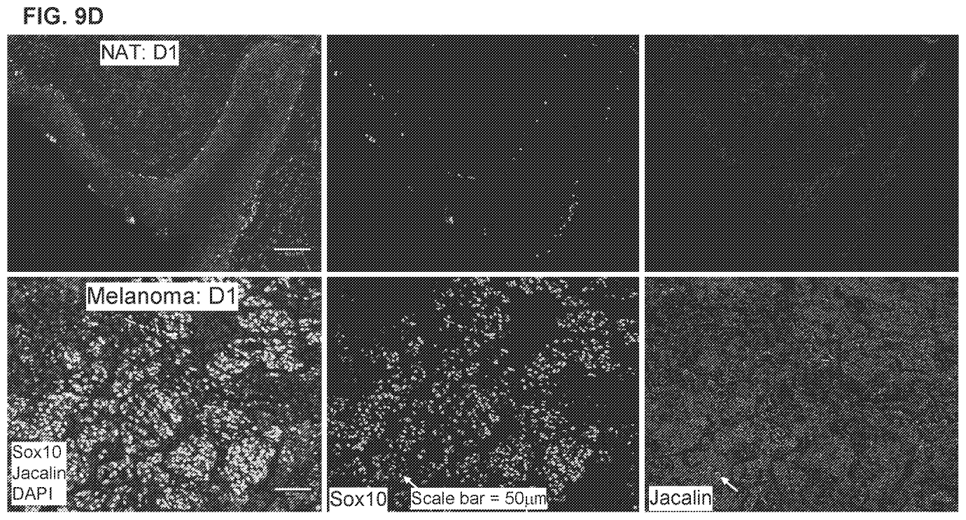

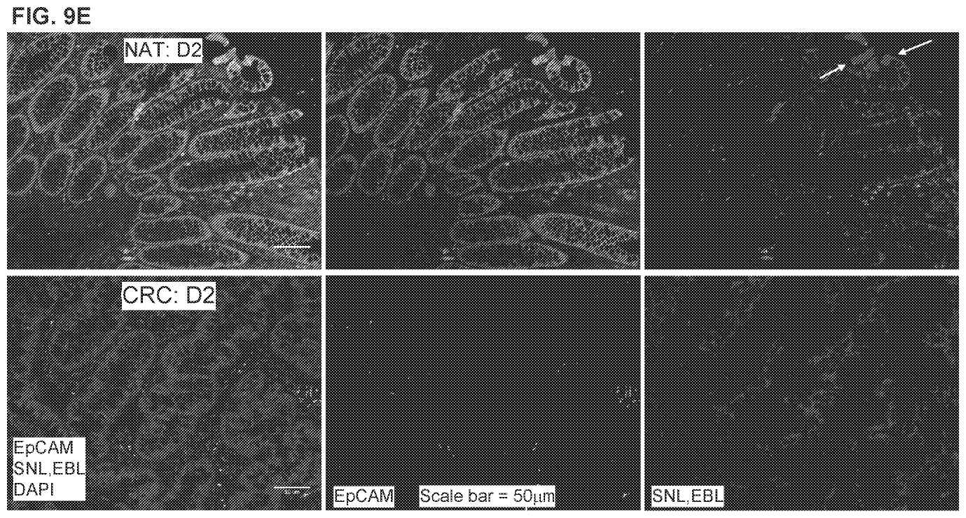

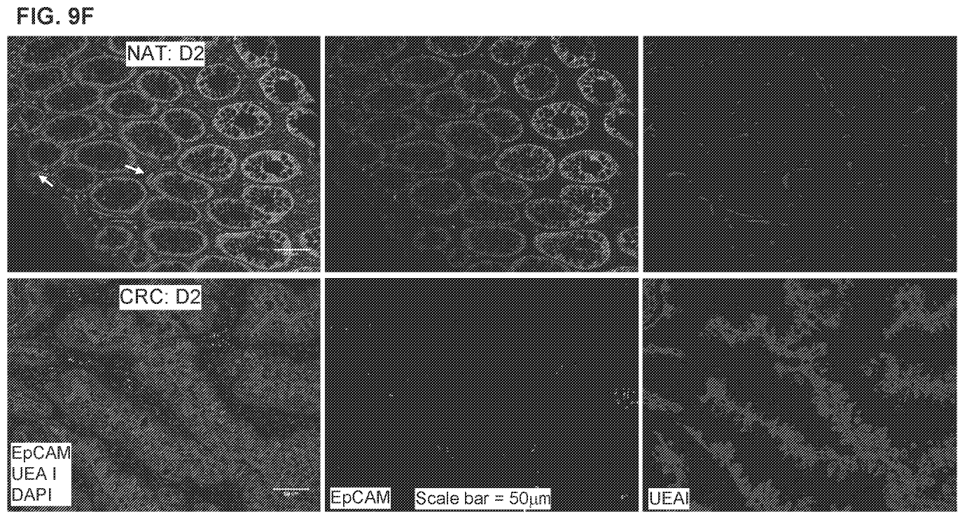

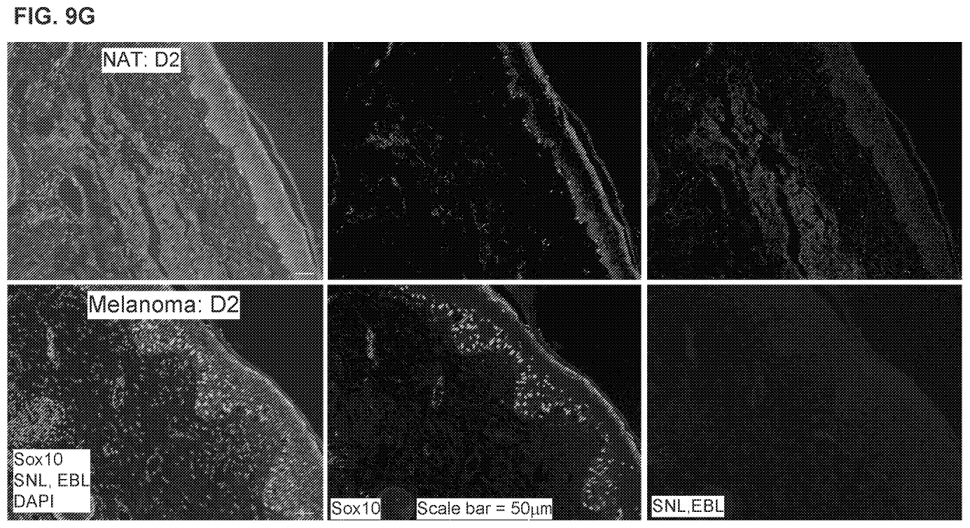

As shown in FIG. 9A, cytoplasmic Melan-A expression is confined to melanocytes in stratum basale (top left panel), whereas malignant cells retaining Melan-A expression penetrate deeper into the dermal layer (bottom left panel; top and bottom middle panels show single overlays in green). Jacalin lectin binding is present in keratinocytes and to a lesser extent in the dermal layer in NAT (top left panel). Significant Jacalin lectin binding is additionally detected in melanoma cells and scattered cells in the dermal layer (bottom left panel; see top bottom right panels for single overlays). Split channels reveal co-localization of Melan-A and Jacalin Lectin staining on Melanoma cells noted in the tumor (see middle, right bottom single overlays; arrows).

As shown in FIG. 9B, cytoplasmic Melan-A expression is confined to melanocytes in stratum basale (top left panel), whereas malignant cells retaining Melan-A expression penetrate deeper into the dermal layer (bottom left panel; top and bottom middle panels show single overlays in green). Strong LEL and TL lectin binding is present on leukocytes present in the dermal layer in both NAT and cancer tissues (top and bottom left panels). Notable LEL and TL lectin binding is additionally detected specifically in melanoma cells (bottom left panel; see top bottom right panels for single overlays). Split channels reveal co-localization of Melan-A and LEL and TL Lectin staining on Melanoma cells noted in the tumor (see middle, bottom single overlays; arrows).

As shown in FIG. 9C, Jacalin binding is detected specifically in melanoma cells that have infiltrated nearby sentinel (S) lymph nodes (top left panel), which is also true for LEL, TL binding, with a distribution across multiple cells/structures (bottom left panel). Split channels reveal co-localization of Melan-A and Lectin staining on Melanoma cells noted in the cancer field of view (see top and bottom, middle and right, single overlays; arrows).

As shown in FIG. 9D, nuclear Sox 10 expression is confined to melanocytes in stratum basale (top left panel), whereas malignant cells retaining Sox10 expression penetrate deeply into the dermal layer (bottom left panel; see top and bottom middle panels for single overlays. Jacalin lectin binding is weakly present in keratinocytes and to a greater extent in sebocytes from oil glands in NAT (top left panel). Jacalin lectin binding is additionally intimately associated with malignant cells and stromal cells detected inside of the tumor (bottom left panel; see top and bottom right panels for single overlays). Split channels reveal co-localization of Sox10 and Jacalin lectin staining on Melanoma cells noted in the cancer field of view (see middle, right bottom single overlays; arrows).

As shown in FIG. 9E, EpCAM is confined to a single layer of glandular cells composing normal crypt architecture (top left panel), whereas malignant cells forming multi-layer irregularly branched crypts do not have EpCAM expression (bottom left panel; see top and bottom middle panels for single overlays). SNL and EBL lectin binding is mostly only detected in intra-crypt spaces present in tumor tissue (bottom left panel; see top and bottom right panels for single overlays), and can occasionally be detected in normal colon (see top right panel; arrows).

As shown in FIG. 9F, EpCAM is confined to a single layer of glandular cells composing normal crypt architecture (top left panel), whereas malignant cells forming multi-layer irregularly branched crypts do not have EpCAM expression (bottom left panel; see top and bottom middle panels for single overlays). UEA-I lectin binding is strongest in endothelial cells (top left panel; see arrows) and is also detected in intra-crypt spaces present in tumor tissue (bottom left panel), see top and bottom right panels for single overlays).

As shown in FIG. 9G, nuclear Sox 10 expression is confined to melanocytes in stratum basale from skin (top left panel), whereas malignant cells retaining Sox10 expression penetrate deeper into the dermal layer (bottom left panel; top and bottom middle panels show single overlays). SNL and EBL lectin binding is notably absent in keratinocytes (top left panel), and likewise is completely absent from melanoma cells (bottom left panel; see top and bottom right panels for single overlays).

These results demonstrate the ability to detect tissue alterations due to underlying disease states using lectins as staining reagents.

SEQUENCES

| SEQ ID | | |
|---|---|---|
| NO | SEQUENCE | DESCRIPTION |
| 1 | GTGACTGGAGTTCAGACGTGTGCTCTTCCGATCT | ABT1 with poly-A |
| | GGTAGTCATCGTGGTAAAAAAAAAAAAAAAAAA | tail |
| | AAAAAAAAAA*A*A | |
| 2 | GTGACTGGAGTTCAGACGTGTGCTCTTCCGATCT | ABT2 with poly-A |
| | GTCCGTATTCTGTCAAAAAAAAAAAAAAAAAAA | tail |
| | AAAAAAAAAA*A*A | |
| 3 | GTGACTGGAGTTCAGACGTGTGCTCTTCCGATCT | ABT3 with poly-A |
| | TCCCTTTATGCGAGGAAAAAAAAAAAAAAAAAA | tail |
| | AAAAAAAAAA*A*A | |
| 4 | GTGACTGGAGTTCAGACGTGTGCTCTTCCGATCT | ABT4 with poly-A |
| | AAAGCATGACCAGTAAAAAAAAAAAAAAAAAAA | tail |
| | AAAAAAAAAA*A*A | |
| 5 | GTGACTGGAGTTCAGACGTGTGCTCTTCCGATCT | ABT5 with poly-A |
| | TACAGTCGCTTGGCAAAAAAAAAAAAAAAAAAA | tail |
| | AAAAAAAAAA*A*A | |
| 6 | GTCAACTCTTTAGCG | HTO A0251 |
| | | barcode sequence |
| | | for |
| | | Na+/K+ ATPase |
| | | β3 |
| 7 | TGATGGCCTATTGGG | HTO A0252 |
| | | barcode sequence |
| | | for Na+/K+ |
| | | ATPase β3 |
| 8 | TTCCGCCTCTCTTTG | HTO A0253 |
| | | barcode sequence |
| | | for Na+/K+ |
| | | ATPase β3 |
| 9 | ACCCACCAGTAAGAC | HTO A0301 |
| | | barcode sequence |
| | | for mouse major |
| | | histocompatibility |
| | | complex H-2 |
| 10 | GGTCGAGAGCATTCA | HTO A0302 |
| | | barcode sequence |
| | | for mouse major |
| | | histocompatibility |
| | | complex H-2 |
| 11 | AAAGCATTCTTCACG | HTO A0304 |
| | | barcode sequence |
| | | for Mouse major |
| | | histocompatibility |
| | | complex H-2, |
| | | MHC, T200, Ly-5, |
| | | LCA |
| 12 | CTTGCCGCATGTCAT | HTO A0303 |
| | | barcode sequence |
| | | for |
| | | Mouse major |
| | | histocompatibility |
| | | complex H-2, |
| | | MHC, T200, Ly-5, |
| | | LCA |
| 13 | CTTTGTCTTTGTGAG | HTO A0305 for |
| | | barcode sequence |
| | | for Mouse major |
| | | histocompatibility |
| | | complex H-2, |
| | | MHC, T200, Ly-5, |
| | | LCA |
| 14 | GTGACTGGAGTTCAGACGTGTGCTCTTCCGATCT | LDC1 with poly-A |
| | ACCCACTTCAGTTCGAAAAAAAAAAAAAAAAAA | tail |
| | AAAAAAAAAA*A*A | |
| 15 | GTGACTGGAGTTCAGACGTGTGCTCTTCCGATCT | LDC2 with poly-A |
| | ACGCCGGTAGTATCTAAAAAAAAAAAAAAAAAA | tail |
| | AAAAAAAAAA*A*A | |
| 16 | GTGACTGGAGTTCAGACGTGTGCTCTTCCGATCT | LDC3 with poly-A |
| | TATCGTCTCCGACTAAAAAAAAAAAAAAAAAAA | tail |
| | AAAAAAAAAA*A*A | |
| 17 | GTGACTGGAGTTCAGACGTGTGCTCTTCCGATCT | LDC4 with poly-A |
| | AGGTCCTCCTGTATTAAAAAAAAAAAAAAAAAA | tail |
| | AAAAAAAAAA*A*A | |
| 18 | GTGACTGGAGTTCAGACGTGTGCTCTTCCGATCT | LDC5 with poly-A |
| | ATTTCGCCTTGCCAGAAAAAAAAAAAAAAAAAA | tail |
| | AAAAAAAAAA*A*A | |

1-38. (canceled)

39. A method comprising:
(a) contacting a plurality of target cells comprising one or more glycans with a first plurality of complexes, wherein:
the first plurality of complexes comprise a first glycan-binding agent and a first labeling oligonucleotide,
the first glycan-binding agent binds to all or a portion of a first glycan on the one or more target cells, and
the first glycan-binding agent is a first lectin;
(b) detecting the first labeling oligonucleotide; and
(c) identifying a first glycan on the one or more target cells according to the detection of the first labeling oligonucleotide.

40. The method of claim 39, wherein first glycan-binding agent is linked to the first labeling oligonucleotide.

41. The method of claim 39, wherein the first plurality of complexes further comprise biotin linked to the first glycan-binding agent and the first labeling oligonucleotide.

42. The method of claim 39, wherein the first plurality of complexes further comprise (i) biotin linked to the first glycan-binding agent, and (ii) an anti-biotin antibody bound to the biotin and linked to the first labeling oligonucleotide.

43. The method of claim 39, wherein the first plurality of complexes further comprise (i) biotin linked to the first glycan-binding agent, and (ii) streptavidin bound to the biotin and linked to the first labeling oligonucleotide.

44. The method of claim 39, wherein first labeling oligonucleotide further comprises one or more of:
one or more detectable labels,
first barcode, wherein the first barcode is specific for the first glycan,
a binding site for a primer, and
a capture sequence.

45. The method of claim 44, wherein the one or more detectable labels comprise one or more of radioisotopes, fluorophores, chemiluminescent compounds, bioluminescent compounds, and dyes.

46. The method of claim 44, wherein the detecting in (b) comprises qualitative or quantitative detection of the one or more detectable labels.

47. The method of claim 39, further comprising after (a) partitioning the plurality of target cells into a plurality of partitions, wherein each partition of the plurality of partitions comprises a single target cell associated with a first complex.

48. The method of claim 39,
wherein (a) further comprises contacting the plurality of target cells with one or more additional pluralities of complexes, wherein:
the one or more additional plurality of complexes comprise one or more additional glycan-binding agents and one or more additional labeling oligonucleotides,
the one or more additional glycan-binding agents bind to all or a portion of one or more additional glycans on the one or more target cells, and
the one or more additional glycan-binding agents are one or more additional lectins;
wherein (b) further comprises detecting the one or more additional labeling oligonucleotides; and
wherein (c) further comprises identifying one or more additional glycans on the one or more target cells according to detection of the one or more additional labeling oligonucleotides.

49. The method of claim 48, wherein the one or more additional glycan-binding agents are linked to the one or more additional labeling oligonucleotides.

50. The method of claim 48, wherein the one or more additional pluralities of complexes further comprise biotin linked to the one or more additional glycan-binding agents and the one or more additional labeling oligonucleotides.

51. The method of claim 48, wherein the one or more additional pluralities of complexes further comprise (i) biotin linked to the one or more additional glycan-binding agents, and (ii) an anti-biotin antibody bound to the biotin and linked to the one or more additional labeling oligonucleotides.

52. The method of claim 48, wherein the one or more additional pluralities of complexes further comprise (i) biotin linked to the one or more additional glycan-binding agents, and (ii) streptavidin bound to the biotin and linked to the one or more additional labeling oligonucleotides.

53. The method of claim 48, wherein the one or more additional labeling oligonucleotides further comprise one or more of:
one or more detectable labels,
one or more additional barcodes, wherein the one or more additional barcodes are specific for the one or more additional glycans,
a binding site for a primer, and
a capture sequence.

54. The method of claim 53, wherein the one or more detectable labels comprise one or more of radioisotopes, fluorophores, chemiluminescent compounds, bioluminescent compounds, and dyes.

55. The method of claim 53, wherein the detecting in (b) comprises qualitative or quantitative detection of the one or more detectable labels.

56. The method of claim 48, further comprising after (a) partitioning the plurality of target cells into a plurality of partitions, wherein each partition of the plurality of partitions comprises a single target cell associated with the first complex or one of the additional complexes.

57. The method of claim 39, wherein the detecting in (b) comprises sequencing.

58. The method of claim 39, wherein the detecting in (b) comprises single cell sequencing.
